SEQUENCE LISTING (48 sequences)

SEQ ID NO: 1 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLSGEVFGGGTKLTVL

SEQ ID NO: 2 - protein, 118 aa, Artificial Sequence
  Synthetic sequence
EVQLLESGGGLVQPGGSLRLSCAASGFTFSYYYMQWVRQAPGKGLEWVSYIGSSGGVTNYADSVKGRFTISRDNSKNTLYLQMNSLRAEDTAVYYCARVGLGDAFDIWGQGTMVTVSS

SEQ ID NO: 3 - protein, 110 aa, Artificial Sequence
  Synthetic sequence
QSVLTQPPSASGTPGQRVTISCSGSLSNIGLNYVSWYQQLPGTAPKLLISRNNQRPSGVPDRFSGSKSGTSASLAISGLRSEDEADYYCAAWDDSPPGEAFGGGTKLTVL

SEQ ID NO: 4 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDGLPGEVFGGGTKLTVL

SEQ ID NO: 5 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLIGEVFGGGTKLTVL

SEQ ID NO: 6 - protein, 110 aa, Artificial Sequence
  Synthetic sequence
QSVLTQPPSASGTPGQRVTISCRGSSSNIGLNYVSWYQQLPGTAPKLLISRNNQRPSGVPDRFSGSKSGTSASLAISGLRSEDEADYYCAAWDDSLSGEVFGGGTKLTVL

SEQ ID NO: 7 - protein, 110 aa, Artificial Sequence
  Synthetic sequence
QSVLTQPPSASGTPGQRVTISCSGSLSNIGLNYVSWYQQLPGTAPKLLISRNNQRPSGVPDRFSGSKSGTSASLAISGLRSEDEADYYCAAWDDSLSGEVFGGGTKLTVL

SEQ ID NO: 8 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLPGEVFGGGTKLTVL

SEQ ID NO: 9 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLSGEAFGGGTKLTVL

SEQ ID NO: 10 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSPSGEVFGGGTKLTVL

SEQ ID NO: 11 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSLRGEVFGGGTKLTVL

SEQ ID NO: 12 - protein, 118 aa, Artificial Sequence
  Synthetic sequence
EVQLLESGGGLVQPGGSLRLSCAASGFTFSYYYMQWVRQAPGKGLEWVSIIGSSGGVTNYADSVKGRFTISRDNSKNTLYLQMNSLRAEDTAVYYCARVGLGDAFDIWGQGTMVTVSS

SEQ ID NO: 13 - protein, 118 aa, Artificial Sequence
  Synthetic sequence
EVQLLESGGGLVQPGGSLRLSCAASGFTFSYYYMQWVRQAPGKGLEWVSVIGSSGGVTNYADSVKGRFTISRDNSKNTLYLQMNSLRAEDTAVYYCARVGLGDAFDIWGQGTMVTVSS

SEQ ID NO: 14 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSPPGEAFGGGTKLTVL

SEQ ID NO: 15 - protein, 110 aa, Artificial Sequence
  Synthetic sequence
QSVLTQPPSASGTPGQRVTISCRGSSSNIGLNYVSWYQQLPGTAPKLLISRNNQRPSGVPDRFSGSKSGTSASLAISGLRSEDEADYYCAAWDDSPPGEAFGGGTKLTVL

SEQ ID NO: 16 - protein, 110 aa, Artificial Sequence
  Synthetic sequence
QSVLTQPPSASGTPGQRVTISCSGSSSNIGLNYVSWYQQLPGTAPKLLISRNNQRPSGVPDRFSGSKSGTSASLAISGLRSEDEADYYCAAWDDSPSGEAFGGGTKLTVL

SEQ ID NO: 17 - protein, 110 aa, Artificial Sequence
  Synthetic sequence
QYELTQPPSASGTPGQRVTMSCSGSSSNIGLNYVSWYQQLPGTAPKLLISRNNQRPSGVPDRFSGSKSGTSASLAISGLRSEDEADYYCAAWDDSLSGEVFGGGTKLTVL

SEQ ID NO: 18 - protein, 13 aa, Artificial Sequence
  Synthetic sequence
SGSSSNIGLNYVS

SEQ ID NO: 19 - protein, 13 aa, Artificial Sequence
  Synthetic sequence
SGSLSNIGLNYVS

SEQ ID NO: 20 - protein, 13 aa, Artificial Sequence
  Synthetic sequence
RGSSSNIGLNYVS

SEQ ID NO: 21 - protein, 7 aa, Artificial Sequence
  Synthetic sequence
RNNQRPS

SEQ ID NO: 22 - protein, 11 aa, Artificial Sequence
  Synthetic sequence
AAWDDSLSGEV

SEQ ID NO: 23 - protein, 11 aa, Artificial Sequence
  Synthetic sequence
AAWDDSPPGEA

SEQ ID NO: 24 - protein, 11 aa, Artificial Sequence
  Synthetic sequence
AAWDDSPSGEA

SEQ ID NO: 25 - protein, 11 aa, Artificial Sequence
  Synthetic sequence
AAWDDSLPGEV

SEQ ID NO: 26 - protein, 11 aa, Artificial Sequence
  Synthetic sequence
AAWDDGLPGEV

SEQ ID NO: 27 - protein, 11 aa, Artificial Sequence
  Synthetic sequence
AAWDDSLIGEV

SEQ ID NO: 28 - protein, 11 aa, Artificial Sequence
  Synthetic sequence
AAWDDSLSGEA

SEQ ID NO: 29 - protein, 11 aa, Artificial Sequence
  Synthetic sequence
AAWDDSPSGEV

SEQ ID NO: 30 - protein, 11 aa, Artificial Sequence
  Synthetic sequence
AAWDDSLRGEV

SEQ ID NO: 31 - protein, 5 aa, Artificial Sequence
  Synthetic sequence
YYYMQ

SEQ ID NO: 32 - protein, 17 aa, Artificial Sequence
  Synthetic sequence
YIGSSGGVTNYADSVKG

SEQ ID NO: 33 - protein, 17 aa, Artificial Sequence
  Synthetic sequence
IIGSSGGVTNYADSVKG

SEQ ID NO: 34 - protein, 17 aa, Artificial Sequence
  Synthetic sequence
VIGSSGGVTNYADSVKG

SEQ ID NO: 35 - protein, 9 aa, Artificial Sequence
  Synthetic sequence
VGLGDAFDI

SEQ ID NO: 36 - protein, 30 aa, Artificial Sequence
  Synthetic sequence
EVQLLESGGGLVQPGGSLRLSCAASGFTFS

SEQ ID NO: 37 - protein, 14 aa, Artificial Sequence
  Synthetic sequence
WVRQAPGKGLEWVS

SEQ ID NO: 38 - protein, 32 aa, Artificial Sequence
  Synthetic sequence
RFTISRDNSKNTLYLQMNSLRAEDTAVYYCAR

SEQ ID NO: 39 - protein, 11 aa, Artificial Sequence
  Synthetic sequence
WGQGTMVTVSS

SEQ ID NO: 40 - protein, 22 aa, Artificial Sequence
  Synthetic sequence
QSVLTQPPSASGTPGQRVTISC

SEQ ID NO: 41 - protein, 15 aa, Artificial Sequence
  Synthetic sequence
WYQQLPGTAPKLLIS

SEQ ID NO: 42 - protein, 32 aa, Artificial Sequence
  Synthetic sequence
GVPDRFSGSKSGTSASLAISGLRSEDEADYYC

SEQ ID NO: 43 - protein, 10 aa, Artificial Sequence
  Synthetic sequence
FGGGTKLTVL

SEQ ID NO: 44 - protein, 22 aa, Artificial Sequence
  Synthetic sequence
QYELTQPPSASGTPGQRVTMSC

SEQ ID NO: 45 - protein, 330 aa, Homo sapiens [sequence omitted]

SEQ ID NO: 46 - protein, 330 aa, Artificial Sequence
  Synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 648 bp, Artificial Sequence
  Synthetic sequence
cagtacgaattgactcagccaccctcagcgtctgggacccccgggcagagtgtcaccatgtcttgttctgggagcagctccaatatcggacttaattatgtatcctggtaccagcacctcccaggaacggcccccaaactcctcatctctaggaataatcagcggccctcaggggtccctgaccgattctctggctccaagtctggcacctcagcctccctggccatcagtgggctccggtccgaagatgaggctgattattattgtgcagcatgggatgacagcctgagtggtgaggtattcggcggagggaccaagctgaccgtcctcggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctgcagaatgctct

SEQ ID NO: 48 - DNA, 356 bp, Artificial Sequence
  Synthetic sequence
gaagttcaattgttagagtctggtggcggtcttgttcagcctggtggttctttacgtctttcttgcgctgcttccggattcactttctcttattactatatgcagtgggttcgccaagctcctggtaaaggtttggagtgggtttcttatatcggttcttctggtggcgttactaattatgctgactccgttaaaggtcgcttcactatctctagagacaactctaagaatactctctacttgcagatgaacagcttaagggctgaggacacggccgtgtattactgtgcgagagtaggtcttggggatgcttttgatatctggggccaagggacaatggtcaccgtctcaagcgc

(19) United States
(12) Patent Application Publication
(10) Pub. No.: US 2017/0190788 A1
(43) Pub. Date: Jul. 6, 2017

(54) USES OF ANTI-HER3 ANTIBODIES FOR TREATING CANCER

(71) Applicants: Theresa Marie LAVALLEE, Rockville, MD (US); Carolyn Fowler SIDOR, Chapel Hill, NC (US); Diego ALVARADO, Madison, CT (US); BULLDOG PHARMACEUTICALS, INC., Road Town (VG)

(72) Inventors: Theresa Marie LAVALLEE, Rockville, MD (US); Carolyn Fowler SIDOR, Chapel Hill, NC (US); Diego ALVARADO, Madison, CT (US)

(73) Assignee: BULLDOG PHARMACEUTICALS, INC., Road Town (VG)

(21) Appl. No.: 15/510,574

(22) PCT Filed: Sep. 10, 2015
(86) PCT No.: PCT/US15/49413

§ 371 Date: Mar. 10, 2017

Related U.S. Application Data
(60) Provisional application No. 62/049,316, filed on Sep. 11, 2014.
(60) Provisional application No. 62/066,207, filed on Oct. 20, 2014.
(60) Provisional application No. 62/080,947, filed on Nov. 17, 2014.
(60) Provisional application No. 62/142,760, filed on Apr. 3, 2015.
(60) Provisional application No. 62/175,120, filed on Jun. 12, 2015.
(60) Provisional application No. 62/215,275, filed on Sep. 8, 2015.

(51) Int. Cl. C07K 16/32; A61K 39/395; A61K 31/519; A61K 51/10; C07K 16/28; A61K 31/517; A61K 31/506; C07K 16/30; A61K 31/437

(52) U.S. Cl. CPC C07K 16/32; C07K 16/30; A61K 39/3955; A61K 31/519; C07K 16/3015; C07K 16/3023; C07K 16/3046; C07K 16/3053; A61K 31/437; C07K 16/2863; A61K 31/517; A61K 31/506; A61K 51/1045; C07K 2317/56; C07K 2317/52; C07K 2317/76; C07K 2317/567; C07K 2317/565; C07K 2317/24; C07K 2317/21; C07K 2317/31; C07K 2317/54; C07K 2317/55; C07K 2317/622; C07K 2317/624; C07K 2317/33; A61K 2039/54

(57) ABSTRACT

Provided herein are methods of treating cancer comprising administering antibodies and antigen-binding fragments thereof that bind the extracellular domain of the HER3 receptor and inhibit various HER3 receptor related functions via ligand-dependent and/or ligand-independent mechanisms, and dosing regimens for related monotherapies and combination therapies.

This application claims the benefit of U.S. Provisional Application No. 62/049,316, filed Sep. 11, 2014, U.S. Provisional Application No. 62/066,207, filed Oct. 20, 2014, U.S. Provisional Application No. 62/080,947, filed Nov. 17, 2014, U.S. Provisional Application No. 62/142,760, filed Apr. 3, 2015, U.S. Provisional Application No. 62/175,120, filed Jun. 12, 2015, and U.S. Provisional Application No. 62/215,275, filed Sep. 8, 2015, each of which is hereby incorporated by reference in its entirety.

### REFERENCE TO SEQUENCE LISTING SUBMITTED ELECTRONICALLY

This application incorporates by reference a Sequence Listing submitted with this application as text file entitled “Sequence_Listing_12638-130-228.TXT” created on Sep. 10, 2015 and having a size of 32,768 bytes.

### FIELD

Provided herein are uses of anti-HER3 antibodies for the treatment of cancer, and dosing regimens for related monotherapy and combination therapy.

### BACKGROUND

The human epidermal growth factor receptor 3 (HER3, also known as Erbb3) is a receptor protein tyrosine and belongs to the epidermal growth factor receptor (EGFR) EGFR/HER subfamily of receptor protein tyrosine kinases (RTK), consisting of EGFR (HER1/Erbb1), HER2/Erbb2, HER3/Erbb3 and HER4/Erbb4. EGFR and HER2 are among the most well-established oncogenic RTKs driving the tumorigenesis of multiple types of solid tumors, including major categories such as breast, colorectal, and lung cancers. The tyrosine kinase activities of EGFR and HER2 have been shown to be essential for their oncogenic activities.

Like the prototypical EGFR, the transmembrane receptor HER3 consists of an extracellular ligand-binding domain (ECD), a dimerization domain within the ECD, an transmembrane domain, and intracellular protein tyrosine kinase domain (TKD) and a C-terminal phosphorylation domain (see, e.g., Kim et al. (1998), Biochem. J. 334, 189-195; Roepstorff et al. (2008) Histochem. Cell Biol. 129, 563-578).

The ligand Heregulin (HRG) binds to the extracellular domain of HER3 and activates the receptor-mediated signaling pathway by promoting dimerization with other EGFR family members (e.g., other HER receptors) and transphosphorylation of its intracellular domain. HER3 has been shown to lack detectable tyrosine kinase activity, likely due to a non-conservative replacement of certain key residues in the tyrosine kinase domain. Therefore, a consequence of this kinase-deficiency, HER3 needs to form hetero-dimers with other RTKs, especially EGFR and HER2, to undergo phosphorylation and be functionally active.

The central role for HER3 in oncogenesis is acting as a scaffolding protein to enable the maximum induction of the PI3K/AKT pathway. HER3 has been shown to contain a cluster of six C-terminal tyrosine-containing motifs that when phosphorylated, mimics the consensus PI3K/p85 binding site. Hence by forming heterodimers with HER3, the upstream onco-drivers, EGFR, HER2, cMET and FGFR2, can couple most efficiently to the PI3K/AKT pathway. Therefore, it is reasonable to expect that a loss of HER3 activity can block cancer progression in diverse systems driven by divergent RTKs. Studies have shown that HER3 siRNA knockdown in HER2-amplified breast cancer cells led to similar anti-proliferation effects as HER2 siRNA knockdown, further demonstrating the cancer's critical need for HER3.

Besides promoting tumor growth in unstressed conditions, HER3 has been found to be highly involved in conferring therapeutic resistances to many targeted drugs, including EGFR tyrosine kinase inhibitors, HER2 monoclonal antibodies such as trastuzumab, as well as small molecule inhibitors of PI3K or AKT or MEK.

HER3 has two different ways to dimerize with its partner RTKs: ligand-dependent (in the presence of HRG) or ligand-independent. In terms of HER2-HER3 dimers, it is known that in cells with low to medium HER2 expression, HER3 can only complex with HER2 after ligand-binding; in contrast, in cells with amplified HER2 (HER2 IHC 3+), they form spontaneous dimers without HRG (Junttila et al. (2009) Cancer Cell. 15(5):429-40). The dimers formed in the presence or absence of the ligand are structurally distinct as was demonstrated by an earlier study showing that trastuzumab/Herceptin® (Genentech/Roche HER2 monoclonal antibody approved for HER2 3+ breast cancers) can only disrupt the ligand-independent dimer but not the ligand-dependent dimer, whereas pertuzumab\Omnitarg® (rhuMAb 2C4, Genentech/Roche HER2 monoclonal antibody in phase 3 trials) can only disrupt the ligand-dependent dimers.

Dimer formation between HER family members expands the signaling potential of HER3 and is a means not only for signal diversification but also for signal amplification. HER3 has been shown to be phosphorylated in a variety of cellular contexts. For example, HER3 is constitutively phosphorylated on tyrosine residues in a subset of human breast cancer cells overexpressing HER3 (see, e.g., Kraus et al. (1993) Proc. Natl. Acad. Sci. USA 90, 2900-2904; Kim et al. (1998), Biochem. J. 334, 189-195; Schaefer et al. (2004) Cancer Res. 64, 3395-3405; Schaefer et al. (2006) Neoplasia 8, 612-622). Accordingly, therapies that effectively interfere with HER3 phosphorylation are desirable.

In addition, HER3 has been found to be overexpressed and/or overactivated in several types of cancers such as breast cancer, ovarian cancer, prostate cancer, liver cancer, kidney and urinary bladder cancers, pancreatic cancers, brain cancers, hematopoietic neoplasms, retinoblastomas, melanomas, colorectal cancers, gastric cancers, head and neck cancers, lung cancer, etc. (see, e.g., Sithanandam & Anderson (2008) Cancer Gene Ther. 15, 413-448). In general, HER3 is frequently activated in EGFR, HER2, C-Met, and FGFRII-expressing cancers.

A correlation between the expression of HER2/HER3 and the progression from a non-invasive to an invasive stage has been shown (Alimandi et al., Oncogene 10, 1813-1821; DeFazio et al., Cancer 87, 487-498; Naidu et al., Br. J. Cancer 78, 1385-1390). Sustained HER3 activation of PI3K/AKT has been repetitively shown to account for tumor resistance to EGFR/HER2 inhibitors.

There is a need for improved immunotherapeutic agents that effectively inhibit HER3-mediated cell signaling that can be used for diagnosis, prognosis prediction, and treatment of a variety of cancers, as well as clinical protocols and dosing regimens to effectively use anti-HER3 antibodies for the treatment of cancer.

### BRIEF SUMMARY

The disclosure provides methods for administering anti-HER3 antibodies or antigen-binding fragments thereof, e.g., monoclonal antibodies capable of suppressing HER3 activity in both ligand-dependent and independent settings, to a patient in need thereof for preventing/protecting against or treating cancer or one or more symptoms thereof. In certain aspects, methods of treating cancer disclosed herein comprise administering affinity matured anti-HER3 antibodies or an antigen-binding fragment thereof with increased potency and extended half-life, which consequently can be administered less frequently, at an increased inter-dose interval, and in smaller dose volumes. The disclosure also provides methods of treating diseases such as cancer (e.g., melanoma, pancreatic cancer, thyroid cancer, colorectal cancer, head and neck cancer, lung cancer, breast cancer, or gastric cancer) in a human subject comprising administration of an anti-HER3 antibody (e.g., 2C2-YTE) or an antigen-binding fragment thereof, for example, alone, or in combination with another agent or with two or more agents, such as a chemotherapeutic agent (e.g., a platinum), a BRAF inhibitor (e.g., vemurafenib), a MEK inhibitor (e.g., trametinib), an EGFR inhibitor (e.g., cetuximab or erlotinib) or a HER2 inhibitor (e.g., trastuzumab), or the combination of BRAF and MEK inhibitors (e.g. the combination of dabrafenib and trametinib). In some specific aspects, a 2C2-derived YTE mutant human antibody is used.

In one aspect, the disclosure provides methods of treating cancer (e.g., melanoma, thyroid cancer, colon cancer, lung cancer, head and neck cancer, breast cancer, or gastric cancer) in a human subject comprising administering to a human subject an amount of an anti-HER3 antibody (e.g., 2C2-YTE) or an antigen-binding fragment thereof sufficient to achieve a serum concentration (or serum PK level) at or above a target serum concentration (e.g., Cₘᵢₙ or trough concentration) in the subject, for monotherapy or combination therapy, for example, in combination with a BRAF inhibitor (e.g., vemurafenib), an EGFR inhibitor (e.g., cetuximab or erlotinib) or a HER2 inhibitor (e.g., trastuzumab).

In specific aspects, disclosed herein is a method of treating cancer in a human subject in need thereof, comprising administering to the human subject an amount of an anti-HER3 antibody or antigen-binding fragment thereof which specifically binds to an epitope within the extracellular domain of HER3, wherein the anti-HER3 antibody specifically binds to the same HER3 epitope as an antibody or antigen-binding fragment thereof comprising the heavy chain variable region (VH) and light chain variable region (VL) of CL16 or 2C2 or competitively inhibits HER3 binding by an antibody or antigen-binding fragment thereof comprising the VH and VL of CL16 or 2C2, and wherein the amount of the anti-HER3 antibody or antigen-binding fragment thereof is sufficient to achieve:
(i) a Cₘᵢₙ of 50 μg/mL or greater antibody serum concentration; or
(ii) antibody serum concentration of 50 μg/mL or greater, for example, throughout the dosing interval (e.g., approximately 7 days, 14 days, 21 days, or 28 days). In one embodiment, the dosing interval is 21 days. In a particular aspect, the VH and VL of CL16 comprise SEQ ID NOs: 2 and 1, respectively, and the VH and VL of 2C2 comprise SEQ ID NOs: 2 and 3, respectively.

In a specific aspect, provided herein is a method of treating cancer in a human subject in need thereof, comprising administering to the subject an amount of an anti-HER3 antibody or antigen-binding fragment thereof which specifically binds to an epitope within the extracellular domain of HER3, wherein the anti-HER3 antibody or antigen binding fragment comprises an antibody VL of SEQ ID NO:3, an antibody VH of SEQ ID NO: 2, and an IgG constant region, for example, an IgG1 constant region, e.g., an IgG1 constant region of SEQ ID 46, and wherein the amount of the anti-HER3 antibody or antigen-binding fragment thereof is sufficient to achieve
(i) a Cₘᵢₙ of 50 μg/mL or greater antibody serum concentration; or
(ii) antibody serum concentration of 50 μg/mL or greater, for example, throughout the dosing interval (e.g., approximately 7 days, 14 days, 21 days, or 28 days). In a particular aspect, the human IgG constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

In certain embodiments, the amount anti-HER3 antibody or antigen-binding fragment thereof is sufficient to achieve a Cₘᵢₙ of 50 μg/mL or greater antibody serum concentration. In other embodiments, the amount of the anti-HER3 antibody or antigen-binding fragment thereof is sufficient to achieve antibody serum concentration of 50 μg/mL or greater throughout the dosing interval (e.g., approximately 7 days, 14 days, 21 days, or 28 days). In one embodiment the dosing interval is 21 days. In certain embodiments, the cancer is a head and neck cancer. In certain embodiments, the cancer is a squamous cell carcinoma head and neck cancer. In particular embodiments, the cancer is a thyroid cancer. In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a particular aspect, the amount of the anti-HER3 antibody or antigen-binding fragment thereof administered is sufficient to achieve at least one of the following over a period of 1 week (e.g., 7 days), 2 weeks (e.g., 14 days), 3 weeks (e.g., 21 days) or 4 weeks (e.g., 28 days):
(i) a Cₘᵢₙ of 50 μg/mL or greater antibody serum concentration; and
(ii) an antibody serum concentration of 50 μg/mL or greater, for example, throughout the dosing interval (e.g., approximately 7 days, 14 days, 21 days, or 28 days). In one embodiment the dosing interval is 21 days. In a specific aspect, the anti-HER3 antibody or antigen-binding fragment thereof administered is sufficient to achieve (i) a Cₘᵢₙ of 50 μg/mL or greater antibody serum concentration, and/or (ii) an antibody serum concentration of 50 μg/mL or greater throughout the dosing interval, e.g., throughout a period of 3 weeks. In a specific aspect, the anti-HER3 antibody or antigen-binding fragment thereof administered is sufficient to achieve (i) a Cₘᵢₙ of 50 μg/mL or greater antibody serum concentration, or (ii) an antibody serum concentration of 50 μg/mL or greater throughout the dosing interval, e.g., throughout a period of at least 2 weeks. In a specific aspect, the anti-HER3 antibody or antigen-binding fragment thereof administered is sufficient to achieve (i) a Cₘᵢₙ of 50 μg/mL or greater antibody serum concentration, or (ii) an antibody serum concentration of 50 μg/mL or greater throughout the dosing interval, e.g., throughout a period of at least 6 days, at least 7 days, at least 8 days, at least 12 days, at least 13 days, at least 14 days, at least 15, days, at least 19 days, or at least 20 days, following antibody administration. In a specific aspect, the amount of the anti-HER3 antibody or antigen-binding fragment thereof administered is 5 mg/kg, 10 mg/kg, 15 mg/kg, or 20 mg/kg every week, every 2 weeks, every 3 weeks or every 4 weeks. In a specific aspect, the amount of the anti-HER3 antibody or antigen-binding fragment thereof administered is 5 mg/kg every week. In a specific aspect, the amount of the anti-HER3 antibody or antigen-binding fragment thereof administered is 10 mg/kg every week. In a specific aspect, the amount of the anti-HER3 antibody or antigen-binding fragment thereof administered is 15 mg/kg every week. In a specific aspect, the amount of the anti-HER3 antibody or antigen-binding fragment thereof administered is 20 mg/kg every week. In a specific aspect, the amount of the anti-HER3 antibody or antigen-binding fragment thereof administered is 5 mg/kg every two weeks. In a specific aspect, the amount of the anti-HER3 antibody or antigen-binding fragment thereof administered is 10 mg/kg every two weeks. In a specific aspect, the amount of the anti-HER3 antibody or antigen-binding fragment thereof administered is 15 mg/kg every two weeks. In a specific aspect, the amount of the anti-HER3 antibody or antigen-binding fragment thereof administered is 20 mg/kg every two weeks. In a specific aspect, the amount of the anti-HER3 antibody or antigen-binding fragment thereof administered is 5 mg/kg every three weeks. In a specific aspect, the amount of the anti-HER3 antibody or antigen-binding fragment thereof administered is 10 mg/kg every three weeks. In a specific aspect, the amount of the anti-HER3 antibody or antigen-binding fragment thereof administered is 15 mg/kg every three weeks. In a specific aspect, the amount of the anti-HER3 antibody or antigen-binding fragment thereof administered is 20 mg/kg every three weeks. In a specific aspect, the amount of the anti-HER3 antibody or antigen-binding fragment thereof administered is 5 mg/kg every four weeks. In a specific aspect, the amount of the anti-HER3 antibody or antigen-binding fragment thereof administered is 10 mg/kg every four weeks. In a specific aspect, the amount of the anti-HER3 antibody or antigen-binding fragment thereof administered is 15 mg/kg every four weeks. In a specific aspect, the amount of the anti-HER3 antibody or antigen-binding fragment thereof administered is 20 mg/kg every four weeks. In one aspect, the amount of the anti-HER3 antibody or antigen-binding fragment thereof administered is 5 mg/kg every week or 10 mg/kg every two weeks. In a particular aspect, the amount of the anti-HER3 antibody or antigen-binding fragment thereof administered is 20 mg/kg every 3 weeks. In one aspect, the amount of the anti-HER3 antibody or antigen-binding fragment thereof administered is a fixed dose of 1200 mg every week, every 2 weeks, every 3 weeks, or every 4 weeks. In a specific aspect, the amount of the anti-HER3 antibody or antigen-binding fragment thereof administered is 1200 mg every 4 weeks. In a specific aspect, the amount of the anti-HER3 antibody or antigen-binding fragment thereof administered is 1200 mg every 3 weeks. In a specific aspect, the amount of the anti-HER3 antibody or antigen-binding fragment thereof administered is 1200 mg every 2 weeks. In a specific aspect, the amount of the anti-HER3 antibody or antigen-binding fragment thereof administered is 1200 mg every week. In a particular aspect, the amount of the anti-HER3 antibody or antigen-binding fragment thereof administered is 5 mg/kg weekly or 500 mg weekly. In a particular aspect, the amount of the anti-HER3 antibody or antigen-binding fragment thereof administered is 500 mg weekly. In a particular aspect, the amount of the anti-HER3 antibody or antigen-binding fragment thereof administered is 5 mg/kg every 2 weeks or 500 mg every 2 weeks. In a particular aspect, the amount of the anti-HER3 antibody or antigen-binding fragment thereof administered is 5 mg/kg every 10 or 11 days or 500 mg every 10 or 11 days. In a certain aspect, the amount of the anti-HER3 antibody or antigen-binding fragment thereof administered is 10 mg/kg every 2 weeks or 3 weeks or 1000 mg every 2 weeks or 3 weeks. In a certain aspect, the amount of the anti-HER3 antibody or antigen-binding fragment thereof administered is 1000 mg every 2 weeks. In a certain aspect, the amount of the anti-HER3 antibody or antigen-binding fragment thereof administered is 1000 mg every 3 weeks. In one aspect, the amount of the anti-HER3 antibody or antigen-binding fragment thereof administered is sufficient to achieve an antibody serum concentration in the range of 50 μg/mL to 500 μg/mL, for example over a period of 1 week, 2 weeks, 3 weeks or 4 weeks between each administration. In a certain aspect, the amount of the anti-HER3 antibody or antigen-binding fragment thereof administered is sufficient to achieve an antibody serum concentration in the range of 50 μg/mL to 250 μg/mL, for example over a period of 3 weeks between each administration. In one aspect, the amount of the anti-HER3 antibody or antigen-binding fragment thereof administered is sufficient to achieve an antibody serum concentration in the range of 50 μg/mL to 500 μg/mL, for example over a period of 3 weeks between each administration. In a certain aspect, the amount of the anti-HER3 antibody or antigen-binding fragment thereof administered is sufficient to achieve an antibody serum concentration in the range of 50 μg/mL to 250 μg/mL, for example over a period of 3 weeks between each administration. In a particular aspect, the human subject is administered a loading dose (e.g., a first or initial dose) of the anti-HER3 antibody or antigen-binding fragment thereof that is higher than a subsequent dose of the anti-HER3 antibody or antigen-binding fragment thereof. In a certain aspect, the loading dose (e.g., a first or initial dose) is administered 1 week, 2 weeks, 3 weeks, or 4 weeks prior to the subsequent dose of the anti-HER3 antibody or antigen-binding fragment thereof. In a certain aspect, the loading dose (e.g., a first or initial dose) is administered 1 week, 2 weeks, or 3 weeks prior to the subsequent dose of the anti-HER3 antibody or antigen-binding fragment thereof. In one aspect, the loading dose (e.g., a first or initial dose) is at least 1.5 fold, at least 2 fold, at least 3 fold, at least 4 fold, or at least 5 fold higher than a subsequent dose of anti-HER3 or an antigen-binding fragment thereof. In a particular aspect, the subsequent dose of the anti-HER3 antibody or antigen-binding fragment thereof is 5 mg/kg, 10 mg/kg, 15 mg/kg, or 20 mg/kg every week, every 2 weeks, every 3 weeks or every 4 weeks. In a particular aspect, the subsequent dose of the anti-HER3 antibody or antigen-binding fragment thereof is 5 mg/kg every week, 10 mg/kg every 2 weeks, 15 mg/kg every 3 weeks, or 20 mg/kg every 3 weeks. In a specific aspect, the subsequent dose of the anti-HER3 antibody or antigen-binding fragment thereof administered is 5 mg/kg every week. In a specific aspect, the subsequent dose of the anti-HER3 antibody or antigen-binding fragment thereof administered is 10 mg/kg every week. In a specific aspect, the subsequent dose of the anti-HER3 antibody or antigen-binding fragment thereof administered is 15 mg/kg every week. In a specific aspect, the subsequent dose of the anti-HER3 antibody or antigen-binding fragment thereof administered is 20 mg/kg every week. In a specific aspect, the subsequent dose of the anti-HER3 antibody or antigen-binding fragment thereof administered is 5 mg/kg every two weeks. In a specific aspect, the subsequent dose of the anti-HER3 antibody or antigen-binding fragment thereof administered is 10 mg/kg every two weeks. In a specific aspect, the subsequent dose of the anti-HER3 antibody or antigen-binding fragment thereof administered is 15 mg/kg every two weeks. In a specific aspect, the subsequent dose of the anti-HER3 antibody or antigen-binding fragment thereof administered is 20 mg/kg every two weeks. In a specific aspect, the subsequent dose of the anti-HER3 antibody or antigen-binding fragment thereof administered is 5 mg/kg every three weeks. In a specific aspect, the subsequent dose of the anti-HER3 antibody or antigen-binding fragment thereof administered is 10 mg/kg every three weeks. In a specific aspect, the subsequent dose of the anti-HER3 antibody or antigen-binding fragment thereof administered is 15 mg/kg every three weeks. In a specific aspect, the subsequent dose of the anti-HER3 antibody or antigen-binding fragment thereof administered is 20 mg/kg every three weeks. In a specific aspect, the subsequent dose of the anti-HER3 antibody or antigen-binding fragment thereof administered is 5 mg/kg every four weeks. In a specific aspect, the subsequent dose of the anti-HER3 antibody or antigen-binding fragment thereof administered is 10 mg/kg every four weeks. In a specific aspect, the subsequent dose of the anti-HER3 antibody or antigen-binding fragment thereof administered is 15 mg/kg every four weeks. In a specific aspect, the subsequent dose of the anti-HER3 antibody or antigen-binding fragment thereof administered is 20 mg/kg every four weeks. In a certain aspect, the anti-HER3 antibody or antigen-binding fragment thereof is administered intravenously. In one aspect, the anti-HER3 antibody or antigen-binding fragment thereof is administered as a 60-minute intravenous infusion.

In one aspect, the anti-HER3 antibody or antigen-binding fragment thereof comprises an antibody VL, wherein the VL comprises the amino acid sequence:

[FW₁]X₁GSX₂SNIGLNYVS[FW₂]RNNQRPS[FW₃]AAWDDX₃X₄X₅GEX₆
[FW₄]

wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A). In a certain aspect, FW1 comprises SEQ ID NO: 40 or 44, FW2 comprises SEQ ID NO: 41, FW3 comprises SEQ ID NO: 42, and FW4 comprises SEQ ID NO: 43. In a particular aspect, the anti-HER3 antibody or antigen-binding fragment thereof comprises an antibody VH, wherein the VH comprises the amino acid sequence:

[FW₅]YYYMQ[FW₆]X₇IGSSGGVTNYADSVKG[FW₇]VGLGDAFDI[FW₈]

wherein [FW5], [FW6], [FW7] and [FW8] represent VH framework regions, and wherein X₇ represents amino acid residues Tyrosine (Y), Isoleucine (I) or Valine (V). In a specific aspect, FW5 comprises SEQ ID NO: 36, FW6 comprises SEQ ID NO: 37, FW7 comprises SEQ ID NO: 38 and FW8 comprises SEQ ID NO: 39. In a certain aspect, the anti-HER3 antibody or antigen-binding fragment comprises an antibody VL and an antibody VH, wherein the VL comprises the amino acid sequence:

[FW₁]X₁GSX₂SNIGLNYVS[FW₂]RNNQRPS[FW₃]AAWDDX₃X₄X₅GEX₆
[FW₄]

wherein [FW1], [FW2], [FW3] and [FW4] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and

wherein the VH comprises the amino acid sequence:

[FW₅]YYYMQ[FW₆]X₇IGSSGGVTNYADSVKG[FW₇]VGLGDAFDI[FW₈]

wherein [FW5], [FW6], [FW7] and [FW8] represent VH framework regions, and wherein

X₇ represents amino acid residues Tyrosine (Y), Isoleucine (I) or Valine (V). In a particular aspect, FW1 comprises SEQ ID NO: 40 or 44, FW2 comprises SEQ ID NO: 41, FW3 comprises SEQ ID NO: 42, FW4 comprises SEQ ID NO: 43, FW5 comprises SEQ ID NO: 36, FW6 comprises SEQ ID NO: 37, FW7 comprises SEQ ID NO: 38, and FW8 comprises SEQ ID NO: 39. In a specific aspect, the anti-HER3 antibody or antigen-binding fragment thereof comprises an antibody VL, wherein the VL comprises (i) a VL complementarity determining region-1 (VL-CDR1) amino acid sequence identical to, or identical except for four, three, two or one amino acid substitutions to: SEQ ID NO: 18, SEQ ID NO: 19, or SEQ ID NO: 20; (ii) a VL complementarity determining region-2 (VL-CDR2) amino acid sequence identical to, or identical except for four, three, two or one amino acid substitutions to SEQ ID NO: 21; and (iii) a complementarity determining region-3 (VL-CDR3) amino acid sequence identical to, or identical except for four, three, two, or one amino acid substitutions to: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. In a certain aspect, the anti-HER3 antibody or antigen-binding fragment thereof comprises an antibody VH, wherein the VH comprises (i) a complementarity determining region-1 (VH-CDR1) amino acid sequence identical to, or identical except for four, three, two, or one amino acid substitutions to SEQ ID NO: 31; (ii) a complementarity determining region-2 (VH-CDR2) amino acid sequence identical to, or identical except for four, three, two, or one amino acid substitutions to: SEQ ID NO: 32, SEQ ID NO: 33, or SEQ ID NO: 34; and (iii) a complementarity determining region-3 (VH-CDR3) amino acid sequence identical to, or identical except for four, three, two, or one amino acid substitutions to SEQ ID NO: 35. In a certain aspect, the anti-HER3 antibody or antigen-binding fragment thereof comprises an antibody VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 18, 21 and 22, SEQ ID NOs: 18, 21, and 26, SEQ ID NOs: 18, 21, and 27, SEQ ID NOs: 20, 21, and 22, SEQ ID NOs: 19, 21, and 22, SEQ ID NOs: 18, 21, and 25, SEQ ID NOs: 18, 21, and 28, SEQ ID NOs: 18, 21, and 29, SEQ ID NOs: 18, 21, and 30, SEQ ID NOs: 18, 21, and 23, SEQ ID NOs: 19, 21, and 23, SEQ ID NOs: 20, 21, and 23, SEQ ID NOs: 18, 21, and 24, or SEQ ID NOs: 18, 21, and 25, respectively. In a particular aspect, the anti-HER3 antibody or antigen-binding fragment thereof comprises an antibody VH, wherein the VH comprises VH-CDR1, VH-CDR2, and VH-CDR3 amino acid sequences identical to, or identical except for four, three, two, or one amino acid substitutions in one or more of the VH-CDRS to: SEQ ID NOs: 31, 32 and 35, SEQ ID NOs: 31, 33, and 35, or SEQ ID NOs: 31, 34, and 35, respectively. In a specific aspect, the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively. In one aspect, the anti-HER3 antibody or antigen-binding fragment comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17. In one aspect, the anti-HER3 antibody or antigen-binding fragment comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, wherein the VL CDRs are identical to those of the reference amino acid sequence. In one aspect, the anti-HER3 antibody or antigen-binding fragment comprises an antibody VL and an antibody VH, wherein the VL comprises an amino acid sequence at least about 80% to about 90% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17. In one aspect, the anti-HER3 antibody or antigen-binding fragment comprises an antibody VL and an antibody VH, wherein the VL comprises an amino acid sequence at least about 80% to about 90% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, wherein the VL CDRs are identical to those of the reference amino acid sequence. In a particular aspect, the anti-HER3 antibody or antigen-binding fragment comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13. In a particular aspect, the anti-HER3 antibody or antigen-binding fragment comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13, wherein the VH CDRs are identical to those of the reference amino acid sequence. In a particular aspect, the anti-HER3 antibody or antigen-binding fragment comprises an antibody VL and an antibody VH, wherein the VH comprises an amino acid sequence at least about 80% to about 90% identical to a reference amino acid sequence selected from the group consisting of SEQ ID NO: 2, SEQ ID NO: 12 and SEQ ID NO: 13. In a particular aspect, the anti-HER3 antibody or antigen-binding fragment comprises an antibody VL and an antibody VH, wherein the VH comprises an amino acid sequence at least about 80% to about 90% identical to a reference amino acid sequence selected from the group consisting of SEQ ID NO: 2, SEQ ID NO: 12 and SEQ ID NO: 13, wherein the VH CDRs are identical to those of the reference amino acid sequence. In a specific aspect, the anti-HER3 antibody or antigen-binding fragment comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13. In a specific aspect, the anti-HER3 antibody or antigen-binding fragment comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, wherein the VL CDRs are identical to those of the reference amino acid sequence, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13, wherein the VH CDRs are identical to those of the reference amino acid sequence. In a specific aspect, the anti-HER3 antibody or antigen-binding fragment comprises a VL comprising a sequence at least about 80% to about 90% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, wherein the VL CDRs are identical to those of the reference amino acid sequence, and wherein the anti-HER3 antibody or antigen-binding fragment comprises a VH comprising a sequence at least about 80% to about 90% identical to a reference amino acid sequence selected from the group consisting of SEQ ID NO: 2, SEQ ID NO: 12 and SEQ ID NO: 13, wherein the VH CDRs are identical to those of the reference amino acid sequence. In one aspect, the anti-HER3 antibody or antigen-binding fragment thereof comprises a VL comprising the VL consensus sequence provided in Table 3 and a VH comprising the VH consensus sequence provided in Table 3. In a certain aspect, the anti-HER3 antibody or antigen-binding fragment comprises a VL comprising a sequence that is at least 80%, at least 85%, at least 90%, or at least 95% identical to SEQ ID NO: 3 and a VH comprising a sequence that is at least 80%, at least 85%, at least 90%, or at least 95% identical to SEQ ID NO: 2. In a certain aspect, the anti-HER3 antibody or antigen-binding fragment comprises a VL comprising a sequence that is at least 80%, at least 85%, at least 90%, or at least 95% identical SEQ ID NO: 3 and a VH comprising a sequence that is at least 80%, at least 85%, at least 90%, or at least 95% identical to SEQ ID NO: 2, wherein the VL CDRs are identical to those of SEQ ID NO: 3 and the VH CDRs are identical to those of SEQ ID NO: 2. In a certain aspect, the anti-HER3 antibody or antigen-binding fragment comprises a VL comprising SEQ ID NO: 3 and a VH comprising SEQ ID NO: 2. In a specific aspect, the anti-HER3 antibody or antigen-binding fragment comprises a heavy chain constant region or fragment thereof. In a particular aspect, the heavy chain constant region or fragment thereof is an IgG constant region. In a certain aspect, the IgG constant region is selected from an IgG1 constant region, an IgG2 constant region, an IgG3 constant region and an IgG4 constant region. In one aspect, the IgG constant region is an IgG1 constant region. In a particular aspect, the anti-HER3 antibody or antigen-binding fragment comprises a light chain constant region selected from the group consisting of a human kappa constant region and a human lambda constant region. In one aspect, the IgG constant domain comprises one or more amino acid substitutions relative to a wild-type IgG constant domain wherein the modified IgG has an increased half-life compared to the half-life of an IgG having the wild-type IgG constant domain. In a certain aspect, the IgG constant domain comprises one or more amino acid substitutions of amino acid residues at positions 251-257, 285-290, 308-314, 385-389, and 428-436, wherein the numbering is according to the EU index as set forth in Kabat. In a particular aspect, at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions,
   wherein the numbering is according to the EU index as set forth in Kabat. In a specific aspect, the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
   wherein the numbering is according to the EU index as set forth in Kabat. In a specific aspect, the amino acid at position 434 is substituted with an amino acid selected from the group consisting of Tryptophan (W), Methionine (M), Tyrosine (Y), and Serine (S), and wherein the numbering is according to the EU index as set forth in Kabat. In one aspect, the amino acid at position 428 is substituted with an amino acid selected from the group consisting of Threonine (T), Leucine (L), Phenylalanine (F), and Serine (S), and wherein the numbering is according to the EU index as set forth in Kabat. In a particular aspect, the amino acid at position 257 is substituted with Leucine (L), and the amino acid at Kabat position 434 is substituted with Tyrosine (Y), and wherein the numbering is according to the EU index as set forth in Kabat. In a certain aspect, the amino acid at Kabat position 428 is substituted with Leucine (L), and the amino acid at Kabat position 434 is substituted with Serine (S).

In a certain aspect, the antibody used in the methods provided herein is a fully human antibody, a humanized antibody, a chimeric antibody, a monoclonal antibody, a polyclonal antibody, a recombinant antibody, a multispecific antibody, or an antigen-binding fragment thereof. In one aspect, the antigen-binding fragment is Fv, Fab, F(ab′)2, Fab′, dsFv, scFv, and sc(Fv)2. In a specific embodiment, the anti-HER3 antibody or antigen-binding fragment is conjugated to at least one heterologous agent.

In specific aspects, disclosed herein is a method of treating a human papillomavirus (HPV) positive head and neck cancer in a human subject in need thereof, comprising administering to the human subject a therapeutically effective amount of an anti-HER3 antibody (e.g., 2C2-YTE) or an antigen-binding fragment thereof. In a specific aspect, disclosed herein is a method of treating an HPV positive head and neck cancer in a human subject in need thereof, comprising administering to the human subject a therapeutically effective amount of an anti-HER3 antibody (e.g., 2C2-YTE). In specific aspects, the HPV positive head and neck cancer (e.g. SCCHN) is a cancer of the oral cavity, hypopharynx, oropharynx, rynopharynx, or larynx. In more specific embodiments, the HPV positive head and neck cancer is an oropharyngeal cancer. In a specific aspect, a method of treating HPV positive head and neck cancer described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV positive head and neck cancer. In a certain aspect, the head and neck cancer is EGFR expressing head and neck cancer.

In specific aspects, disclosed herein is a method of treating an HPV negative head and neck cancer in a human subject in need thereof, comprising administering to the human subject a therapeutically effective amount of an anti-HER3 antibody (e.g., 2C2-YTE) or an antigen-binding fragment thereof. In a specific aspect, disclosed herein is a method of treating an HPV negative head and neck cancer in a human subject in need thereof, comprising administering to the human subject a therapeutically effective amount of an anti-HER3 antibody (e.g., 2C2-YTE). In specific aspects, the HPV positive head and neck cancer (e.g. SCCHN) is a cancer of the oral cavity, hypopharynx, oropharynx, rynopharynx, or larynx. In a specific aspect, a method of treating HPV negative head and neck cancer described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV negative head and neck cancer. In a certain aspect, the head and neck cancer is EGFR expressing head and neck cancer.

In specific aspects, disclosed herein is a method of treating a human papillomavirus (HPV) positive squamous cell carcinoma of the head and neck (SCCHN) in a human subject in need thereof, comprising administering to the human subject a therapeutically effective amount of an anti-HER3 antibody (e.g., 2C2-YTE) or an antigen-binding fragment thereof. In a specific aspect, disclosed herein is a method of treating an HPV positive SCCHN in a human subject in need thereof, comprising administering to the human subject a therapeutically effective amount of an anti-HER3 antibody (e.g., 2C2-YTE). In specific aspects, the HPV positive head and neck cancer (e.g. SCCHN) is a cancer of the oral cavity, hypopharynx, oropharynx, rynopharynx, or larynx. In more specific embodiments, the HPV positive SCCHN is oropharyngeal cancer. In a specific aspect, a method of treating HPV positive SCCHN described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV positive SCCHN. In a certain aspect, the SCCHN is EGFR expressing SCCHN.

In specific aspects, disclosed herein is a method of treating an HPV negative SCCHN in a human subject in need thereof, comprising administering to the human subject a therapeutically effective amount of an anti-HER3 antibody (e.g., 2C2-YTE) or an antigen-binding fragment thereof. In a specific aspect, disclosed herein is a method of treating an HPV negative SCCHN in a human subject in need thereof, comprising administering to the human subject a therapeutically effective amount of an anti-HER3 antibody (e.g., 2C2-YTE). In specific aspects, the HPV positive head and neck cancer (e.g. SCCHN) is a cancer of the oral cavity, hypopharynx, oropharynx, rynopharynx, or larynx. In a specific aspect, a method of treating HPV negative SCCHN described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV negative SCCHN. In a certain aspect, the SCCHN is EGFR expressing SCCHN.

In a specific aspect, a method of treating cancer described herein with an anti-HER3 antibody (e.g., 2C2-YTE) or an antigen-binding fragment thereof further comprises administering to the subject a therapeutic effective amount of a MEK inhibitor, in combination with an anti-HER3 antibody (e.g., 2C2-YTE). In one aspect the MEK inhibitor is trametinib. In certain embodiments, the cancer is a head and neck cancer. In certain embodiments, the cancer is a squamous cell carcinoma head and neck cancer. In particular embodiments, the cancer is a thyroid cancer. In certain embodiments, prior to administration of the HER3 antibody, the cancer is resistant to treatment with the MEK inhibitor (e.g., trametenib). In a particular embodiment, prior to administration of the HER3 antibody, the cancer is resistant to treatment with trametinib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a particular aspect of a method of treating cancer described herein with an anti-HER3 antibody or an antigen-binding fragment thereof, the cancer is selected from the group consisting of melanoma, thyroid cancer, colon/colorectal cancer, lung cancer (e.g., non small cell lung cancer), gastric cancer, pancreatic cancer, breast cancer, and head and neck cancer (e.g., squamous cell carcinoma of the head and neck). In a particular aspect, the cancer comprises cells comprising a KRAS mutation. In a certain aspect, the KRAS mutation comprises a mutation of codon 12 of a human KRAS gene. In a specific aspect, the cancer is characterized by expression of HER3. In a specific aspect, the cancer is characterized by expression of Neuregulin 1/Heregulin 1. In a certain aspect, the cancer is characterized by a BRAF mutation.

In one aspect, a method of treating cancer described herein with an anti-HER3 antibody (e.g., 2C2-YTE) or an antigen-binding fragment thereof further comprises administering to the human subject a therapeutic effective amount of a BRAF inhibitor. In one aspect, the BRAF inhibitor is vemurafenib. In another aspect, the BRAF inhibitor is dabrafenib. In a specific aspect, the cancer comprises cells comprising a BRAF mutation. In a particular aspect, the BRAF mutation is the BRAF V600E mutation. In a particular aspect, the BRAF mutation is the BRAF V600K mutation. In a certain aspect, the BRAF inhibitor is an antibody or antigen binding fragment thereof. In certain embodiments, the cancer is a head and neck cancer. In certain embodiments, the cancer is a squamous cell carcinoma head and neck cancer. In particular embodiments, the cancer is a thyroid cancer. In certain embodiments, prior to administration of the HER3 antibody, the cancer is resistant to treatment with the BRAF inhibitor (e.g., vemurafenib or dabrafenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a particular aspect, the human subject has been diagnosed with melanoma, for example BRAF mutated (i.e., BRAF mutant) melanoma. In a certain aspect, the human subject is administered (i) the anti-HER3 antibody or antigen-binding fragment (e.g., at a dose sufficient to achieve a target serum concentration, such as a Cₘᵢₙ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) vemurafenib at a dose of 960 mg twice daily, e.g., approximately every 12 hours, starting on day 2. In one embodiment, the dose of the anti-HER3 antibody or antigen-binding fragment thereof is 15 mg/kg.

In one aspect, a method of treating cancer (e.g., melanoma) described herein with an anti-HER3 antibody (e.g., 2C2-YTE) or an antigen-binding fragment thereof further comprises administering to the human subject a therapeutic effective amount of a BRAF inhibitor such as dabrafenib. In a particular aspect, the cancer is characterized as having cancer cells having the BRAF V600E mutation or the BRAF V600K mutation. In certain embodiments, the cancer is a head and neck cancer. In certain embodiments, the cancer is a squamous cell carcinoma head and neck cancer. In particular embodiments, the cancer is a thyroid cancer.

In one aspect, a method of treating cancer (e.g., melanoma) described herein with an anti-HER3 antibody (e.g., 2C2-YTE) or an antigen-binding fragment thereof further comprises administering to the human subject a therapeutic effective amount of a combination of a BRAF inhibitor (e.g., dabrafenib or vemurafenib) and a MEK inhibitor (e.g., trametinib). In a particular aspect, the cancer, such as melanoma, is characterized as having cancer cells having the BRAF V600E mutation or the BRAF V600K mutation. In certain embodiments, prior to administration of the HER3 antibody, the cancer is resistant to treatment with the BRAF inhibitor (e.g., vemurafenib or dabrafenib) and the MEK inhibitor (e.g., trametenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In a particular embodiment, prior to administration of the HER3 antibody, the cancer is resistant to treatment with trametinib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with head and neck cancer. In a certain aspect, the head and neck cancer is EGFR expressing head and neck cancer. In one aspect, the human subject has been diagnosed with KRAS mutated negative, EGFR expressing colon cancer. In a particular aspect, the human subject is administered (i) the anti-HER3 antibody or antigen-binding fragment (e.g., at a dose sufficient to achieve a target serum concentration, such as a Cₘᵢₙ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) cetuximab at an initial loading dose of 400 mg/m² on day 2 over approximately 2 hours followed by weekly doses of 250 mg/m² over approximately 60 minutes starting on day 8 and continuing weekly. In one embodiment, the dose of the anti-HER3 antibody or antigen-binding fragment thereof is 15 mg/kg. In a particular aspect, the human subject is administered (i) the anti-HER3 antibody or antigen-binding fragment (e.g., at a dose sufficient to achieve a target serum concentration, such as a Cₘ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) cetuximab on day 1 at a dose of 400 mg/m² or 250 mg/m² weekly or at an initial loading dose of 400 mg/m² followed by a weekly dose of 250 mg/m². In one embodiment, the dose of the anti-HER3 antibody or antigen-binding fragment thereof is 15 mg/kg. In a certain aspect, the anti-HER3 antibody or antigen-binding fragment and the cetuximab are administered approximately 2 hours apart on the same day. In certain embodiments, prior to administration of the HER3 antibody, the cancer is resistant to treatment with an EGFR inhibitor (e.g., cetuximab or trastuzumab). In a particular embodiment, prior to administration of the HER3 antibody, the cancer is resistant to treatment with cetuximab. In another particular embodiment, prior to administration of the HER3 antibody, the cancer is resistant to treatment with trastuzumab. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a certain aspect, a method of treating cancer described herein with an anti-HER3 antibody (e.g., 2C2-YTE) or an antigen-binding fragment thereof further comprises administering to the human subject a therapeutically effective amount of a reversible or irreversible small molecule inhibitor of EGFR, such as erlotinib or AZD9291. In certain embodiments, the cancer is a head and neck cancer. In certain embodiments, the cancer is a squamous cell carcinoma head and neck cancer. In particular embodiments, the cancer is a thyroid cancer. In certain embodiments, prior to administration of the HER3 antibody, the cancer is resistant to treatment with the EGFR inhibitor (e.g., erlotinib or AZD9291). In a particular embodiment, prior to administration of the HER3 antibody, the cancer is resistant to treatment with erlotinib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a certain aspect, a method of treating cancer described herein with an anti-HER3 antibody (e.g., 2C2-YTE) or an antigen-binding fragment thereof further comprises administering to the human subject a therapeutically effective amount of erlotinib. In a particular aspect, the human subject has been diagnosed with lung cancer, such as non-small cell lung cancer. In certain embodiments, the cancer is a head and neck cancer. In certain embodiments, the cancer is a squamous cell carcinoma head and neck cancer. In particular embodiments, the cancer is a thyroid cancer. In a certain aspect, the human subject is administered (i) the anti-HER3 antibody or antigen-binding fragment (e.g., at a dose sufficient to achieve a target serum concentration, such as a Cₘᵢₙ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) erlotinib on day 2 at a dose of 150 mg/day orally. In one embodiment, the dose of the anti-HER3 antibody or antigen-binding fragment thereof is 15 mg/kg. In a particular aspect, the human subject is administered (i) the anti-HER3 antibody or antigen-binding fragment by intravenous (IV) infusion on day 1 and repeated every 21 days; and (ii) erlotinib on day 1 at a dose of 150 mg/day orally. In a certain aspect, the human subject is administered (i) the anti-HER3 antibody or antigen-binding fragment by intravenous (IV) infusion on day 1 and repeated every 21 days; and (ii) erlotinib on day 2 at a dose of 100 mg/day orally. In a particular aspect, the human subject is administered (i) the anti-HER3 antibody or antigen-binding fragment by intravenous (IV) infusion on day 1 and repeated every 21 days; and (ii) erlotinib on day 1 at a dose of 100 mg/day orally. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In one aspect, a method of treating cancer (e.g., colon/colorectal cancer) described herein with an anti-HER3 antibody (e.g., 2C2-YTE) or an antigen-binding fragment thereof further comprises administering to the human subject a combination of an EGFR inhibitor (e.g., cetuximab, erlotinib or AZD9291) and a MEK inhibitor (e.g., trametinib). In certain embodiments, the cancer is a head and neck cancer. In certain embodiments, the cancer is a squamous cell carcinoma head and neck cancer. In particular embodiments, the cancer is a thyroid cancer.

In a specific aspect, a method of treating cancer described herein with an anti-HER3 antibody (e.g., 2C2-YTE) or an antigen-binding fragment thereof further comprises administering to the human subject a therapeutically effective amount of a HER2 inhibitor. In a particular aspect, the HER2 inhibitor is an anti-HER2 antibody, such as trastuzumab. In a certain aspect, the human subject has been diagnosed with breast cancer, such as HER2 positive breast cancer. In one aspect, the human subject has been diagnosed with gastric cancer, such as HER2 positive gastric cancer. In a particular aspect, the human subject had failed initial therapy for advanced or metastatic disease. In a specific aspect, the human subject is administered (i) the anti-HER3 antibody (e.g., 2C2-YTE) or antigen-binding fragment (e.g., at a dose sufficient to achieve a target serum concentration, such as a Cₘᵢₙ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) trastuzumab at an initial loading dose of 4 mg/kg IV on day 2 over approximately 90 minutes followed by 2 mg/kg IV over approximately 30 minutes starting on day 8 and continuing weekly. In a certain aspect, the human subject is administered (i) the anti-HER3 antibody or antigen-binding fragment by intravenous (IV) infusion on day 1 and repeated every 21 days; and (ii) trastuzumab at a dose of 4 mg/kg or 2 mg/kg IV weekly. In one aspect, the anti-HER3 antibody or antigen-binding fragment and trastuzumab are administered a few hours apart, e.g., approximately 2 hours apart, on the same day. In a particular aspect, the human subject is administered (i) the anti-HER3 antibody or antigen-binding fragment (e.g., at a dose sufficient to achieve a target serum concentration, such as 50 μg/mL or greater, throughout the dosing interval, or at a dose of 15 mg/kg or 20 mg/kg) by intravenous (IV) infusion on day 1 and repeated every 21 days; and (ii) trastuzumab at an initial loading dose of 8 mg/kg IV on day 2 over approximately 90 minutes followed by 6 mg/kg IV over approximately 30 minutes starting on day 8 and continuing every 3 weeks. In one aspect, the human subject is administered (i) the anti-HER3 antibody or antigen-binding fragment (e.g., at a dose sufficient to achieve a target serum concentration, such as 50 μg/mL or greater, throughout the dosing interval, or at a dose of 15 mg/kg or 20 mg/kg) by intravenous (IV) infusion on day 1 and repeated every 21 days; and (ii) trastuzumab at a dose of 8 mg/kg or 6 mg/kg IV every 3 weeks. In certain embodiments, prior to administration of the HER3 antibody, the cancer is resistant to treatment with an EGFR inhibitor (e.g., cetuximab or trastuzumab). In a particular embodiment, prior to administration of the HER3 antibody, the cancer is resistant to treatment with cetuximab. In another particular embodiment, prior to administration of the HER3 antibody, the cancer is resistant to treatment with trastuzumab. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer described herein with an anti-HER3 antibody (e.g., 2C2-YTE) or an antigen-binding fragment thereof, as a monotherapy or combination therapy, further comprises measuring in a sample from the human subject the expression level (e.g., concentration) of one or more pharmacodynamic markers, such as soluble HER3, TWEAK, TWEAK receptor, Neurexophilin-1, IL-18 binding protein, ANGL4, IL-18 receptor 1, or Kallikrein 7. In a particular aspect, the circulating concentration (e.g., circulating plasma concentration in plasma) of a pharmacodynamic marker is measured.

In specific aspects, provided herein is a method of monitoring treatment or response to treatment of a human subject diagnosed with cancer to treatment with an anti-HER3 antibody (e.g., 2C2-YTE) or antigen-binding fragment thereof, as a monotherapy or combination therapy, which specifically binds to an epitope within the extracellular domain of HER3, comprising measuring the expression level (e.g., concentration) in a sample from the human subject of a pharmacodynamic marker, such as soluble HER3, TWEAK, TWEAK receptor, Neurexophilin-1, IL-18 binding protein, ANGL4, IL-18 receptor 1, or Kallikrein 7. In a particular aspect, the anti-HER3 antibody or antigen-binding fragment comprises a VL comprising SEQ ID NO: 3 and a VH comprising SEQ ID NO: 2. In a certain aspect, the pharmacodynamic marker is measured concomitantly with or shortly prior to, and after (e.g., 1 week, 2 weeks, 3 weeks, or 4 weeks), administration of the anti-HER3 antibody (e.g., 2C2-YTE) or antigen-binding fragment. In one aspect, the circulating concentration (e.g., circulating concentration in plasma) of a pharmacodynamic marker is measured, for example by ELISA. In specific aspects, changes (e.g., increase or decrease) in the concentration (e.g., circulating concentration in plasma) of a pharmacodynamic marker, such as soluble HER3, TWEAK, TWEAK receptor, Neurexophilin-1, IL-18 binding protein, ANGL4, IL-18 receptor 1, or Kallikrein 7, is indicative of treatment. In a particular aspect, an increase in soluble HER3 circulating concentration detected for example by ELISA, following treatment with the anti-HER3 antibody (e.g., 2C2-YTE) or antigen-binding fragment thereof is indicative of treatment. In a particular aspect, a decrease in soluble HER3 circulating concentration detected for example by proteomic aptamer based approaches (e.g., aptamer-based multiplexed proteomic technology, see, e.g., Gold et al., 2010, PLOS ONE, 5:e15004), following treatment with an anti-HER3 antibody (e.g., 2C2-YTE) or antigen-binding fragment thereof is indicative of treatment. In a certain aspect, an increase in circulating concentration of TWEAK or TWEAK receptor following treatment with the anti-HER3 antibody (e.g., 2C2-YTE) or antigen-binding fragment thereof is indicative of treatment. In a particular aspect, a decrease in circulating concentration of Neurexophilin-1, IL-18 binding protein, IL-18 receptor 1, ANGL4, or Kallinkrein 7 following treatment with the anti-HER3 antibody (e.g., 2C2-YTE) or antigen-binding fragment thereof is indicative of treatment.

In particular aspects, disclosed herein is a method of treating head and neck cancer (e.g., squamous cell carcinoma of the head and neck) or colon/colorectal cancer (e.g., KRAS mutated negative, EGFR expressing colon cancer) in a human subject in need thereof, comprising administering to the subject cetuximab and an anti-HER3 antibody (e.g., 2C2-YTE antibody) or antigen-binding fragment described herein in a dose of 15 mg/kg or 20 mg/kg. In one embodiment, the dose of the anti-HER3 antibody or antigen-binding fragment thereof is 15 mg/kg. In specific aspects, the anti-HER3 antibody or antigen-binding fragment thereof is administered in a dose of 15 mg/kg or 20 mg/kg every 21 days, and cetuximab is administered weekly at a dose of 400 mg/m² or 250 mg/m². In one embodiment, the dose of the anti-HER3 antibody or antigen-binding fragment thereof is 15 mg/kg. In a particular aspect, cetuximab is administered at an initial dose of 400 mg/m² followed by a weekly dose of 250 mg/m². In certain embodiments, the anti-HER3 antibody (e.g., 2C2-YTE) or an antigen-binding fragment thereof is administered before, for example 1 day before, cetuximab. In certain embodiments, the anti-HER3 antibody (e.g., 2C2-YTE) or an antigen-binding fragment thereof is administered with cetuximab on the same day. In certain embodiments, the anti-HER3 antibody (e.g., 2C2-YTE) or an antigen-binding fragment thereof is administered in combination with cetuximab on the same day but at least 1 hour apart or at least 2 hours apart. In certain embodiments, the anti-HER3 antibody (e.g., 2C2-YTE) or an antigen-binding fragment thereof is administered before, for example 1 day before, cetuximab in the first cycle, and for subsequent cycles, is administered with cetuximab on the same day. In certain embodiments, prior to administration of the HER3 antibody, the cancer is resistant to treatment with a EGFR inhibitor (e.g., cetuximab or trastuzumab). In a particular embodiment, prior to administration of the HER3 antibody, the cancer is resistant to treatment with cetuximab. In another particular embodiment, prior to administration of the HER3 antibody, the cancer is resistant to treatment with trastuzumab. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, disclosed herein is a method of treating lung cancer, such as non-small cell lung cancer, in a human subject in need thereof, comprising administering to the subject erlotinib and an anti-HER3 antibody (e.g., 2C2-YTE antibody) or antigen-binding fragment described herein in a dose of 15 mg/kg or 20 mg/kg. In one embodiment, the dose of the anti-HER3 antibody or antigen-binding fragment thereof is 15 mg/kg. In specific aspects, the anti-HER3 antibody (e.g., 2C2-YTE) or antigen-binding fragment thereof is administered in a dose of 15 mg/kg or 20 mg/kg every 21 days, and erlotinib is administered at a daily dose of 150 mg/day orally. In one embodiment, the dose of the anti-HER3 antibody or antigen-binding fragment thereof is 15 mg/kg. In specific aspects, the anti-HER3 antibody (e.g., 2C2-YTE) or antigen-binding fragment thereof is administered in a dose of 15 mg/kg or 20 mg/kg every 21 days, and erlotinib is administered at a daily dose of 100 mg/day orally. In one embodiment, the dose of the anti-HER3 antibody or antigen-binding fragment thereof is 15 mg/kg. In specific embodiment, erlotinib is administered 1 hour before or 2 hours after meals. In certain embodiments, the anti-HER3 antibody or an antigen-binding fragment thereof is administered before, for example 1 day before, the initial administration of erlotinib. In certain embodiments, the anti-HER3 antibody or an antigen-binding fragment thereof is administered with erlotinib on the same day. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, disclosed herein is a method of treating melanoma (e.g., BRAF mutated melanoma) in a human subject in need thereof, comprising administering to the subject vemurafenib and an anti-HER3 antibody (e.g., 2C2-YTE antibody) or antigen-binding fragment described herein in a dose of 15 mg/kg or 20 mg/kg. In one embodiment, the dose of the anti-HER3 antibody or antigen-binding fragment thereof is 15 mg/kg. In specific aspects, the anti-HER3 antibody or antigen-binding fragment thereof is administered in a dose of 15 mg/kg or 20 mg/kg every 21 days, and vemurafenib is administered at a dose of 960 mg twice daily, e.g., approximately every 12 hours. In one embodiment, the dose of the anti-HER3 antibody or antigen-binding fragment thereof is 15 mg/kg. In certain embodiments, the anti-HER3 antibody or an antigen-binding fragment thereof is administered before, for example 1 day before, the initial administration of vemurafenib. In certain embodiments, the anti-HER3 antibody or an antigen-binding fragment thereof is administered with vemurafenib on the same day. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, disclosed herein is a method of treating cancer such as melanoma (e.g., BRAF mutated melanoma) in a human subject in need thereof, comprising administering to the subject a MEK inhibitor, a BRAF inhibitor (e.g., dabrafenib or vemurafenib) and an anti-HER3 antibody (e.g., 2C2-YTE antibody) or antigen-binding fragment described herein in a dose of 15 mg/kg or 20 mg/kg. In one embodiment, the dose of the anti-HER3 antibody or antigen-binding fragment thereof is 15 mg/kg. In one embodiment, the anti-HER3 antibody or antigen-binding fragment thereof is administered every 3 weeks, or 21 days. In certain embodiments, prior to administration of the HER3 antibody, the cancer is resistant to treatment with the BRAF inhibitor (e.g., vemurafenib or dabrafenib) and the MEK inhibitor (e.g., trametenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In a particular embodiment, prior to administration of the HER3 antibody, the cancer is resistant to treatment with trametinib. In certain embodiments, the cancer comprises cells that overexpress a HER3 ligand, for example neuregulin, e.g., neuregulin 1.

In particular aspects, disclosed herein is a method of treating thyroid cancer (e.g., BRAF mutated thyroid cancer, or radioiodine refractory (RAIR) thyroid cancer, for example, BRAF-mutated RAIR thyroid cancer) in a human subject in need thereof, comprising administering to the subject a BRAF inhibitor, for example, dabrafenib or vemurafenib, and an anti-HER3 antibody (e.g., 2C2-YTE antibody) or antigen-binding fragment described herein. In a specific embodiment, the RAIR thyroid cancer is a papillary or follicular thyroid cancer. In particular aspects, disclosed herein is a method of treating thyroid cancer (e.g., BRAF mutated thyroid cancer, or radioiodine refractory (RAIR) thyroid cancer, for example, BRAF-mutated RAIR thyroid cancer) in a human subject in need thereof, comprising administering to the subject a BRAF inhibitor, for example, dabrafenib or vemurafenib, and an anti-HER3 antibody (e.g., 2C2-YTE antibody) or antigen-binding fragment described herein in a dose of 1000 mg, for example, in a dose of 1000 mg every 14 days. In a specific embodiment, the anti-HER3 antibody (e.g., 2C2-YTE antibody) or antigen-binding fragment thereof is administered in a dose of 1000 mg every 14 days, and the BRAF inhibitor is vemurafenib, for example, vemurafenib, administered at a dose of 960 mg twice daily, e.g., approximately every 12 hours. In certain embodiments, the anti-HER3 antibody or an antigen-binding fragment thereof is administered after a BRAF inhibitor lead-in phase, for example is approximately 1 week after initial administration of the BRAF inhibitor, e.g., dabrafenib or vemurafenib. In a particular embodiment, the human subject has radioiodine refractory disease, for example, BRAF-mutated radioiodine refractory thyroid cancer. In one embodiment, the dose of the anti-HER3 antibody or antigen-binding fragment thereof is 15 mg/kg. In one embodiment, the anti-HER3 antibody or antigen-binding fragment thereof is administered every 3 weeks, or 21 days. In certain embodiments, prior to administration of the HER3 antibody, the cancer is resistant to treatment with the BRAF inhibitor (e.g., vemurafenib or dabrafenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, disclosed herein is a method of treating radioactive iodine refractory thyroid cancer, e.g., BRAF mutant RAIR thyroid cancer, in a human subject in need thereof, comprising administering to the subject a BRAF inhibitor, for example, vemurafenib, radioactive iodine, e.g., ¹³¹I or ¹²⁵I, and an anti-HER3 antibody (e.g., 2C2-YTE antibody) or antigen-binding fragment described herein. In a specific embodiment, the RAIR thyroid cancer is a papillary or follicular thyroid cancer. In a particular embodiment, the antibody (e.g., 2C2-YTE) or antigen-binding fragment thereof is administered in a dose of 1000 mg, for example, in a dose of 1000 mg every 14 days. In another specific embodiment, the BRAF inhibitor is vemurafenib, for example vemurafenib administered at a dose of 960 mg twice daily, e.g., approximately every 12 hours. In yet another specific embodiment, the anti-HER3 antibody (e.g., 2C2-YTE) or antigen-binding fragment thereof is administered in a dose of 1000 mg, e.g., 1000 mg every 14 days, and the BRAF inhibitor is vemurafenib, for example, vemurafenib administered at a dose of 960 mg twice daily, e.g., approximately every 12 hours. In a particular embodiment, the anti-HER3 antibody (e.g., 2C2-YTE) or antigen-binding fragment thereof is first administered following a vemurafenib lead-in phase, for example, approximately 1 week after initial administration of vemurafenib. In another embodiment, the radioactive iodine, e.g., ¹³¹I or ¹²⁵I, is administered with thyroid stimulating hormone (TSH), e.g., recombinant human TSH, for example thyrotropin alpha (thyrogen), stimulation. In a specific embodiment, the radioactive iodine, e.g., ¹³¹I or ¹²⁵I, is administered after two or three doses of the anti-HER3 antibody (e.g., 2C2-YTE) or antigen-binding fragment thereof, for example, approximately 1 week following such antibody or antigen-binding fragment administration. In one embodiment, the dose of the anti-HER3 antibody or antigen-binding fragment thereof is 15 mg/kg. In one embodiment, the anti-HER3 antibody or antigen-binding fragment thereof is administered every 3 weeks, or 21 days. In certain embodiments, prior to administration of the HER3 antibody, the cancer is resistant to treatment with the BRAF inhibitor (e.g., vemurafenib or dabrafenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a particular embodiment, a TSH, e.g., thyrogen-stimulated radioiodine lesional dosimetry, e.g., ¹²⁴I PET/CT lesional dosimetry, is performed on the human subject having a radioactive iodine refractory thyroid cancer, e.g., a BRAF radioactive iodine refractory thyroid cancer before administration of BRAF inhibitor, e.g., vemurafenib, and/or the anti-HER3 antibody or antigen-binding fragment thereof, and/or subsequent to such administration(s). In a specific embodiment, a TSH, e.g., thyrogen-stimulated radioiodine lesional dosimetry, e.g., ¹²⁴I PET/CT lesional dosimetry, is performed on the human subject after two doses of the anti-HER3 antibody (e.g., 2C2-YTE) or antigen-binding fragment thereof. In a particular embodiment, treatment is continued if the human subject demonstrates that a sufficient amount of radioiodine can be delivered to at least one tumor. For example, in a specific embodiment, treatment is continued is the human subject demonstrates that ≧2,000 cGy can be delivered to at least one tumor with <300 mCi of ¹³¹I (e.g., as determined by TSH, e.g., thyrogen-stimulated radioiodine lesional dosimetry, e.g., ¹²⁴I PET/CT lesional dosimetry).

In particular aspects, disclosed herein is a method of treating thyroid cancer (e.g., BRAF mutated thyroid cancer, or radioiodine refractory (RAIR) thyroid cancer, for example, BRAF-mutated RAIR thyroid cancer) in a human subject in need thereof, comprising administering to the subject an anti-HER3 antibody (e.g., 2C2-YTE antibody) or antigen-binding fragment and a MEK inhibitor (e.g., selumetinib or trametinib) and/or a BRAF inhibitor (e.g., dabrafenib or vemurafenib). In a specific embodiment, the RAIR thyroid cancer is a papillary or follicular thyroid cancer. In a specific embodiment, such a method comprises administering to the subject an anti-HER3 antibody (e.g., 2C2-YTE antibody) or antigen-binding fragment, a MEK inhibitor (e.g., selumetinib or trametinib) and a BRAF inhibitor (e.g., dabrafenib or vemurafenib). In a more specific embodiment, such methods can further comprise administering radioactive iodine, e.g., ¹³¹I or ¹²⁵I, for example, administering the radioactive iodine after administration of any or all of the anti-HER3 antibody or antigen binding fragment thereof, MEK inhibitor or BRAF inhibitor administration has begun. In a particular embodiment, the human subject has radioiodine refractory disease. In one embodiment, the dose of the anti-HER3 antibody or antigen-binding fragment thereof is 15 mg/kg. In one embodiment, the anti-HER3 antibody or antigen-binding fragment thereof is administered every 3 weeks, or 21 days. In certain embodiments, prior to administration of the HER3 antibody, the cancer is resistant to treatment with the BRAF inhibitor (e.g., vemurafenib or dabrafenib) and the MEK inhibitor (e.g., trametenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In a particular embodiment, prior to administration of the HER3 antibody, the cancer is resistant to treatment with trametinib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, disclosed herein is a method of treating cancer in a human subject in need thereof, comprising administering to the subject a MEK inhibitor and an anti-HER3 antibody (e.g., 2C2-YTE antibody) or antigen-binding fragment described herein in a dose of 15 mg/kg or 20 mg/kg. In one embodiment, the dose of the anti-HER3 antibody or antigen-binding fragment thereof is 15 mg/kg.

In particular aspects, disclosed herein is a method of treating breast cancer, such as HER2 positive breast cancer, or gastric cancer, such as HER2 positive gastric cancer, in a human subject in need thereof, comprising administering to the subject trastuzumab and an anti-HER3 antibody (e.g., 2C2-YTE antibody) or antigen-binding fragment described herein in a dose of 15 mg/kg or 20 mg/kg. In one embodiment, the dose of the anti-HER3 antibody or antigen-binding fragment thereof is 15 mg/kg. In a particular embodiment, the human subject has failed initial therapy for advanced or metastatic disease. In specific aspects, the anti-HER3 antibody or antigen-binding fragment thereof is administered in a dose of 15 mg/kg or 20 mg/kg every 21 days, and trastuzumab is administered weekly at a dose of 4 mg/kg or 2 mg/kg intravenously. In one embodiment, the dose of the anti-HER3 antibody or antigen-binding fragment thereof is 15 mg/kg. In one embodiment, the anti-HER3 antibody or antigen-binding fragment thereof is administered every 3 weeks, or 21 days. In a particular aspect, trastuzumab is administered, for example to a HER positive breast cancer patient, at an initial dose of 4 mg/kg followed by a weekly dose of 2 mg/kg. In specific aspects, the anti-HER3 antibody or antigen-binding fragment thereof is administered in a dose of 15 mg/kg or 20 mg/kg every 21 days, and trastuzumab is administered, for example to a HER positive gastric cancer patient, at a dose of 8 mg/kg or 6 mg/kg intravenously. In one embodiment, the dose of the anti-HER3 antibody or antigen-binding fragment thereof is 15 mg/kg. In a particular aspect, trastuzumab is administered, for example to a HER positive gastric cancer patient, at an initial dose of 8 mg/kg followed by a dose of 6 mg/kg every three weeks. In certain embodiments, the anti-HER3 antibody or an antigen-binding fragment thereof is administered before, for example 1 day before, trastuzumab. In certain embodiments, the anti-HER3 antibody or an antigen-binding fragment thereof is administered in combination trastuzumab on the same day. In certain embodiments, the anti-HER3 antibody (e.g., 2C2-YTE) or an antigen-binding fragment thereof is administered in combination with trastuzumab on the same day but at least 1 hour apart or at least 2 hours apart. In certain embodiments, the anti-HER3 antibody (e.g., 2C2-YTE) or an antigen-binding fragment thereof is administered before, for example 1 day before, trastuzumab in the first cycle, and for subsequent cycles, is administered with trastuzumab on the same day. In certain embodiments, prior to administration of the HER3 antibody, the cancer is resistant to treatment with trastuzumab. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

The disclosure provides, for use in the methods described herein, an isolated binding molecule (e.g., anti-HER3 antibody such as 2C2-YTE) or antigen-binding fragment thereof which specifically binds to an epitope within the extracellular domain of HER3, wherein the binding molecule specifically binds to the same HER3 epitope as an antibody or antigen-binding fragment thereof comprising the heavy chain variable region (VH) and light chain variable region (VL) of CL16 or 2C2. Also provided, for use in the methods described herein, is an isolated binding molecule (e.g., anti-HER3 antibody such as 2C2-YTE) or antigen-binding fragment thereof which specifically binds to HER3, and competitively inhibits HER3 binding by an antibody or antigen-binding fragment thereof comprising the VH and VL of CL16 or 2C2.

The disclosure also provides, for use in the methods described herein, an isolated binding molecule (e.g., anti-HER3 antibody such as 2C2-YTE) or antigen binding fragment thereof which specifically binds to HER3 comprising an antibody VL, wherein the VL comprises the amino acid sequence:

[FW₁]X₁GSX₂SNIGLNYVS[FW₂]RNNQRPS[FW₃]AAWDDX₃X₄X₅GEX₆
[FW₄]

wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A).

Furthermore, the disclosure provides, for use in the methods described herein, an isolated binding molecule (e.g., anti-HER3 antibody such as 2C2-YTE) or antigen binding fragment thereof which specifically binds to HER3 comprising an antibody VH, wherein the VH comprises the amino acid sequence:

[FW₅]YYYMQ[FW₆]X₇IGSSGGVTNYADSVKG[FW₇]VGLGDAFDI[FW₈]

wherein [FW₅], [FW₆], [FW_{D}] and [FW₈] represent VH framework regions, and wherein

X₇ represents amino acid residues Tyrosine (Y), Isoleucine (I) or Valine (V).

The disclosure provides, for use in the methods described herein, an isolated binding molecule (e.g., anti-HER3 antibody such as 2C2-YTE) or antigen binding fragment thereof which specifically binds to HER3 comprising an antibody VL and an antibody VH, wherein the VL comprises the amino acid sequence:

[FW₁]X₁GSX₂SNIGLNYVS[FW₂]RNNQRPS[FW₃]AAWDDX₃X₄X₅GEX₆
[FW₄]

wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and

wherein the VH comprises the amino acid sequence:

[FW₅]YYYMQ[FW₆]X₇IGSSGGVTNYADSVKG[FW₇]VGLGDAFDI[FW₈]

wherein [FW₅], [FW₆], [FW_{D}] and [FW₈] represent VH framework regions, and wherein

X₇ represents amino acid residues Tyrosine (Y), Isoleucine (I) or Valine (V).

The disclosure also provides, for use in the methods described herein, an isolated binding molecule (e.g., anti-HER3 antibody such as 2C2-YTE) or antigen binding fragment thereof which specifically binds to HER3 comprising an antibody VL, wherein the VL comprises a VL complementarity determining region-1 (VL-CDR1) amino acid sequence identical to, or identical except for four, three, two or one amino acid substitutions to: SEQ ID NO: 18, SEQ ID NO: 19, or SEQ ID NO: 20. Also, the disclosure provides, for use in the methods described herein, an isolated binding molecule (e.g., anti-HER3 antibody such as 2C2-YTE) or antigen binding fragment thereof which specifically binds to HER3 comprising an antibody VL, wherein the VL comprises a VL complementarity determining region-2 (VL-CDR2) amino acid sequence identical to, or identical except for four, three, two or one amino acid substitutions to SEQ ID NO: 21.

In addition, the disclosure provides, for use in the methods described herein, an isolated binding molecule (e.g., anti-HER3 antibody such as 2C2-YTE) or antigen binding fragment thereof which specifically binds to HER3 comprising an antibody VL, wherein the VL comprises a complementarity determining region-3 (VL-CDR3) amino acid sequence identical to, or identical except for four, three, two, or one amino acid substitutions to: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. Also, the disclosure provides, for use in the methods described herein, an isolated binding molecule (e.g., anti-HER3 antibody such as 2C2-YTE) or antigen binding fragment thereof which specifically binds to HER3 comprising an antibody VH, wherein the VH comprises a complementarity determining region-1 (VH-CDR1) amino acid sequence identical to, or identical except for four, three, two, or one amino acid substitutions to SEQ ID NO: 31.

Furthermore, the disclosure provides, for use in the methods described herein, an isolated binding molecule (e.g., anti-HER3 antibody such as 2C2-YTE) or antigen binding fragment thereof which specifically binds to HER3 comprising an antibody VH, wherein the VH comprises a complementarity determining region-2 (VH-CDR2) amino acid sequence identical to, or identical except for four, three, two, or one amino acid substitutions to: SEQ ID NO: 32, SEQ ID NO: 33, or SEQ ID NO: 34. Also provided, for use in the methods described herein, is an isolated binding molecule (e.g., anti-HER3 antibody such as 2C2-YTE) or antigen binding fragment thereof which specifically binds to HER3 comprising an antibody VH, wherein the VH comprises a complementarity determining region-3 (VH-CDR3) amino acid sequence identical to, or identical except for four, three, two, or one amino acid substitutions to SEQ ID NO: 35.

The disclosure provides, for use in the methods described herein, an isolated binding molecule (e.g., anti-HER3 antibody such as 2C2-YTE) or antigen binding fragment thereof which specifically binds to HER3 comprising an antibody VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 18, 21 and 22, SEQ ID NOs: 18, 21, and 26, SEQ ID NOs: 18, 21, and 27, SEQ ID NOs: 20, 21, and 22, SEQ ID NOs: 19, 21, and 22, SEQ ID NOs: 18, 21, and 25, SEQ ID NOs: 18, 21, and 28, SEQ ID NOs: 18, 21, and 29, SEQ ID NOs: 18, 21, and 30, SEQ ID NOs: 18, 21, and 23, SEQ ID NOs: 19, 21, and 23, SEQ ID NOs: 20, 21, and 23, SEQ ID NOs: 18, 21, and 24, or SEQ ID NOs: 18, 21, and 25, respectively. The disclosure also provides, for use in the methods described herein, an isolated binding molecule (e.g., anti-HER3 antibody such as 2C2-YTE) or antigen binding fragment thereof which specifically binds to HER3 comprising an antibody VH, wherein the VH comprises VH-CDR1, VH-CDR2, and VH-CDR3 amino acid sequences identical to, or identical except for four, three, two, or one amino acid substitutions in one or more of the VH-CDRS to: SEQ ID NOs: 31, 32 and 35, SEQ ID NOs: 31, 33, and 35, or SEQ ID NOs: 31, 34, and 35, respectively.

In addition, the disclosure provides, for use in the methods described herein, an isolated antibody (e.g., anti-HER3 antibody such as 2C2-YTE) or antigen-binding fragment thereof which specifically binds to HER3 comprising a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively. Also provided, for use in the methods described herein, is an isolated binding molecule (e.g., anti-HER3 antibody such as 2C2-YTE) or antigen binding fragment thereof which specifically binds to HER3 comprising an antibody VL and an antibody VH, wherein the VL comprises an amino acid sequence at least about 80% to about 90% identical or at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17. In a certain aspect, an isolated binding molecule (e.g., anti-HER3 antibody such as 2C2-YTE) or antigen binding fragment thereof which specifically binds to HER3 comprising an antibody VL and an antibody VH, wherein the VL comprises an amino acid sequence at least about 80% to about 90% identical or at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, wherein the VL CDRs are identical to those of the reference amino acid sequence. The disclosure also provides, for use in the methods described herein, an isolated binding molecule (e.g., anti-HER3 antibody such as 2C2-YTE) or antigen binding fragment thereof which specifically binds to HER3 comprising an antibody VL and an antibody VH, wherein the VH comprises an amino acid sequence at least about 80% to about 90% identical or at least about 90% to about 100% identical to a reference amino acid sequence selected from the group consisting of SEQ ID NO: 2, SEQ ID NO: 12 and SEQ ID NO: 13. In one aspect, an isolated binding molecule (e.g., anti-HER3 antibody such as 2C2-YTE) or antigen binding fragment thereof which specifically binds to HER3 comprising an antibody VL and an antibody VH, wherein the VH comprises an amino acid sequence at least about 80% to about 90% identical or at least about 90% to about 100% identical to a reference amino acid sequence selected from the group consisting of SEQ ID NO: 2, SEQ ID NO: 12 and SEQ ID NO: 13, wherein the VH CDRs are identical to those of the reference amino acid sequence. Furthermore, the disclosure provides, for use in the methods described herein, an isolated antibody (e.g., anti-HER3 antibody such as 2C2-YTE) or antigen binding fragment thereof which specifically binds to HER3, wherein the antibody or antigen binding fragment comprises a VL comprising a sequence at least about 80% to about 90% identical or at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the antibody or antigen binding fragment comprises a VH comprising a sequence at least about 80% to about 90% identical or at least about 90% to about 100% identical to a reference amino acid sequence selected from the group consisting of SEQ ID NO: 2, SEQ ID NO: 12 and SEQ ID NO: 13. an isolated antibody (e.g., anti-HER3 antibody such as 2C2-YTE) or antigen binding fragment thereof which specifically binds to HER3, wherein the antibody or antigen binding fragment comprises a VL comprising a sequence at least about 80% to about 90% identical or at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, wherein the VL CDRs are identical to those of the reference amino acid sequence, and wherein the antibody or antigen binding fragment comprises a VH comprising a sequence at least about 80% to about 90% identical or at least about 90% to about 100% identical to a reference amino acid sequence selected from the group consisting of SEQ ID NO: 2, SEQ ID NO: 12 and SEQ ID NO: 13, wherein the VH CDRs are identical to those of the reference amino acid sequence.

The disclosure also provides, for use in the methods described herein, an isolated antibody (e.g., anti-HER3 antibody such as 2C2-YTE) or antigen binding fragment thereof, which comprises a VL comprising SEQ ID NO: 49 and a VH comprising SEQ ID NO: 50. In addition, the disclosure provides an isolated antibody or antigen binding fragment thereof, which comprises a VL comprising SEQ ID NO: 3 and a VH comprising SEQ ID NO: 2. In a certain aspect, the anti-HER3 antibody or antigen-binding fragment comprises a VL comprising a sequence that is at least 80%, at least 85%, at least 90%, or at least 95% identical to SEQ ID NO: 3 and a VH comprising a sequence that is at least 80%, at least 85%, at least 90%, or at least 95% identical to SEQ ID NO: 2. In a certain aspect, the anti-HER3 antibody or antigen-binding fragment comprises a VL comprising a sequence that is at least 80%, at least 85%, at least 90%, or at least 95% identical SEQ ID NO: 3 and a VH comprising a sequence that is at least 80%, at least 85%, at least 90%, or at least 95% identical to SEQ ID NO: 2, wherein the VL CDRs are identical to those of SEQ ID NO: 3 and the VH CDRs are identical to those of SEQ ID NO: 2. Further, the disclosure provides, for use in the methods described herein, an isolated binding molecule (e.g., anti-HER3 antibody such as 2C2-YTE) or antigen-binding fragment thereof which specifically binds to an epitope within the extracellular domain of HER3, comprising an antibody VL of SEQ ID NO:3, an antibody VH of SEQ ID NO: 2, and an IgG1 constant region of SEQ ID 46. Also provided is an isolated binding molecule or antigen-binding fragment thereof which specifically binds to an epitope within the extracellular domain of HER3, consisting of an antibody VL of SEQ ID NO: 3, an antibody VH of SEQ ID NO: 2, and an IgG1 constant region of SEQ ID 46.

The disclosure also provides a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to an epitope within the extracellular domain of HER3, wherein the antibody specifically binds to the same HER3 epitope as an antibody or antigen-binding fragment thereof comprising the heavy chain variable region (VH) and light chain variable region (VL) of CL16 or 2C2.

The disclosure also provides a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3, and competitively inhibits HER3 binding by an antibody or antigen-binding fragment thereof comprising the VH and VL of CL16 or 2C2.

In certain aspects of methods provided herein, the VH and VL of CL16 comprise SEQ ID NOs: 2 and 1, respectively, and the VH and VL of 2C2 comprise SEQ ID NOs: 2 and 3, respectively.

In certain aspects of methods provided herein, the antibody or antigen binding fragment thereof is affinity matured.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VL, wherein the VL comprises the amino acid sequence:

[FW₁]X₁GSX₂SNIGLNYVS[FW₂]RNNQRPS[FW₃]AAWDDX₃X₄X₅GEX₆
[FW₄]

wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A).
   In certain aspects of methods provided herein, FW₁ comprises SEQ ID NO: 40 or 44, FW₂ comprises SEQ ID NO: 41, FW₃ comprises SEQ ID NO: 42, and FW₄ comprises SEQ ID NO: 43.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VH, wherein the VH comprises the amino acid sequence:

[FW₅]YYYMQ[FW₆]X₇IGSSGGVTNYADSVKG[FW₇]VGLGDAFDI[FW₈]

wherein [FW₅], [FW₆], [FW₇] and [FW₈] represent VH framework regions, and wherein

X₇ represents amino acid residues Tyrosine (Y), Isoleucine (I) or Valine (V).

In certain aspects of methods provided herein, FW₅ comprises SEQ ID NO: 36, FW₆ comprises SEQ ID NO: 37, FW₇ comprises SEQ ID NO: 38 and FW₈ comprises SEQ ID NO: 39.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VL and an antibody VH, wherein the VL comprises the amino acid sequence:

[FW₁]X₁GSX₂SNIGLNYVS[FW₂]RNNQRPS[FW₃]AAWDDX₃X₄X₅GEX₆
[FW₄]

wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and

wherein the VH comprises the amino acid sequence:

[FW₅]YYYMQ[FW₆]X₇IGSSGGVTNYADSVKG[FW₇]VGLGDAFDI[FW₈]

wherein [FW₅], [FW₆], [FW_{D}] and [FW₈] represent VH framework regions, and wherein

X₇ represents amino acid residues Tyrosine (Y), Isoleucine (I) or Valine (V).

In certain aspects of methods provided herein, FW₁ comprises SEQ ID NO: 40 or 44, FW₂ comprises SEQ ID NO: 41, FW₃ comprises SEQ ID NO: 42, FW₄ comprises SEQ ID NO: 43, FW₅ comprises SEQ ID NO: 36, FW₆ comprises SEQ ID NO: 37, FW₇ comprises SEQ ID NO: 38, and FW₈ comprises SEQ ID NO: 39.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VL, wherein the VL comprises a VL complementarity determining region-1 (VL-CDR1) amino acid sequence identical to, or identical except for four, three, two or one amino acid substitutions to: SEQ ID NO: 18, SEQ ID NO: 19, or SEQ ID NO: 20.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VL, wherein the VL comprises a VL complementarity determining region-2 (VL-CDR2) amino acid sequence identical to, or identical except for four, three, two or one amino acid substitutions to SEQ ID NO: 21.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VL, wherein the VL comprises a complementarity determining region-3 (VL-CDR3) amino acid sequence identical to, or identical except for four, three, two, or one amino acid substitutions to: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VH, wherein the VH comprises a complementarity determining region-1 (VH-CDR1) amino acid sequence identical to, or identical except for four, three, two, or one amino acid substitutions to SEQ ID NO: 31.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VH, wherein the VH comprises a complementarity determining region-2 (VH-CDR2) amino acid sequence identical to, or identical except for four, three, two, or one amino acid substitutions to: SEQ ID NO: 32, SEQ ID NO: 33, or SEQ ID NO: 34.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VH, wherein the VH comprises a complementarity determining region-3 (VH-CDR3) amino acid sequence identical to, or identical except for four, three, two, or one amino acid substitutions to SEQ ID NO: 35.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 18, 21 and 22, SEQ ID NOs: 18, 21, and 26, SEQ ID NOs: 18, 21, and 27, SEQ ID NOs: 20, 21, and 22, SEQ ID NOs: 19, 21, and 22, SEQ ID NOs: 18, 21, and 25, SEQ ID NOs: 18, 21, and 28, SEQ ID NOs: 18, 21, and 29, SEQ ID NOs: 18, 21, and 30, SEQ ID NOs: 18, 21, and 23, SEQ ID NOs: 19, 21, and 23, SEQ ID NOs: 20, 21, and 23, SEQ ID NOs: 18, 21, and 24, or SEQ ID NOs: 18, 21, and 25, respectively.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VH, wherein the VH comprises VH-CDR1, VH-CDR2, and VH-CDR3 amino acid sequences identical to, or identical except for four, three, two, or one amino acid substitutions in one or more of the VH-CDRS to: SEQ ID NOs: 31, 32 and 35, SEQ ID NOs: 31, 33, and 35, or SEQ ID NOs: 31, 34, and 35, respectively.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17. In a particular embodiment, the VL comprises VL CDRs identical to those of the reference amino acid sequence.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3 comprising an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13. In a particular embodiment, the VH comprises VH CDRs identical to those of the reference amino acid sequence.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3, wherein the antibody or antigen binding fragment comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the antibody or antigen binding fragment comprises a VH comprising a sequence at least about 90% to about 100% identical to a reference amino acid sequence selected from the group consisting of SEQ ID NO: 2, SEQ ID NO: 12 and SEQ ID NO: 13. In a particular embodiment, the VL comprises VL CDRs identical to those of the reference VL amino acid sequence, and the VH comprises VH CDRs identical to those of the reference VH amino acid sequence.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3, wherein the antibody or antigen binding fragment thereof comprises a VL comprising the VL consensus sequence provided in Table 3 and a VH comprising the VH consensus sequence provided in Table 3.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to HER3, wherein the antibody or antigen binding fragment thereof comprises a VL comprising SEQ ID NO: 3 and a VH comprising SEQ ID NO: 2.

In certain aspects of methods provided herein, the antibody or antigen binding fragment thereof comprises a heavy chain constant region or fragment thereof. In particular aspects, the heavy chain constant region or fragment thereof is an IgG constant region. In some aspects, the IgG constant region is selected from an IgG1 constant region, an IgG2 constant region, an IgG3 constant region and an IgG4 constant region. In specific aspects, the IgG constant region is an IgG1 constant region. In certain aspects, the antibody or antigen binding fragment thereof of the methods provided herein comprises a light chain constant region selected from the group consisting of a human kappa constant region and a human lambda constant region. In certain aspects, the IgG constant domain of the antibody or antigen binding fragment thereof of the methods provided herein comprises one or more amino acid substitutions relative to a wild-type IgG constant domain wherein the modified IgG has an increased half-life compared to the half-life of an IgG having the wild-type IgG constant domain. In particular aspects, the IgG constant domain comprises one or more amino acid substitutions of amino acid residues at positions 251-257, 285-290, 308-314, 385-389, and 428-436, wherein the numbering is according to the EU index as set forth in Kabat. In specific aspects, at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions,

wherein the numbering is according to the EU index as set forth in Kabat.

In specific aspects, the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
   wherein the numbering is according to the EU index as set forth in Kabat. In particular aspects, the amino acid at position 434 is substituted with an amino acid selected from the group consisting of Tryptophan (W), Methionine (M), Tyrosine (Y), and Serine (S), and wherein the numbering is according to the EU index as set forth in Kabat. In specific aspects, the amino acid at position 428 is substituted with an amino acid selected from the group consisting of Threonine (T), Leucine (L), Phenylalanine (F), and Serine (S), and wherein the numbering is according to the EU index as set forth in Kabat. In certain aspects, the amino acid at position 257 is substituted with Leucine (L), and the amino acid at Kabat position 434 is substituted with Tyrosine (Y), and wherein the numbering is according to the EU index as set forth in Kabat. In a particular aspect, the amino acid at Kabat position 428 is substituted with Leucine (L), and the amino acid at Kabat position 434 is substituted with Serine (S).

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to an epitope within the extracellular domain of HER3, comprising an antibody VL of SEQ ID NO:3, an antibody VH of SEQ ID NO: 2, and an IgG1 constant region of SEQ ID 46.

Also disclosed herein is a method of treating cancer in a subject, comprising (i) administering to the subject a therapeutically effective amount of a BRAF inhibitor; and (ii) administering to the subject a therapeutically effective amount of an antibody or antigen binding fragment thereof which specifically binds to an epitope within the extracellular domain of HER3, consisting of an antibody VL of SEQ ID NO: 3, an antibody VH of SEQ ID NO: 2, and an IgG1 constant region of SEQ ID 46.

In certain aspects of the methods described herein, the antibody is a fully human antibody, a humanized antibody, a chimeric antibody, a monoclonal antibody, a polyclonal antibody, a recombinant antibody, a multispecific antibody, or an antigen-binding fragment thereof.

In specific aspects of the methods described herein, the antigen-binding fragment is Fv, Fab, F(ab′)2, Fab′, dsFv, scFv, and sc(Fv)2.

In specific aspects of the methods described herein, the antibody or antigen binding fragment thereof is conjugated to at least one heterologous agent.

In particular aspects of the methods described herein, the antibody or antigen binding fragment can bind to human HER3, cynomolgus monkey HER3, and mouse HER3.

In certain aspects, the methods described herein further comprises administering to the subject a therapeutic effective amount of a MEK inhibitor. In particular aspects of the methods described herein, the MEK inhibitor is trametinib.

In specific aspects of the methods described herein for treating cancer, the cancer is selected from the group consisting of melanoma, thyroid cancer, colon/colorectal cancer, lung cancer (e.g., non-small cell lung cancer), pancreatic cancer, gastric cancer, breast cancer, and head and neck cancer (e.g., squamous cell carcinoma of the head and neck). In particular aspects of the methods described herein, the cancer comprises cells comprising a KRAS mutation. In certain aspects of the methods described herein, the KRAS mutation comprises a mutation of codon 12 of a human KRAS gene. In a specific aspect, the cancer is characterized by expression of HER3. In a specific aspect, the cancer is characterized by expression of Neuregulin 1/Heregulin 1. In a certain aspect, the cancer is characterized by a BRAF mutation (e.g., V600E or V600K).

In specific aspects of the methods described herein, the subject is human.

In certain aspects of the methods described herein, the BRAF inhibitor is vemurafenib. In certain aspects of the methods described herein, the BRAF inhibitor is dabrafenib.

In specific aspects of the methods described herein for treating cancer, the cancer comprises cells comprising a BRAF mutation. In certain aspects, the BRAF mutation is the BRAF V600E mutation. In certain aspects, the BRAF mutation is the BRAF V600K mutation. In a particular aspect, the BRAF inhibitor is an antibody or antigen binding fragment thereof.

In particular aspects of the methods described herein, the antibody or antigen binding fragment can specifically bind to an HER3 polypeptide or fragment thereof with an affinity characterized by a dissociation constant (K_{D}) of about 0.45 nM (+/−0.05 nM) or better as measured by a Biacore™ surface plasmon resonance assay wherein the antibody or antigen binding fragment thereof is bound to the Biacore™ chip.

In particular aspects of the methods described herein, the antibody or antigen binding fragment can specifically bind to an HER3 polypeptide or fragment thereof with an affinity characterized by a dissociation constant (K_{D}) of about 0.1 nM or better as measured by a Biacore™ surface plasmon resonance assay wherein the HER3 polypeptide or fragment thereof is bound to the Biacore™ chip.

In particular aspects of the methods described herein, wherein the antibody or antigen binding fragment can specifically bind to an HER3 polypeptide or fragment thereof with an affinity characterized by a Kₒₙ of between 1×10⁵ M⁻¹ s⁻¹ and 6×10⁵ M⁻¹ s⁻1 and/or a K_{off} of between 0.5×10⁻⁴ s⁻¹ and 2.0×10⁻⁴ s⁻¹ as measured by a Biacore™ surface plasmon resonance assay wherein the antibody or antigen binding or fragment thereof is bound to the Biacore™ chip.

### BRIEF DESCRIPTION OF THE DRAWINGS/FIGURES

FIG. 1A shows a multiple sequence alignment corresponding to the VL sequences of anti-HER3 monoclonal antibodies Clone 16 (CL16; original, parent clone), Clone 16 (GL; germlined clone), 5H6, 8A3, 4H6, 6E.3, 2B11, 2D1, 3A6 and 4C4. The location of CDR1, CDR2, and CDR3 is indicated. Amino acid residues which differ with respect to the CL16 (GL) antibody are highlighted.

FIG. 1B shows a multiple sequence alignment corresponding to the VH sequences of anti-HER3 monoclonal antibodies Clone 16 (CL16; parent clone), and clones 15D12.1 (also referred to as 15D12.I) and 15D12.2 (also referred to as 15D12.V). The locations of CDR1, CDR2, and CDR3 are indicated. Amino acid residues which differ with respect to the CL16 parent antibody are highlighted.

FIG. 1C shows a multiple sequence alignment corresponding to the VL sequences of anti-HER3 monoclonal antibodies CL16 (original, parent clone), CL16 (GL; germlined clone), 1A4, 2C2, 3E.1, 2F10, and 2B11. The location of CDR1, CDR2, and CDR3 is indicated. Amino acid residues which differ with respect to the CL16 (GL) antibody are highlighted.

FIG. 2 shows the change over time in mean serum concentration (in ng/mL) of 2C2-YTE after a first dose of 2C2-YTE for 5 mg/kg, 10 mg/kg and 20 mg/kg. Error bars indicate the standard deviation from the mean. The horizontal black line indicates a pharmacokinetic target serum concentration.

FIG. 3 shows the administration schema for a study to assess restoration of iodine incorporation in BRAF mutant (MUT), radioiodine-refractory (RAIR) thyroid cancer patients receiving 2C2-YTE and vemurafenib.

### DETAILED DESCRIPTION

The disclosure provides methods for administering anti-HER3 antibodies or antigen-binding fragments thereof, e.g., monoclonal antibodies capable of suppressing HER3 activity in both ligand-dependent and independent settings, to a patient in need thereof for preventing/protecting against or treating cancer (e.g., melanoma, head and neck cancer (e.g., squamous cell carcinoma of the head and neck), lung cancer (e.g., non-small cell lung cancer), colon/colorectal cancer, breast cancer, pancreatic cancer, thyroid cancer, or gastric cancer) or one or more symptoms thereof. The disclosure also provides methods of treating cancer (e.g., melanoma, head and neck cancer (e.g., squamous cell carcinoma of the head and neck), lung cancer (e.g., non-small cell lung cancer), colon/colorectal cancer, breast cancer, pancreatic cancer, thyroid cancer, or gastric cancer) in a human subject comprising administering an anti-HER3 antibody (e.g., 2C2-YTE) or an antigen-binding fragment thereof, for example, alone, or in combination with another agent, such as a chemotherapeutic agent (e.g., platinum), a MEK inhibitor (e.g., trametinib), a BRAF inhibitor (e.g., dabrafenib or vemurafenib), an EGFR inhibitor (e.g., an anti-EGFR antibody such as cetuximab or reversible or irreversible small molecule EGFR inhibitor such as erlotinib or AZD9291) or a HER2 inhibitor (e.g., an anti-HER2 antibody such as trastuzumab), or with the combination of a BRAF inhibitor and a MEK inhibitor.

In one aspect, the disclosure provides methods of treating cancer (e.g., melanoma, head and neck cancer (e.g., squamous cell carcinoma of the head and neck), lung cancer (e.g., non-small cell lung cancer), colon/colorectal cancer, breast cancer, pancreatic cancer, thyroid cancer, or gastric cancer) in a human subject comprising administering to a human subject an amount of an anti-HER3 antibody (e.g., 2C2-YTE) or an antigen-binding fragment thereof sufficient to achieve a serum concentration (or serum PK level) at or above a target serum concentration (e.g., Cₘ or trough concentration) throughout the dosing interval in the subject, for monotherapy or combination therapy, for example, in combination with a MEK inhibitor (e.g., trametinib), a BRAF inhibitor (e.g., dabrafenib or vemurafenib), an EGFR inhibitor (e.g., cetuximab or erlotinib) or a HER2 inhibitor (e.g., trastuzumab) or with the combination of a BRAF inhibitor and a MEK inhibitor.

Provided herein are methods of treating cancer (e.g., head and neck cancer, colon cancer, non-small cell lung cancer, melanoma, breast cancer, or gastric cancer) in a subject comprising administering to the subject an anti-HER3 antibody (e.g., 2C2-YTE) or an antigen-binding fragment thereof at a dose of 15 mg/kg or 20 mg/kg, in combination with a BRAF inhibitor (e.g., dabrafenib or vemurafenib), an EGFR inhibitor (e.g., cetuximab or erlotinib) or a HER2 inhibitor (e.g., trastuzumab). In one embodiment, the dose of the anti-HER3 antibody or antigen-binding fragment thereof is 15 mg/kg. In particular aspects, provided herein are methods for treating head and neck cancer or KRAS mutated, EGFR expressing colon cancer in a human subject comprising administering to the subject an anti-HER3 antibody (e.g., 2C2-YTE) or an antigen-binding fragment thereof at a dose of 15 mg/kg or 20 mg/kg every 21 days, in combination with an EGFR inhibitor such as cetuximab. In one embodiment, the dose of the anti-HER3 antibody or antigen-binding fragment thereof is 15 mg/kg. In particular aspects, provided herein are methods for treating non-small cell lung cancer in a human subject comprising administering to the subject an anti-HER3 antibody (e.g., 2C2-YTE) or an antigen-binding fragment thereof at a dose of 15 mg/kg or 20 mg/kg every 21 days, in combination with an EGFR inhibitor such as erlotinib. In one embodiment, the dose of the anti-HER3 antibody or antigen-binding fragment thereof is 15 mg/kg. In particular aspects, provided herein are methods for treating melanoma, such as BRAF mutated melanoma, in a human subject comprising administering to the subject an anti-HER3 antibody (e.g., 2C2-YTE) or an antigen-binding fragment thereof at a dose of 15 mg/kg or 20 mg/kg every 21 days, in combination with a BRAF inhibitor such as vemurafenib. In one embodiment, the dose of the anti-HER3 antibody or antigen-binding fragment thereof is 15 mg/kg. In particular aspects, provided herein are methods for treating HER2 positive breast cancer or gastric cancer in a human subject comprising administering to the subject an anti-HER3 antibody (e.g., 2C2-YTE) or an antigen-binding fragment thereof at a dose of 15 mg/kg or 20 mg/kg every 21 days, in combination with a HER2 inhibitor such as trastuzumab. In one embodiment, the dose of the anti-HER3 antibody or antigen-binding fragment thereof is 15 mg/kg.

In certain aspects, provided herein are methods of treating cancer (e.g., melanoma, such as BRAF mutated melanoma) in a subject comprising administering to the subject an anti-HER3 antibody (e.g., 2C2-YTE antibody) at a dose of 15 mg/kg or 20 mg/kg, in combination with a BRAF inhibitor (e.g., dabrafenib or vemurafenib) and a MEK inhibitor (e.g., selumetinib or trametinib). In one embodiment, the dose of the anti-HER3 antibody or antigen-binding fragment thereof is 15 mg/kg.

In particular aspects, the methods provided herein for treating cancer associated with a BRAF mutation, such as BRAF V600E or V600K mutation. BRAF or B-Raf (v-raf murine sarcoma viral oncogene homolog B1) is an intracellular serine/threonine kinase component of the mitogen-activated protein kinase (MAPK) pathway. A non-limiting example of an amino acid sequence of human BRAF is provided with GenBank Accession No. NP_004324. Within the cluster of Raf serine-threonine kinases there are 3 isoforms: A-Raf, B-Raf, and C-Raf. Certain mutations in BRAF have been associated with tumorogenesis. For example, the valine-to-glutamate (V600E) point mutation in BRAF is a constitutively active mutation which activates MEK without first needing upstream Ras phosphorylation and leads to the constitutive activation of EGFR signaling through the oncogenic Ras/Raf/Mek/Erk pathway. Another non-limiting example of a BRAF mutation is the BRAF V600K mutation. BRAF mutation occurs at relatively high frequency in certain cancers, such as colorectal cancer. Due to its ability to constitutively activate the ERK pathway, mutant BRAF has been shown to confer tumor resistance to RTK therapies, for example EGFR mAbs such as Cetuximab and Panitumumab.

In certain aspects, the methods provided herein for treating cancer relate to treating cancer with a KRAS mutation, which lead to the constitutive activation of EGFR signaling through the oncogenic Ras/Raf/Mek/Erk pathway. Kras mutation is among the most-frequently occurring mutation events in many solid tumors, for example, colorectal (CRC), pancreatic and lung cancers (LC). Due to its ability to constitutively activate the ERK pathway, mutant KRAS has been shown to confer tumor resistance to RTK therapies, for example EGFR mAbs such as Cetuximab and Panitumumab.

In specific aspects, provided herein are methods of monitoring treatment, or response to treatment, of a human subject diagnosed with cancer, wherein the treatment comprises administering an anti-HER3 antibody (e.g., 2C2-YTE) or antigen-binding fragment thereof which specifically binds to an epitope within the extracellular domain of HER3, comprising measuring in a sample from the human subject the expression level of one or more pharmacodynamic markers, such as soluble HER3, TWEAK, TWEAK receptor, Neurexophilin-1, IL-18 binding protein, ANGL4, IL-18 receptor 1, or Kallikrein 7.

In particular aspects, provided herein are binding molecules (e.g., anti-HER3 antibody such as 2C2-YTE) and antigen-binding fragments thereof that bind to HER3. In some aspects, such molecules are antibodies and antigen-binding fragments thereof that specifically bind to HER3. Related polynucleotides, compositions comprising the anti-HER3 antibodies or antigen-binding fragments thereof, and methods of making the anti-HER3 antibodies and antigen-binding fragments are also provided.

### I. Terminology

As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used herein.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects, which can be had by reference to the specification as a whole. Accordingly, the terms defined immediately below are more fully defined by reference to the specification in its entirety.

It is understood that wherever aspects are described herein with the language “comprising,” otherwise analogous aspects described in terms of “consisting of” and/or “consisting essentially of” are also provided.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The terms “HER3” and “HER3 receptor” are used interchangeably herein, and refer to the ErbB3 protein (also referred to as HER3, ErbB3 receptor in the literature) as described in U.S. Pat. No. 5,480,968 and in Plowman et al. (1990) Proc. Natl. Acad. Sci. USA 87, 4905-4909; see also, Kani et al. (2005) Biochemistry 44, 15842-15857, and Cho & Leahy (2002) Science 297, 1330-1333. The full-length, mature HER3 protein sequence (without leader sequence) corresponds to the sequence shown in FIG. 4 and SEQ ID NO: 4 of U.S. Pat. No. 5,480,968 minus the 19 amino acid leader sequence that is cleaved from the mature protein.

The terms “HER” and “HER receptor” are used interchangeably herein, and refer to one or more, or all, members of the epidermal growth factor receptor (EGFR) EGFR/HER subfamily of receptor protein tyrosine kinases (RTK), consisting of EGFR (HER1/Erbb1), HER2/Erbb2, HER3/Erbb3 and HER4/Erbb4. In a particular aspect, the terms “HER” and “HER receptor” are used interchangeably herein, and refer to EGFR, HER2, HER3, or HER4.

The terms “HER2,” and “HER2 receptor” are used interchangeably herein, and refer to the ErbB2 protein (also referred to as HER2, ErbB2 receptor, neu, or p185 in the literature), for example, as described in Yamamoto et al., 1986, Nature, 319:230-234; Bargmann et al., 1986, Nature, 319: 226-230; and Di Fiore et al., 1991, Methods Enzymol, 198:272-277. A non-limiting example of an amino acid sequence of human HER2 is provided with GenBank Accession No. NP_004439 or NP_001005862. A non-limiting example of a nucleotide sequence encoding human HER2 is provided with GenBank Accession No. NM_001005862.

The terms “EGFR,” “HER1,” and “HER1 receptor” are used interchangeably herein, and refer to the EGFR protein (also referred to as HER1, or ErbB1 receptor in the literature), for example, as described in Lin et al., Science, 1984, 224:843-848. A non-limiting example of an amino acid sequence of human EGFR is provided with GenBank Accession No. NP_005219.2. A non-limiting example of a nucleotide sequence encoding human EGFR is provided with GenBank Accession No. NM_005228.3.

The terms “inhibition” and “suppression” are used interchangeably herein and refer to any statistically significant decrease in biological activity, including full blocking of the activity. For example, “inhibition” can refer to a decrease of about 10%, 20%, 30%, 40%, 50%, 60%, 70%, 80%, 90% or 100% in biological activity. In certain embodiments, the term “about” means within plus or minus 10% of a given value or range. In certain embodiments, where an integer is required, the term “about” means within plus or minus 10% of a given value or range, rounded either up or down to the nearest integer. Accordingly, when the terms “inhibition” or “suppression” are applied to describe, e.g., an effect on ligand-mediated HER3 phosphorylation, the term refers to the ability of an antibody or antigen binding fragment thereof to statistically significantly decrease the phosphorylation of HER3 induced by an EGF-like ligand, relative to the phosphorylation in an untreated (control) cell. The cell which expresses HER3 can be a naturally occurring cell or cell line (e.g., a cancer cell) or can be recombinantly produced by introducing a nucleic acid encoding HER3 into a host cell. In one aspect, the anti-HER3 binding molecule, e.g., an antibody or antigen binding fragment thereof inhibits ligand mediated phosphorylation of HER3 by at least 10%, or at least 20%, or at least 30%, or at least 40%, or at least 50%, or at least 60%, or at least 70%, or at least 80%, or at least 905, or about 100%, as determined, for example, by Western blotting followed by probing with an anti-phosphotyrosine antibody or by ELISA.

The term “growth suppression” of a cell expressing HER3, as used herein, refer to the ability of anti-HER3 binding molecule, e.g., an antibody or antigen-binding fragment thereof to statistically significantly decrease proliferation of a cell expressing HER3 relative to the proliferation in the absence of the anti-HER3 binding molecule, e.g., an antibody or antigen-binding fragment thereof. In one aspect, the proliferation of a cell expressing HER3 (e.g., a cancer cell) can be decreased by at least 10%, or at least 20%, or at least 30%, or at least 40%, or at least 50%, or at least 60%, or at least 70%, or at least 80%, or at least 90%, or about 100% when cells are contacted with an anti-HER3 binding molecule, e.g., an antibody or antigen-binding fragment thereof of described herein, relative to the proliferation measured in the absence of the anti-HER3 binding molecule, e.g., an antibody or antigen-binding fragment thereof (control conditions). Cellular proliferation can be assayed using art recognized techniques with measure rate of cell division, the fraction of cells within a cell population undergoing cell division, and/or rate of cell loss from a cell population due to terminal differentiation or cell death (e.g., thymidine incorporation).

The terms “antibody” or “immunoglobulin,” as used interchangeably herein, include whole antibodies and any antigen binding fragment or single chains thereof.

A typical antibody comprises at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed Complementarity Determining Regions (CDR), interspersed with regions that are more conserved, termed framework regions (FW). Each VH and VL is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Exemplary antibodies of the present disclosure include the Clone 16 (CL16) anti-HER3 antibodies (original and germlined), affinity optimized clones including for example, the anti-HER3 2C2 antibody, and serum half-life-optimized anti-HER3 antibodies including for example the anti-HER3 2C2-YTE antibody.

The term “germlining” means that amino acids at specific positions in an antibody are mutated back to those in the germ line. E.g., the CL16 “germlined” antibody is generated from the original CL16 antibody by introducing three point mutations, Y2S, E3V and M20I, into FW1 of the VL regions.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

A “blocking” antibody or an “antagonist” antibody is one which inhibits or reduces biological activity of the antigen it binds, such as HER3. In a certain aspect blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “HER3 antibody” or “an antibody that binds to HER3” or “anti-HER3” refers to an antibody that is capable of binding HER3 with sufficient affinity such that the antibody is useful as a therapeutic agent or diagnostic reagent in targeting HER3. The extent of binding of an anti-HER3 antibody to an unrelated, non-HER3 protein is less than about 10% of the binding of the antibody to HER3 as measured, e.g., by a radioimmunoassay (RIA), BIACORE™ (using recombinant HER3 as the analyte and antibody as the ligand, or vice versa), or other binding assays known in the art. In certain aspects, an antibody that binds to HER3 has a dissociation constant (K_{D}) of ≦1 μM, ≦100 nM, ≦10 nM, ≦1 nM, ≦0.1 nM, ≦10 pM, ≦1 pM, or ≦0.1 pM.

The terms “antigen binding fragment” refers to a portion of an intact antibody and refers to the antigenic determining variable regions of an intact antibody. It is known in the art that the antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FW) connected by three complementarity determining regions (CDRs) also known as hypervariable regions. The CDRs in each chain are held together in close proximity by the FW regions and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

TABLE 1

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H353 | H26-H35B | H26-H32_34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs. See e.g., Lefranc, M. P. et al., Dev. Comp. Immunol. 27: 55-77(2003), which is herein incorporated by reference. The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used throughout the specification the VH CDRs sequences described correspond to the classical Kabat numbering locations, namely Kabat VH-CDR1 is at positions 31-35, VH-CDR2 is a positions 50-65, and VH-CDR3 is at positions 95-102. VL-CDR2 and VL-CDR3 also correspond to classical Kabat numbering locations, namely positions 50-56 and 89-97, respectively. As used herein, the terms “VL-CDR1” or “light chain CDR1” correspond to sequences located at Kabat positions 23-34 in the VL (in contrast, the classical VL-CDR1 location according to the Kabat numbering schema corresponds to positions 24-34).

As used herein the Fc region includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as set forth in Kabat (Kabat et al., Sequences of Proteins of Immunological Interest, 5th Ed. Public Health Service, National Institutes of Health, Bethesda, Md. (1991)). Fc may refer to this region in isolation, or this region in the context of an antibody, antibody fragment, or Fc fusion protein. Polymorphisms have been observed at a number of different Fc positions, including but not limited to positions 270, 272, 312, 315, 356, and 358 as numbered by the EU index, and thus slight differences between the presented sequence and sequences in the prior art may exist.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc) with the desired specificity, affinity, and capability while the constant regions are homologous to the sequences in antibodies derived from another (usually human) to avoid eliciting an immune response in that species.

The terms “YTE” or “YTE mutant” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al. (1991) Sequences of Proteins of Immunological Interest, U.S. Public Health Service, National Institutes of Health, Washington, D.C.), introduced into the heavy chain of an IgG1. See U.S. Pat. No. 7,658,921, which is incorporated by reference herein. The YTE mutant has been shown to increase the serum half-life of antibodies approximately four-times as compared to wild-type versions of the same antibody (Dall'Acqua et al., J. Biol. Chem. 281:23514-24 (2006)). See also U.S. Pat. No. 7,083,784, which is hereby incorporated by reference in its entirety.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes described herein.

“Potency” is normally expressed as an IC₅₀ value, in nM unless otherwise stated. IC₅₀ is the median inhibitory concentration of an antibody molecule. In functional assays, IC₅₀ is the concentration that reduces a biological response by 50% of its maximum. In ligand-binding studies, IC₅₀ is the concentration that reduces receptor binding by 50% of maximal specific binding level. IC₅₀ can be calculated by any number of means known in the art. Improvement in potency can be determined by measuring, e.g., against the parent CL16 (Clone 16) monoclonal antibody.

The fold improvement in potency for the antibodies or polypeptides described herein as compared to a Clone 16 antibody can be at least about 2-fold, at least about 4-fold, at least about 6-fold, at least about 8-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, or at least about 180-fold or more.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are absolutely required for such killing. Lysis of the target cell is extracellular, requires direct cell-to-cell contact, and does not involve complement. It is contemplated that, in addition to antibodies, other proteins comprising Fc regions, specifically Fc fusion proteins, having the capacity to bind specifically to an antigen-bearing target cell will be able to effect cell-mediated cytotoxicity. For simplicity, the cell-mediated cytotoxicity resulting from the activity of an Fc fusion protein is also referred to herein as ADCC activity.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

An “effective amount” of an antibody as disclosed herein is an amount sufficient to carry out a specifically stated purpose. An “effective amount” can be determined empirically and in a routine manner, in relation to the stated purpose.

The term “therapeutically effective amount” refers to an amount of an antibody or other drug effective to “treat” a disease or disorder in a subject or mammal.

The word “label” when used herein refers to a detectable compound or composition which is conjugated directly or indirectly to the antibody so as to generate a “labeled” antibody. The label can be detectable by itself (e.g., radioisotope labels or fluorescent labels) or, in the case of an enzymatic label, can catalyze chemical alteration of a substrate compound or composition which is detectable.

Terms such as “treating” or “treatment” or “to treat” or “alleviating” or “to alleviate” refer to therapeutic measures that cure, slow down, lessen symptoms of, and/or halt progression of a diagnosed pathologic condition or disorder and slow the development of a targeted pathologic condition or disorder. Thus, those in need of treatment include those already with the disorder; those prone to have the disorder; and those in whom the disorder is to be prevented. In certain aspects, a subject is successfully “treated” for cancer according to the methods described herein if the patient shows, e.g., total, partial, or transient remission of a certain type of cancer.

For the purposes of the present disclosure it should be understood that phrases like “Compound X for use in treating . . . ,” “Use of compound X in the manufacture of a composition for treating . . . ,” or “Method of treating of . . . by administering compound X to . . . ” all circumscribe that a compound X (such as in the present case anti-HER3 antibodies or antigen-binding fragments thereof) is used for a specific treatment (such as in the present case the treatment of cancer).

The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include brain cancer, CNS cancer, squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes et al. (2006) Br. J. Cancer 94:1057-1065), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer and cancers of mucinous origins, such as, mucinous ovarian cancer, cholangiocarcinoma (liver) and renal papillary carcinoma.

As used herein, the term “carcinomas” refers to cancers of epithelial cells, which are cells that cover the surface of the body, produce hormones, and make up glands. Examples of carcinomas are cancers of the skin, lung, colon, stomach, breast, prostate and thyroid gland.

The term “KRAS mutation,” as used herein, refers to mutations found in certain cancers in a human homolog of the v-Ki-ras2 Kirsten rat sarcoma viral oncogene. Non-limiting examples of human KRAS gene mRNA sequences include Genbank Accession Nos. NM004985 and NM033360. It has been reported that KRAS mutations are found in 73% of pancreatic tumors, 35% of colorectal tumors, 16% of ovarian tumors and 17% of lung tumors. KRAS mutation generally occur in codons 12 or 143 of the human KRAS gene.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA.

The term “vector” means a construct, which is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides described herein are based upon antibodies, in certain aspects, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain aspects, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain aspects, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative aspects, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain aspects, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain aspects, the default parameters of the alignment software are used.

In certain aspects, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain aspects, conservative substitutions in the sequences of the polypeptides and antibodies described herein do not abrogate the binding of the polypeptide or antibody containing the amino acid sequence, to the antigen(s), i.e., the HER3 to which the polypeptide or antibody binds. Methods of identifying nucleotide and amino acid conservative substitutions which do not eliminate antigen binding are well-known in the art (see, e.g., Brummell et al., Biochem. 32: 1180-1187 (1993); Kobayashi et al., Protein Eng. 12(10):879-884 (1999); and Burks et al., Proc. Natl. Acad. Sci. USA 94:412-417 (1997)).

The term “consensus sequence,” as used herein with respect to light chain (VL) and heavy chain (VH) variable regions, refers to a composite or genericized VL or VH sequence defined based on information as to which amino acid residues within the VL or VH chain are amenable to modification without detriment to antigen binding. Thus, in a “consensus sequence” for a VL or VH chain, certain amino acid positions are occupied by one of multiple possible amino acid residues at that position. For example, if an arginine (R) or a serine (S) occur at a particular position, then that particular position within the consensus sequence can be either arginine or serine (R or S). Consensus sequences for VH and VL chain can be defined, for example, by in vitro affinity maturation (e.g., randomizing every amino acid position in a certain CDR using degenerate coding primers), by scanning mutagenesis (e.g., alanine scanning mutagenesis) of amino acid residues within the antibody CDRs, or any other methods known in the art, followed by evaluation of the binding of the mutants to the antigen to determine whether the mutated amino acid position affects antigen binding. In some aspects, mutations are introduced in the CDR regions. In other aspects, mutations are introduced in framework regions. In some other aspects, mutations are introduced in CDR and framework regions.

As used herein, the terms “Cmin,” “Cₘᵢₙ,” “trough level” and “trough concentration” refer to the lowest levels of an agent, for example an antibody (e.g., anti-HER3 antibody such as 2C2 or 2C2-YTE antibody), in a sample (e.g., a serum or plasma sample) from a subject over a period of time. In certain embodiments, the period of time is the entire period of time between the administration of one dose of an agent, for example an antibody (e.g., anti-HER3 antibody such as 2C2 or 2C2-YTE antibody), and another dose of the agent, for example an antibody (e.g., anti-HER3 antibody such as 2C2 or 2C2-YTE antibody). In some embodiments, the period of time is approximately 1 week, approximately 2 weeks, approximately 3 weeks, or approximately 4 weeks after the administration of one dose of an agent, for example an antibody (e.g., anti-HER3 antibody such as 2C2 or 2C2-YTE antibody) and before the administration of another dose of the agent, for example an antibody (e.g., anti-HER3 antibody such as 2C2 or 2C2-YTE antibody). In certain embodiments, the term “approximately” means within plus or minus 10% of a given value or range. In certain embodiments, where an integer is required, the term “approximately” means within plus or minus 10% of a given value or range, rounded either up or down to the nearest integer.

A “high” level refers to a level (e.g., of expression, for example, mRNA or protein expression, such as cell surface protein expression) that is greater than normal, for example, greater than a level in a “reference sample,” or a level that is greater than a particular standard. The reference sample may be normal/healthy cells, or may be all cancer cells, or may be a particular subset of cancer cells. For example, a reference sample may be a subset of cancer cells that do not express a neuregulin (e.g., colorectal cancer cells, for example the colorectal cancer cells used in the examples herein). A “high” level may also refer to a level that is higher than a predetermined amount or measure, such as a predetermined cutoff amount. A group of samples may be divided around a particularly determined value (for example, a median, a mean, or a quartile), or a particularly determined threshold (such as, for example, a line on a graph), such that two subgroups exist, one that is considered to have a “high” level (e.g., of expression, for example, mRNA or protein expression, such as cell surface protein expression) and one that is considered to have a “low” level (or, that does not have a “high” level). As used herein, a “high level of expression” is used interchangeably with “overexpression,” and “exhibits a high level of expression” is used interchangeably with “overexpresses.” In a certain embodiment, a “high” level refers to a level of expression (e.g., mRNA expression or protein expression, such as cell-surface expression) that is 2-fold, 3-fold, 4-fold, 5-fold, 8-fold or 10-fold the median level observed across cancer cell types. mRNA, protein and cell-surface protein expression can be determined using techniques well known in the art, e.g. RT-PCR, ELISA, and flow cytometry techniques, respectively.

### II. Anti-HER3-Binding Molecules

Provided herein, for use in methods of treating cancer, are HER3 binding molecules, e.g., antibodies and antigen-binding fragments thereof that specifically bind HER3 (e.g., CL16, 2C2 or 2C2-YTE antibodies). The full-length amino acid (aa) and nucleotide (nt) sequences for HER3 are known in the art (see, e.g., UniProt Acc. No. P2186 for human HER3, or UniProt Acc. No. 088458 for mouse HER3). In some aspects, the anti-HER3 binding molecules are human antibodies. In certain aspects, the HER3 binding molecules are antibodies or antigen-binding fragments thereof. In some aspects, HER3 binding molecules, e.g., antibodies or antigen-binding fragments thereof comprise a Fab, a Fab′, a F(ab′)₂, a Fd, a single chain Fv or scFv, a disulfide linked Fv, a V-NAR domain, an IgNar, an intrabody, an IgGΔCH2, a minibody, a F(ab′)₃, a tetrabody, a triabody, a diabody, a single-domain antibody, DVD-Ig, Fcab, mAb², a (scFv)₂, or a scFv-Fc. In some aspects, the antibody is of the IgG1 subtype and comprises the triple mutant YTE, as disclosed supra in the Definitions section.

In certain aspects, anti-HER3 antibodies or antigen-binding fragments thereof described herein are modified compared to the parent Clone 16 (CL16) antibody. The modifications can include mutations in the CDR regions and/or in the FW regions as compared to CL16. In certain aspects, an anti-HER3 antibody described herein comprises modifications to CDR1 and/or CDR3 of the light chain of CL16, including, but not limited to:

1) a light chain CDR1 comprising the consensus sequence X₁GSX₂SNIGLNYVS, wherein X₁ is selected from R or S, and X₂ is selected from S or L; and

2) a light chain CDR3 comprising the consensus sequence AAWDDX₃X₄X₅GEX₆, wherein X₃ is selected from S or G, X₄ is selected from L or P, X₅ is selected from R, I, P or S, and X₆ is selected from V or A.

In certain aspects, an anti-HER3 antibody or antigen-binding fragment thereof described herein comprises modifications to CDR2 of the heavy chain of CL16, including, but not limited to a heavy chain CDR1 comprising the consensus sequence X₇IGSSGGVTNYADSVKG, wherein X₇ is selected from Y, I or V.

In one aspect, an anti-HER3 antibody or antigen binding fragment thereof comprises a VL region comprising the consensus amino acid sequence:

[FW₁]X₁GSX₂SNIGLNYVS[FW₂]RNNQRPS[FW₃]AAWDDX₃X₄X₅GEX₆
[FW₄]

wherein [FW₁], [FW₂], [FW₃] and [FW₄] represent the amino acid residues of VL framework region 1 (SEQ ID NO: 40 or 44), VL framework region 2 (SEQ ID NO: 41), VL framework region 3 (SEQ ID NO: 42) and VL framework region 4 (SEQ ID NO: 43), and wherein X₁ represents amino acid residues arginine (R) or serine (S), X₂ represents amino acid residues serine (S) or leucine (L), X₃ represents amino acid residues serine (S) or glutamic acid (E), X₄ represents amino acid residues leucine (L) or proline (P), X₅ represents amino acid residues arginine (R), isoleucine (I), proline (P) or serine (S), and X₆ represents amino acid residues valine (V) or arginine (R).

In one aspect, an anti-HER3 antibody or antigen binding fragment thereof comprises a VH region comprises the consensus amino acid sequence:

[FW₅]YYYMQ[FW₆]X₇IGSSGGVTNYADSVKG[FW₇]VGLGDAFDI[FW₈]

wherein [FW₅], [FW₆], [FW₇] and [FW₈] represent the amino acid residues of VH framework region 1 (SEQ ID NO: 36), VH framework region 2 (SEQ ID NO: 37), VH framework region 3 (SEQ ID NO: 38) and VH framework region 4 (SEQ ID NO: 39), and wherein X₇ represents amino acid residues tyrosine (Y), isoleucine (I) or valine (V).

In one aspect, an anti-HER3 antibody or antigen binding fragment thereof comprises a VL region comprising the consensus amino acid sequence:

[FW₁]X₁GSX₂SNIGLNYVS[FW₂]RNNQRPS[FW₃]AAWDDX₃X₄X₅GEX₆
[FW₄]

wherein [FW₁], [FW₂], [FW₃] and [FW₄] represent the amino acid residues of VL framework region 1 (SEQ ID NO: 40 or 44), VL framework region 2 (SEQ ID NO: 41), VL framework region 3 (SEQ ID NO: 42) and VL framework region 4 (SEQ ID NO: 43), and wherein X₁ represents amino acid residues arginine (R) or serine (S), X₂ represents amino acid residues serine (S) or leucine (L), X₃ represents amino acid residues serine (S) or glutamic acid (E), X₄ represents amino acid residues leucine (L) or proline (P), X₅ represents amino acid residues arginine (R), isoleucine (I), proline (P) or serine (S), and X₆ represents amino acid residues valine (V) or arginine (R); and wherein said anti-HER3 antibody or antigen binding fragment thereof further comprises a VH region which comprises the consensus amino acid sequence:

[FW₅]YYYMQ[FW₆]X₇IGSSGGVTNYADSVKG[FW₇]VGLGDAFDI[FW₈]

wherein [FW₅], [FW₆], [FW₇] and [FW₈] represent the amino acid residues of VH framework region 1 (SEQ ID NO: 36), VH framework region 2 (SEQ ID NO: 37), VH framework region 3 (SEQ ID NO: 38) and VH framework region 4 (SEQ ID NO: 39), and wherein X₇ represents amino acid residues tyrosine (Y), isoleucine (I) or valine (V).

In some aspects, an anti-HER3 antibody or antigen-binding fragment thereof described herein comprises a VL-CDR1 consisting of sequence selected from the group consisting of SEQ ID NOs: 18, 19 and 20. In some aspects, an anti-HER3 antibody or antigen-binding fragment thereof described herein comprises a VL-CDR1 comprising a sequence selected from the group consisting of SEQ ID NOs: 18, 19 and 20. In some aspects, an anti-HER3 antibody or antigen-binding fragment thereof described herein comprises a VL-CDR2 consisting of SEQ ID NO: 21. In some aspects, an anti-HER3 antibody or antigen-binding fragment thereof described herein comprises a VL-CDR2 comprising SEQ ID NO: 21. In some aspects, an anti-HER3 antibody or antigen-binding fragment thereof described herein comprises a VL-CDR3 consisting of a sequence selected from the group consisting of SEQ ID NOs: 22, 23, 24, 25, 26, 27, 28, 29, and 30. In some aspects, an anti-HER3 antibody or antigen-binding fragment thereof described herein comprises a VL-CDR3 comprising a sequence selected from the group consisting of SEQ ID NOs: 22, 23, 24, 25, 26, 27, 28, 29, and 30.

In some aspects, an anti-HER3 antibody or antigen-binding fragment thereof described herein comprises a VH-CDR1 consisting of SEQ ID NO: 31. In some aspects, an anti-HER3 antibody or antigen-binding fragment thereof described herein comprises a VH-CDR1 comprising SEQ ID NO: 31. In some aspects, an anti-HER3 antibody or antigen-binding fragment thereof described herein comprises a VH-CDR2 consisting of a sequence selected from the group consisting of SEQ ID NOs: 32, 33 and 34. In some aspects, an anti-HER3 antibody or antigen-binding fragment thereof described herein comprises a VH-CDR2 comprising a sequence selected from the group consisting of SEQ ID NOs: 32, 33 and 34. In some aspects, an anti-HER3 antibody or antigen-binding fragment thereof described herein comprises a VH-CDR3 consisting of SEQ ID NO: 35. In some aspects, an anti-HER3 antibody or antigen-binding fragment thereof described herein comprises a VH-CDR3 comprising SEQ ID NO: 35.

In some aspects, an anti-HER3 antibody or antigen-binding fragment thereof described herein comprises a VL-CDR1 consisting of a sequence selected from the group consisting of SEQ ID NOs: 18, 19 and 20, except for one, two, three or four amino acid substitutions. In some aspects, an anti-HER3 antibody or antigen-binding fragment thereof described herein comprises a VL-CDR1 comprising a sequence selected from the group consisting of SEQ ID NOs: 18, 19 and 20, except for one, two, three or four amino acid substitutions. In some aspects, an anti-HER3 antibody or antigen-binding fragment thereof described herein comprises a VL-CDR2 consisting of SEQ ID NO: 21, except for one, two, three or four amino acid substitutions. In some aspects, an anti-HER3 antibody or antigen-binding fragment thereof described herein comprises a VL-CDR2 comprising SEQ ID NO: 21, except for one, two, three or four amino acid substitutions. In some aspects, an anti-HER3 antibody or antigen-binding fragment thereof described herein comprises a VL-CDR3 consisting of a sequence selected from the group consisting of SEQ ID NOs: 22, 23, 24, 25, 26, 27, 28, 29, and 30, except for one, two, three or four amino acid substitutions. In some aspects, an anti-HER3 antibody or antigen-binding fragment thereof described herein comprises a VL-CDR3 comprising a sequence selected from the group consisting of SEQ ID NOs: 22, 23, 24, 25, 26, 27, 28, 29, and 30, except for one, two, three or four amino acid substitutions.

In some aspects, an anti-HER3 antibody or antigen-binding fragment thereof described herein comprises a VH-CDR1 consisting of SEQ ID NO: 31, except for one, two, three or four amino acid substitutions. In some aspects, an anti-HER3 antibody or antigen-binding fragment thereof described herein comprises a VH-CDR1 comprising SEQ ID NO: 31, except for one, two, three or four amino acid substitutions. In some aspects, an anti-HER3 antibody or antigen-binding fragment thereof described herein comprises a VH-CDR2 consisting of a sequence selected from the group consisting of SEQ ID NOs: 32, 33 and 34, except for one, two, three or four amino acid substitutions. In some aspects, an anti-HER3 antibody or antigen-binding fragment thereof described herein comprises a VH-CDR2 comprising a sequence selected from the group consisting of SEQ ID NOs: 32, 33 and 34, except for one, two, three or four amino acid substitutions. In some aspects, an anti-HER3 antibody or antigen-binding fragment thereof described herein comprises a VH-CDR3 consisting of SEQ ID NO: 35, except for one, two, three or four amino acid substitutions. In some aspects, an anti-HER3 antibody or antigen-binding fragment thereof described herein comprises a VH-CDR3 comprising SEQ ID NO: 35, except for one, two, three or four amino acid substitutions.

In some aspects, an anti-HER3 antibody or antigen-binding fragment thereof described herein comprises a VL-CDR1 consisting of a sequence selected from the group consisting of SEQ ID NOs: 18, 19 and 20; a VL-CDR2 consisting of SEQ ID NO: 21; and a VL-CDR3 consisting of a sequence selected from the group consisting of SEQ ID NOs: 22, 23, 24, 25, 26, 27, 28, 29, and 30. In some aspects, an anti-HER3 antibody or antigen-binding fragment thereof described herein comprises a VL-CDR1 comprising a sequence selected from the group consisting of SEQ ID NOs: 18, 19 and 20; a VL-CDR2 comprising SEQ ID NO: 21; and a VL-CDR3 comprising a sequence selected from the group consisting of SEQ ID NOs: 22, 23, 24, 25, 26, 27, 28, 29, and 30.

In some aspects, an anti-HER3 antibody or antigen-binding fragment thereof described herein comprises a VH-CDR1 consisting of SEQ ID NO: 31; a VH-CDR2 consisting of a sequence selected from the group consisting of SEQ ID NOs: 32, 33 and 34; and a VH-CDR3 consisting of SEQ ID NO: 35. In some aspects, an anti-HER3 antibody or antigen-binding fragment thereof described herein comprises a VH-CDR1 comprising SEQ ID NO: 31; a VH-CDR2 comprising a sequence selected from the group consisting of SEQ ID NOs: 32, 33 and 34; a VH-CDR3 comprising SEQ ID NO: 35.

In some aspects, an anti-HER3 antibody or antigen-binding fragment thereof described herein comprises a VL-CDR1 consisting of a sequence selected from the group consisting of SEQ ID NOs: 18, 19 and 20, except for one, two, three or four amino acid substitutions; a VL-CDR2 consisting of SEQ ID NO: 21, except for one, two, three or four amino acid substitutions; and a VL-CDR3 consisting of a sequence selected from the group consisting of SEQ ID NOs: 22, 23, 24, 25, 26, 27, 28, 29, and 30, except for one, two, three or four amino acid substitutions. In some aspects, an anti-HER3 antibody or antigen-binding fragment thereof described herein comprises a VL-CDR1 comprising a sequence selected from the group consisting of SEQ ID NOs: 18, 19 and 20, except for one, two, three or four amino acid substitutions; a VL-CDR2 comprising SEQ ID NO: 21, except for one, two, three or four amino acid substitutions; and a VL-CDR3 comprising a sequence selected from the group consisting of SEQ ID NOs: 22, 23, 24, 25, 26, 27, 28, 29, and 30, except for one, two, three or four amino acid substitutions.

In some aspects, an anti-HER3 antibody or antigen-binding fragment thereof described herein comprises a VH-CDR1 consisting of SEQ ID NO: 31, except for one, two, three or four amino acid substitutions; a VH-CDR2 consisting of a sequence selected from the group consisting of SEQ ID NOs: 32, 33 and 34, except for one, two, three or four amino acid substitutions; and a VH-CDR3 consisting of SEQ ID NO: 35, except for one, two, three or four amino acid substitutions. In some aspects, an anti-HER3 antibody or antigen-binding fragment thereof antibody described herein comprises a VH-CDR1 comprising SEQ ID NO: 31, except for one, two, three or four amino acid substitutions; a VH-CDR2 comprising a sequence selected from the group consisting of SEQ ID NOs: 32, 33 and 34, except for one, two, three or four amino acid substitutions; and VH-CDR3 comprising SEQ ID NO: 35, except for one, two, three or four amino acid substitutions.

In certain aspects, an anti-HER3 antibody or antigen-binding fragment thereof described herein comprises modifications to CDR1, CDR2, and/or CDR3 of the heavy and/or light chain, and further comprises modifications to FW1, FW2, FW3, and/or FW4 of the heavy and/or light chain. In some aspects, FW₁ comprises SEQ ID NO: 40 or 44, FW₂ comprises SEQ ID NO: 41, FW₃ comprises SEQ ID NO: 42, FW₄ comprises SEQ ID NO: 43, FW₅ comprises SEQ ID NO: 36, FW₆ comprises SEQ ID NO: 37, FW₇ comprises SEQ ID NO: 38, and FW₈ comprises SEQ ID NO: 39.

In some aspects, FW₁ comprises SEQ ID NO: 40 or 44, except for one, two, three or four amino acid substitutions; FW₂ comprises SEQ ID NO: 41, except for one, two, three or four amino acid substitutions; FW₃ comprises SEQ ID NO: 42, except for one, two, three or four amino acid substitutions; FW₄ comprises SEQ ID NO: 43, except for one, two, three or four amino acid substitutions; FW₅ comprises SEQ ID NO: 36, except for one, two, three or four amino acid substitutions; FW₆ comprises SEQ ID NO: 37, except for one, two, three or four amino acid substitutions; FW₇ comprises SEQ ID NO: 38, except for one, two, three or four amino acid substitutions; and FW₈ comprises SEQ ID NO: 39, except for one, two, three or four amino acid substitutions.

In certain aspects, an anti-HER3 antibody or antigen-binding fragment thereof described herein comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

Heavy and light chain variable domains of the anti-HER3 antibody or antigen-binding fragment thereof described herein include the sequences listed in TABLE 2.

TABLE 2

| SEQ | | |
|---|---|---|
| ID | | |
| NO. | Description | Sequence |
| 1 | CL16VL | QSVLTQPPSASGTPGQRVTISCSGSSSNIGLNYVSWYQQLPGTAPKLLISRNNQRP |
| | (Germlined) | SGVPDRFSGSKSGTSASLAISGLRSEDEADYYCAAWDDSLSGEVFGGGTKLTVL |
| 17 | CL16VL | QYELTQPPSASGTPGQRVTMSCSGSSSNIGLNYVSWYQQLPGTAPKLLISRNNQR |
| | (original) | PSGVPDRFSGSKSGTSASLAISGLRSEDEADYYCAAWDDSLSGEVFGGGTKLTVL |
| 2 | CL16 VH | EVQLLESGGGLVQPGGSLRLSCAASGFTFSYYYMQWVRQAPGKGLEWVSYIGS |
| | | SGGVTNYADSVKGRFTISRDNSKNTLYLQMNSLRAEDTAVYYCARVGLGDAFD |
| | | IWGQGTMVTVSS |
| 4 | 5H6 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDGLPGEVFGGGTKLTVL |
| 5 | 8A3 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSLIGEVFGGGTKLTVL |
| 6 | 4H6 VL | QSVLTQPPSASGTPGQRVTISCRGSSSNIGLNYVSWYQQLPGTAPKLLISRNNQRP |
| | | SGVPDRFSGSKSGTSASLAISGLRSEDEADYYCAAWDDSLSGEVFGGGTKLTVL |
| 7 | 6E.3 VL | QSVLTQPPSASGTPGQRVTISCSGSLSNIGLNYVSWYQQLPGTAPKLLISRNNQRP |
| | | SGVPDRFSGSKSGTSASLAISGLRSEDEADYYCAAWDDSLSGEVFGGGTKLTVL |
| 8 | 2B11 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSLPGEVFGGGTKLTVL |
| 9 | 2D1 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSLSGEAFGGGTKLTVL |
| 10 | 3A6 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSPSGEVFGGGTKLTVL |
| 11 | 4C4 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSLRGEVFGGGTKLTVL |
| 12 | 15D12 .1 | EVQLLESGGGLVQPGGSLRLSCAASGFTFSYYYMQWVRQAPGKGLEWVSIIGSS |
| | (15D12.1) | GGVTNYADSVKGRFTISRDNSKNTLYLQMNSLRAEDTAVYYCARVGLGDAFDI |
| | VH | WGQGTMVTVSS |
| 13 | 15D12.2 | EVQLLESGGGLVQPGGSLRLSCAASGFTFSYYYMQWVRQAPGKGLEWVSVIGS |
| | (15D12.V) | SGGVTNYADSVKGRFTISRDNSKNTLYLQMNSLRAEDTAVYYCARVGLGDAFD |
| | VH | IWGQGTMVTVSS |
| 14 | 1A4 VL | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSPPGEAFGGGTKLTVL |
| 3 | 2C2 VL | QSVLTQPPSASGTPGQRVTISCSGSLSNIGLNYVSWYQQLPGTAPKLLISRNNQRP |
| | | SGVPDRFSGSKSGTSASLAISGLRSEDEADYYCAAWDDSPPGEAFGGGTKLTVL |
| 15 | 3E.1 VL | QSVLTQPPSASGTPGQRVTISCRGSSSNIGLNYVSWYQQLPGTAPKLLISRNNQRP |
| | | SGVPDRFSGSKSGTSASLAISGLRSEDEADYYCAAWDDSPPGEAFGGGTKLTVL |
| 16 | 2F10 | QSVLTQPPSASGTPGQRVTISCSGSSSNIGLNYVSWYQQLPGTAPKLLISRNNQRP |
| | | SGVPDRFSGSKSGTSASLAISGLRSEDEADYYCAAWDDSPSGEAFGGGTKLTVL |

In certain aspects, an anti-HER3 antibody or antigen-binding fragment thereof described herein comprises an antibody VL and an antibody VH, wherein the VL comprises an amino acid sequence at least about 80%, about 85%, about 90%, about 95%, about 96%, about 97%, about 98%, about 99%, or about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17. In a specific embodiment, the VL comprises VL CDRs identical to those of the VL reference amino acid sequence.

In other aspects, an anti-HER3 antibody or antigen-binding fragment thereof described herein comprises an antibody VL and an antibody VH, wherein the VH comprises an amino acid sequence at least about 80%, about 85%, about 90%, about 95%, about 96%, about 97%, about 98%, about 99%, or about 100% identical to a reference amino acid sequence selected from the group consisting of SEQ ID NO: 2, SEQ ID NO: 12 and SEQ ID NO: 13. In a specific embodiment, the VH comprises VH CDRs identical to those of the VL reference amino acid sequence.

In other aspects, an anti-HER3 antibody or antigen-binding fragment thereof described herein comprises a VL comprising a sequence at least about 80%, about 85%, about 90%, about 95%, about 96%, about 97%, about 98%, about 99%, or about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and further comprises a VH comprising a sequence at least about 80%, about 85%, about 90%, about 95%, about 96%, about 97%, about 98%, about 99%, or about 100% identical to a reference amino acid sequence selected from the group consisting of SEQ ID NO: 2, SEQ ID NO: 12 and SEQ ID NO: 13. In a specific embodiment, the VL comprises VL CDRs identical to those of the VL reference amino acid sequence, and the VH comprises VH CDRs identical to those of the VH reference amino acid sequence. In a certain aspect, an anti-HER3 antibody or antigen-binding fragment provided herein comprises a VL comprising a sequence that is at least 80%, at least 85%, at least 90%, or at least 95% identical to SEQ ID NO: 3 and a VH comprising a sequence that is at least 80%, at least 85%, at least 90%, or at least 95% identical to SEQ ID NO: 2. In a certain aspect, an anti-HER3 antibody or antigen-binding fragment provided herein comprises a VL comprising a sequence that is at least 80%, at least 85%, at least 90%, or at least 95% identical SEQ ID NO: 3 and a VH comprising a sequence that is at least 80%, at least 85%, at least 90%, or at least 95% identical to SEQ ID NO: 2, wherein the VL CDRs are identical to those of SEQ ID NO: 3 and the VH CDRs are identical to those of SEQ ID NO: 2.

In some aspects, an anti-HER3 antibody or antigen-binding fragment thereof comprises a VH of TABLE 2 and a VL of TABLE 2. Antibodies are designated throughout the specification according to their VL chains. The heavy chains of the specific antibodies disclosed in the present specification correspond to the CL16 original heavy chain (SEQ ID NO: 2). Thus, the “CL16 antibody” is an IgG1 comprising two original CL16 light chains (SEQ ID NO: 17) and two CL16 original heavy chains (SEQ ID NO: 2), whereas the “2C2 antibody” is an IgG1 comprising two 2C2 light chains (2C2 VL (SEQ ID NO: 3) and two CL16 original heavy chains (SEQ ID NO: 2).

In some aspects, the anti-HER3 antibody or antigen-binding fragment thereof comprises a heavy chain constant region or fragment thereof. In some specific aspects, the heavy chain constant region is an IgG constant region. The IgG constant region can comprise a light chain constant region selected from the group consisting of a kappa constant region and a lambda constant region.

In certain aspects, an anti-HER3 antibody or antigen-binding fragment thereof described herein binds HER3 with substantially the same or better affinity as the CL16 antibody, comprising the CL16 original heavy chain (SEQ ID NO: 2) and the original CL16 light chain (SEQ ID NO: 17). In certain aspects, an anti-HER3 antibody or antigen-binding fragment thereof described herein binds HER3 with substantially the same or better affinity as the 2C2 antibody, comprising the 2C2 light chain (2C2 VL (SEQ ID NO: 3) and the CL16 original heavy chain (SEQ ID NO: 2).

In one aspect provided herein, an anti-HER3 antibody or antigen-binding fragment thereof specifically binds HER3 and antigenic fragments thereof with a dissociation constant of k_{d} (k_{off}/kₒₙ) of less than 10⁻⁶ M, or of less than 10⁻⁷ M, or of less than 10⁻⁸ M, or of less than 10⁻⁹ M, or of less than 10⁻¹⁰ M, or of less than 10⁻¹¹ M, or of less than 10⁻¹² M, or of less than 10⁻¹³ M. In a particular aspect provided herein, an anti-HER3 antibody or antigen-binding fragment thereof specifically binds HER3 and antigenic fragments thereof with a dissociation constant between 2×10⁻¹⁰ M and 6×10⁻¹⁰ M.

In another aspect, an anti-HER3 antibody or antigen-binding fragment thereof described herein binds to HER3 and/or antigenic fragments thereof with a K_{off} of less than 1×10⁻³ s⁻¹, or less than 2×10⁻³ s⁻¹. In other aspects, an anti-HER3 antibody or antigen-binding fragment thereof binds to HER3 and antigenic fragments thereof with a K_{off} of less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹. In a particular aspect, an anti-HER3 antibody or antigen-binding fragment thereof described herein binds to HER3 and/or antigenic fragments thereof with a K_{off} of between 0.5×10⁻⁴ s⁻¹ and 2.0×10⁻⁴ s⁻¹.

In another aspect, an anti-HER3 antibody or antigen-binding fragment thereof described herein binds to HER3 and/or antigenic fragments thereof with an association rate constant or kₒₙ rate of at least 10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 108 M−1 s−1, or at least 109 M−1 s−1. In another aspect, an anti-HER3 antibody or antigen-binding fragment thereof described herein binds to HER3 and/or antigenic fragments thereof with an association rate constant or kon rate of between 1×105 M−1 s−1 and 6×105 M−1 s−1.

The VH and VL sequences disclosed in TABLE 1 can be “mixed and matched” to create other anti-HER3 binding molecules described herein. In certain aspects, the VH sequences of 15D12.I and 15D12.V are mixed and matched. Additionally or alternatively, the VL sequences of 5H6, 8A3, 4H6, 6E.3, 2B11, 2D1, 3A6, 4C4, 1A4, 2C2, 3E.1 can be mixed and matched.

In certain aspects, an anti-HER3 antibody or antigen-binding fragment thereof described herein comprises mutations that improve the binding to human FcRn and improve the half-life of the anti-HER3 antibody or antigen-binding fragment thereof. In some aspects, such mutations are a methionine (M) to tyrosine (Y) mutation in position 252, a serine (S) to threonine (T) mutation in position 254, and a threonine (T) to glutamic acid (E) mutation in position 256, numbered according to the EU index as in Kabat (Kabat, et al. (1991) Sequences of Proteins of Immunological Interest, U.S. Public Health Service, National Institutes of Health, Washington, D.C.), introduced into the constant domain of an IgG1. See U.S. Pat. No. 7,658,921, which is incorporated by reference herein. This type of mutant IgG, referred to as a “YTE mutant” has been shown display approximately four-times increased half-life as compared to wild-type versions of the same antibody (Dall'Acqua et al., J. Biol. Chem. 281:23514-24 (2006)). In some aspects, an anti-HER3 antibody or antigen-binding fragment thereof comprising an IgG constant domain comprises one or more amino acid substitutions of amino acid residues at positions 251-257, 285-290, 308-314, 385-389, and 428-436, numbered according to the EU index as in Kabat, wherein such mutations increase the serum half-life of the anti-HER3 antibody or antigen-binding fragment thereof.

In some aspects, a YTE mutant further comprises a substitution at position 434 of the IgG constant domain, numbered according to the EU index as in Kabat, with an amino acid selected from the group consisting of tryptophan (W), methionine (M), tyrosine (Y), and serine (S). In other aspects, a YTE mutant further comprises a substitution at position 434 of the IgG constant domain, numbered according to the EU index as in Kabat, with an amino acid selected from the group consisting of tryptophan (W), methionine (M), tyrosine (Y), and serine (S), and substitution at position 428 of the IgG constant domain, numbered according to the EU index as in Kabat, with an amino acid selected from the group consisting of threonine (T), leucine (L), phenylalanine (F), and serine (S).

In yet other aspect, a YTE mutant further comprises a substitution at position 434 of the IgG constant domain, numbered according to the EU index as in Kabat, with tyrosine (Y), and a substitution at position 257 of the IgG constant domain, numbered according to the EU index as in Kabat, with leucine (L). In some aspects, a YTE mutant further comprises a substitution at position 434 of the IgG constant domain, numbered according to the EU index as in Kabat, with serine (S), and a substitution at position 428 of the IgG constant domain, numbered according to the EU index as in Kabat, with leucine (L).

In a specific aspect, an anti-HER3 antibody or antigen-binding fragment thereof comprises a 2C2 light chain variable region (2C2 VL; SEQ ID NO: 3), an original CL16 heavy chain variable region (SEQ ID NO: 2), and an IgG1 constant domain comprising a methionine (M) to tyrosine (Y) mutation in position 252, a serine (S) to threonine (T) mutation in position 254, and a threonine (T) to glutamic acid (E) mutation in position 256 of the IgG1 constant domain, numbered according to the EU index as in Kabat.

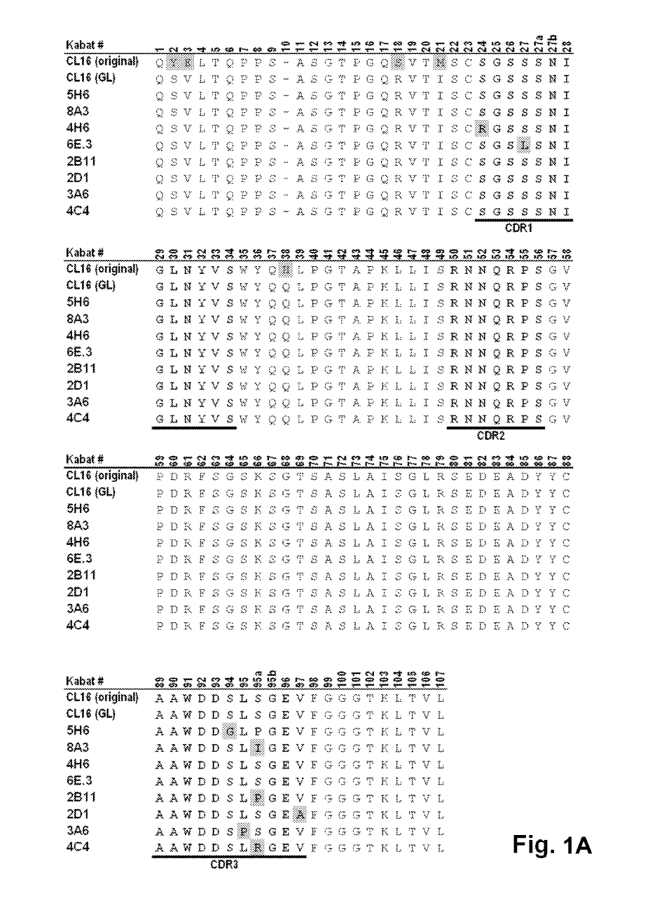

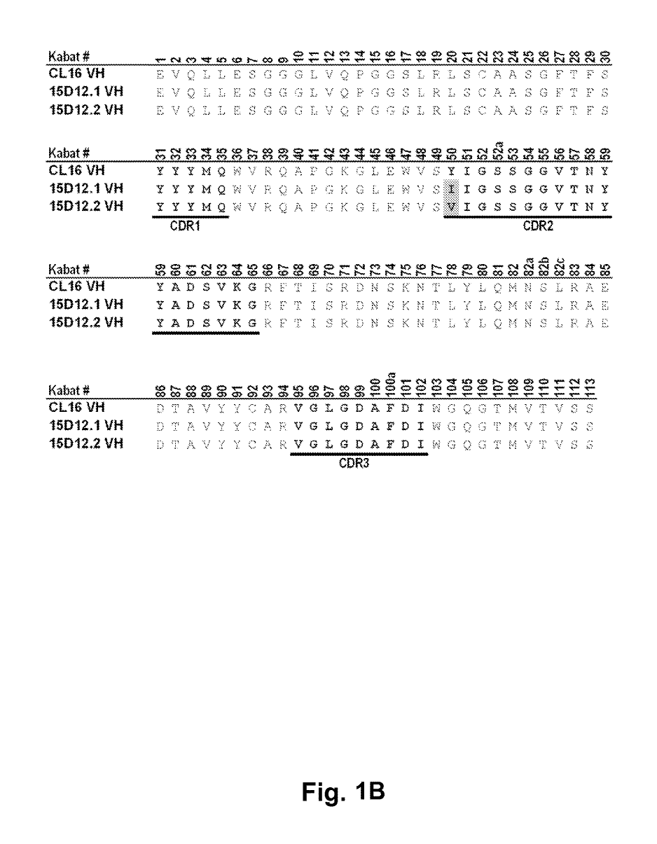

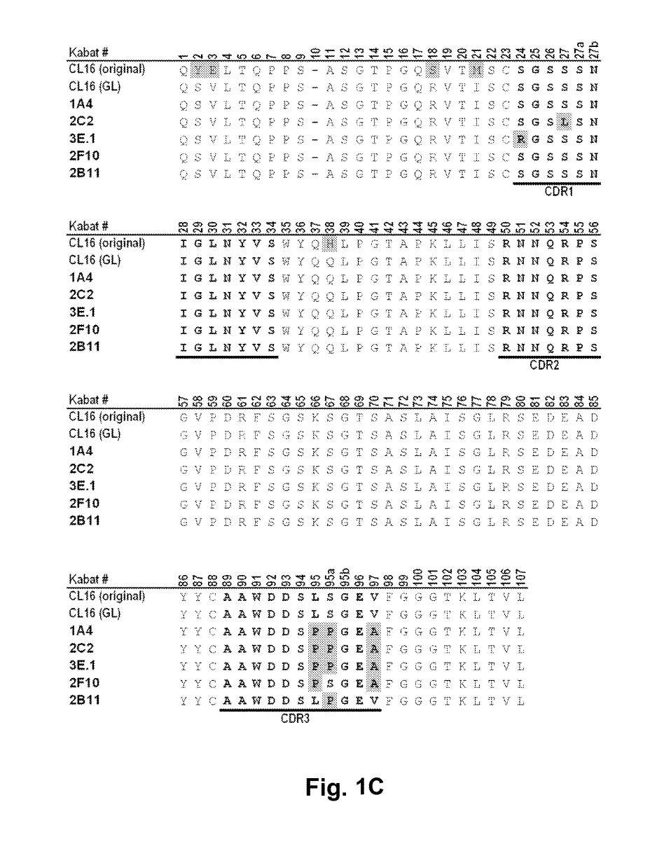

In a specific aspect, an anti-HER3 antibody or antigen-binding fragment thereof comprises a light chain variable region and a heavy chain variable region as presented in FIGS. 1A-1C or Table 3. Alignment of the amino acid sequences of the light and heavy chain variable regions for certain single mutations is shown in FIG. 1A and FIG. 1B, respectively. TABLE 3 provides the SEQ ID NOs for each clone. An alignment of the light chain variable regions for each of the combination clones is provided in FIG. 1C.

TABLE 3

| SEQ | | SEQ | |
|---|---|---|---|
| ID | DESCRIPTION | ID | DESCRIPTION |
| 17 | Clone 16 V_{L} aa | 21 | Clone 4H6 V_{L} CDR2 aa |
| 1 | Clone 16-germlined V_{L} aa | 22 | Clone 4H6 V_{L} CDR3 aa |
| 2 | Clone 16 V_{H} aa | 7 | Clone 6E.3 V_{L} aa |
| 18 | Clone 16 V_{L} CDR1 aa | 19 | Clone 6E.3 V_{L} CDR1 aa |
| 21 | Clone 16 V_{L} CDR2 aa | 21 | Clone 6E.3 V_{L} CDR2 aa |
| 22 | Clone 16 V_{L} CDR3 aa | 22 | Clone 6E.3 V_{L} CDR3 aa |
| 31 | Clone 16 V_{H} CDR1 aa | 9 | Clone 2D1 V_{L} aa |
| 32 | Clone 16 V_{H} CDR2 aa | 18 | Clone 2D1 V_{L} CDR1 aa |
| 35 | Clone 16 V_{H} CDR3 aa | 21 | Clone 2D1 V_{L} CDR2 aa |
| 8 | Clone 2B11 V_{L} aa | 28 | Clone 2D1 V_{L} CDR3 aa |
| 18 | Clone 2B11 V_{L} CDR1 aa | 10 | Clone 3A6 V_{L} aa |
| 21 | Clone 2B11 V_{L} CDR2 aa | 18 | Clone 3A6 V_{L} CDR1 aa |
| 25 | Clone 2B11 V_{L} CDR3 aa | 21 | Clone 3A6 V_{L} CDR2 aa |
| 14 | Clone 1A4 V_{L} aa | 29 | Clone 3A6 V_{L} CDR3 aa |
| 18 | Clone 1A4 V_{L} CDR1 aa | 11 | Clone 4C4 V_{L} aa |
| 21 | Clone 1A4 V_{L} CDR2 aa | 18 | Clone 4C4 V_{L} CDR1 aa |
| 22 | Clone 1A4 V_{L} CDR3 aa | 21 | Clone 4C4 V_{L} CDR2 aa |
| 3 | Clone 2C2 V_{L} aa | 30 | Clone 4C4 V_{L} CDR3 aa |
| 19 | Clone 2C2 V_{L} CDR1 aa | 12 | Clone 15D12.1 V_{H} aa |
| 21 | Clone 2C2 V_{L} CDR2 aa | 31 | Clone 15D12.1 V_{H} CDR1 aa |
| 23 | Clone 2C2 V_{L} CDR3 aa | 33 | Clone 15D12.1 V_{H} CDR2 aa |
| 16 | Clone 2F10 V_{L} aa | 35 | Clone 15D12.1 V_{H} CDR3 aa |
| 18 | Clone 2F10 V_{L} CDR1 aa | 13 | Clone 15D12.2 V_{H} aa |
| 21 | Clone 2F10 V_{L} CDR2 aa | 31 | Clone 15D12.2 V_{H} CDR1 aa |
| 24 | Clone 2F10 V_{L} CDR3 aa | 34 | Clone 15D12.2 V_{H} CDR2 aa |
| 15 | Clone 3E.1 V_{L} aa | 35 | Clone 15D12.2 V_{H} CDR3 aa |
| 20 | Clone 3E.1 V_{L} CDR1 aa | 36 | V_{H} FW1 aa |
| 21 | Clone 3E.1 V_{L} CDR2 aa | 37 | V_{H} FW2 aa |
| 23 | Clone 3E.1 V_{L} CDR3 aa | 38 | V_{H} FW3 aa |
| 4 | Clone 5H6 V_{L} aa | 39 | V_{H} FW4 aa |
| 18 | Clone 5H6 V_{L} CDR1 aa | 40 | V_{L} FW1 germlined aa |
| 21 | Clone 5H6 V_{L} CDR2 aa | 41 | V_{L} FW2 aa |
| 26 | Clone 5H6 V_{L} CDR3 aa | 42 | V_{L} FW3 aa |
| 5 | Clone 8A3 V_{L} aa | 43 | V_{L} FW4 aa |
| 18 | Clone 8A3 V_{L} CDR1 aa | 44 | V_{L} FW1 original aa |
| 21 | Clone 8A3 V_{L} CDR2 aa | 45 | IgG1 constant region* |
| 27 | Clone 8A3 V_{L} CDR3 aa | 46 | IgG1 constant region* - YTE |
| 6 | Clone 4H6 V_{L} aa | 47 | Clone 16 V_{L} nt |
| 20 | Clone 4H6 V_{L} CDR1 aa | 48 | Clone 16 V_{H} nt |

*allotype differences are provided
V_{L} aa consensus:
[FW₁]X₁GSX₂SNIGLNYVS[FW₂]RNNQRPS[FW₃]AAWDDX₃X₄X₅GEX₆[FW₄] wherein [FW₁], [FW₂], [FW₃] and [FW₄] represent VL framework regions, wherein
(a) X₁ represents amino acid residues Arginine (R) or Serine (S),
(b) X₂ represents amino acid residues Serine (S) or Leucine (L),
(c) X₃ re presents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A).
V_{H} aa consensus:
[FW₅]YYYMQ[FW₆]X₇IGSSGGVTNYADSVKG[FW₇]VGLGDAFDI[FW₈] wherein [FW₅], [FW₆], [FW₇] and [FW₈] represent VH framework regions, wherein X₇ represents amino acid residues Tyrosine (Y), Isoleucine (I) or Valine (V)

In a specific aspect, an anti-HER3 antibody or antigen-binding fragment thereof comprises a light chain variable region and a heavy chain variable region as described in PCT International Publication No. WO 2013/078191 A1, which is hereby incorporated by reference in its entirety.

In certain aspects, an anti-HER3 antibody or antigen-binding fragment thereof described herein comprise at least one IgG constant domain amino acid substitution selected from the group consisting of:
(a) substitution of the amino acid at position 252 with tyrosine (Y), phenylalanine (F), tryptophan (W), or threonine (T),
(b) substitution of the amino acid at position 254 with threonine (T),
(c) substitution of the amino acid at position 256 with serine (S), arginine (R), glutamine (Q), glutamic acid (E), aspartic acid (D), or threonine (T),
(d) substitution of the amino acid at position 257 with leucine (L),
(e) substitution of the amino acid at position 309 with proline (P),
(f) substitution of the amino acid at position 311 with serine (S),
(g) substitution of the amino acid at position 428 with threonine (T), leucine (L), phenylalanine (F), or serine (S),
(h) substitution of the amino acid at position 433 with arginine (R), serine (S), isoleucine (I), proline (P), or glutamine (Q),
(i) substitution of the amino acid at position 434 with tryptophan (W), methionine (M), serine (S), histidine (H), phenylalanine (F), or tyrosine, and
(j) a combination of two or more of said substitutions,
wherein the positions are numbered according to the EU index as in Kabat, and wherein the modified IgG has an increased serum half-life compared to the serum half-life of an IgG having the wild-type IgG constant domain.

In other aspects, the VH and/or VL amino acid sequences can be at least 85%, 90%, 95%, 96%, 97%, 98% or 99% identical to the sequences set forth above, and comprise 1, 2, 3, 4, 5 or more conservative substitutions. In certain aspects, the VH and/or VL amino acid sequences can be at least 85%, 90%, 95%, 96%, 97%, 98% or 99% identical to the sequences set forth above, and comprise 1, 2, 3, 4, 5 or more conservative substitutions which are not within the VH CDRs or VL CDRs. In other aspects, the VH and/or VL amino acid sequences can be at least 80%, 85%, 90%, or 95% identical to the sequences set forth above, and comprise 1, 2, 3, 4, 5 or more conservative substitutions. In certain aspects, the VH and/or VL amino acid sequences can be at least 80%, 85%, 90%, or 95% identical to the sequences set forth above, and comprise 1, 2, 3, 4, 5 or more conservative substitutions which are not within the VH CDRs or VL CDRs. A HER3 antibody having VH and VL regions having high (i.e., 80% or greater) similarity to the VH regions of SEQ ID NOs: 2, 12 or 13 and/or VL regions of SEQ ID NOs: 1, 3, 4, 5, 6, 7, 8, 9, 10, 11, 14, 15, 16, or 17, respectively, can be obtained by mutagenesis (e.g., site-directed or PCR-mediated mutagenesis) of nucleic acid molecules encoding SEQ ID NOs: 1-17, followed by testing of the encoded altered antibody for retained function using the functional assays described herein.

The affinity or avidity of an antibody for an antigen can be determined experimentally using any suitable method well known in the art, e.g., flow cytometry, enzyme-linked immunosorbent assay (ELISA), or radioimmunoassay (RIA), or kinetics (e.g., BIACORE™ analysis). Direct binding assays as well as competitive binding assay formats can be readily employed. (See, for example, Berzofsky et al., “Antibody-Antigen Interactions,” In Fundamental Immunology, Paul, W. E., Ed., Raven Press: New York, N.Y. (1984); Kuby, Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., K_{D} or Kd, Kₒₙ, K_{off}) are made with standardized solutions of antibody and antigen, and a standardized buffer, as known in the art and such as the buffer described herein.

It also known in the art that affinities measured using BIACORE™ analysis can vary depending on which one of the reactants is bound to the chip. In this respect, affinity can be measured using a format in which the targeting antibody (e.g., the 2C2 monoclonal antibody) is immobilized onto the chip (referred to as an “IgG down” format) or using a format in which the target protein (e.g., HER3) is immobilized onto the chip (referred to as, e.g., a “HER3 down” format).

### III. Binding Molecules that Bind to the Same Epitope as Anti-HER3 Antibodies and Antigen-Binding Fragments Thereof

In another aspect, provided herein are HER3-binding molecules that bind to the same epitope as do the various anti-HER3 antibodies described herein. The term “epitope” as used herein refers to a protein determinant capable of binding to an antibody described herein. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. Such antibodies can be identified based on their ability to cross-compete (e.g., to competitively inhibit the binding of, in a statistically significant manner) with antibodies such as the CL16 antibody, the 2C2 antibody, or the 2C2-YTE mutant, in standard HER3 binding assays. Accordingly, in one aspect, provided herein are anti-HER3 antibodies and antigen-binding fragments thereof, e.g., human monoclonal antibodies, that compete for binding to HER3 with another anti-HER3 antibody or antigen-binding fragment thereof described herein, such as the CL16 antibody or the 2C2 antibody. The ability of a test antibody to inhibit the binding of, e.g., the CL16 antibody or the 2C2 antibody demonstrates that the test antibody can compete with that antibody for binding to HER3; such an antibody can, according to non-limiting theory, bind to the same or a related (e.g., a structurally similar or spatially proximal) epitope on HER3 as the anti-HER3 antibody or antigen-binding fragment thereof with which it competes. In one aspect, the anti-HER3 antibody or antigen-binding fragment thereof that binds to the same epitope on HER3 as, e.g., the CL16 antibody or the 2C2 antibody, is a human monoclonal antibody.

### IV. Mechanism of Action

In some aspects, a HER3-binding molecule, e.g., an anti-HER3 antibody or antigen-binding fragment thereof can suppress HER3 phosphorylation. In other aspects, a HER3-binding molecule, e.g., an anti-HER3 antibody or antigen-binding fragment thereof can suppress AKT phosphorylation. In still other aspects, a HER3-binding molecule, e.g., an anti-HER3 antibody or antigen-binding fragment thereof can suppress HER2-HER3 dimer formation. In some aspects, a HER3-binding molecule, e.g., an anti-HER3 antibody or antigen-binding fragment thereof can suppress cell growth. In some aspects, a HER3-binding molecule, e.g., an anti-HER3 antibody or antigen-binding fragment thereof lacks ADCC effect. In specific aspects, a HER3-binding molecule, e.g., an anti-HER3 antibody or antigen-binding fragment thereof can suppress HER3 phosphorylation, AKT phosphorylation, and/or tumor colony formation via a ligand-independent mechanism of action.

In some aspects, a HER3-binding molecule, e.g., an anti-HER3 antibody or antigen-binding fragment thereof can suppress HER3 phosphorylation in HRG-driven breast cancer MCF-7 cells as measured by ELISA, with an IC₅₀ lower than about 30 ng/mL, lower than about 25 ng/mL, lower than about 20 ng/mL, lower than about 15 ng/mL, or lower than about 10 ng/mL. In a specific aspect, a HER3-binding molecule, e.g., an anti-HER3 antibody or antigen-binding fragment thereof can suppress HER3 phosphorylation in HRG-driven breast cancer MCF-7 cells as measured by ELISA, with an IC₅₀ lower than about 20 ng/mL. In a specific aspect, a HER3-binding molecule, e.g., an anti-HER3 antibody or antigen-binding fragment thereof can suppress HER3 phosphorylation in HRG-driven breast cancer MCF-7 cells as measured by ELISA, with an IC₅₀ lower than about 15 ng/mL. In another specific aspect, a HER3-binding molecule, e.g., an anti-HER3 antibody or antigen-binding fragment thereof can suppress HER3 phosphorylation in HRG-driven breast cancer MCF-7 cells as measured by ELISA, with an IC₅₀ lower than about 10 ng/mL.

In some aspects, a HER3-binding molecule, e.g., an anti-HER3 antibody or antigen-binding fragment thereof can suppress cell growth in MDA-MB-175 breast cancer cells with an IC₅₀ lower than about 0.90 μg/mL, lower than about 0.80 μg/mL, lower than about 0.70 μg/mL, lower than about 0.60 μg/mL, lower than about 0.50 μg/mL, lower than about 0.40 μg/mL, lower than about 0.30 μg/mL, or lower than about 0.20 μg/mL. In a specific aspect, a HER3-binding molecule, e.g., an anti-HER3 antibody or antigen-binding fragment thereof can suppress cell growth in MDA-MB-175 breast cancer cells, with an IC₅₀ lower than about 0.50 μg/mL. In a specific aspect, a HER3-binding molecule, e.g., an anti-HER3 antibody or antigen-binding fragment thereof can suppress cell growth in MDA-MB-175 breast cancer cells, with an IC₅₀ lower than about 0.40 μg/mL. In another specific aspect, a HER3-binding molecule, e.g., an anti-HER3 antibody or antigen-binding fragment thereof can suppress cell growth in MDA-MB-175 breast cancer cells, with an IC₅₀ lower than about 0.30 μg/mL. In another specific aspect, a HER3-binding molecule, e.g., an anti-HER3 antibody or antigen-binding fragment thereof can suppress cell growth in MDA-MB-175 breast cancer cells, with an IC₅₀ lower than about 0.20 μg/mL.

In some aspects, a HER3-binding molecule, e.g., an anti-HER3 antibody or antigen-binding fragment thereof can suppress cell growth in HMCB melanoma cells with an IC₅₀ lower than about 0.20 μg/mL, lower than about 0.15 μg/mL, lower than about 0.10 μg/mL, lower than about 0.05 μg/mL, lower than about 0.04 μg/mL, or lower than about 0.03 μg/mL. In a specific aspect, a HER3-binding molecule, e.g., an anti-HER3 antibody or antigen-binding fragment thereof can suppress cell growth in HMCB melanoma cells, with an IC₅₀ lower than about 0.10 μg/mL. In a specific aspect, a HER3-binding molecule, e.g., an anti-HER3 antibody or antigen-binding fragment thereof can suppress cell growth in HMCB melanoma cells, with an IC₅₀ lower than about 0.05 μg/mL. In a specific aspect, a HER3-binding molecule, e.g., an anti-HER3 antibody or antigen-binding fragment thereof can suppress cell growth in HMCB melanoma cells, with an IC₅₀ lower than about 0.04 μg/mL. In a specific aspect, a HER3-binding molecule, e.g., an anti-HER3 antibody or antigen-binding fragment thereof can suppress cell growth in HMCB melanoma cells, with an IC₅₀ lower than about 0.03 μg/mL.

In some aspects, a HER3-binding molecule, e.g., an anti-HER3 antibody or antigen-binding fragment thereof can suppress HER3 phosphorylation in EGFR-driven HCC827 lung cancer cells with an IC₅₀ lower than about 20 ng/mL, lower than about 15 ng/mL, lower than about 10 ng/mL, lower than about 8 ng/mL, lower than about 6 ng/mL, lower than about 4 ng/mL, or lower than about 2 ng/mL. In a specific aspect, a HER3-binding molecule, e.g., an anti-HER3 antibody or antigen-binding fragment thereof can suppress HER3 phosphorylation in EGFR-driven HCC827 lung cancer cells, with an IC₅₀ lower than about 10 ng/mL. In a specific aspect, a HER3-binding molecule, e.g., an anti-HER3 antibody or antigen-binding fragment thereof can suppress HER3 phosphorylation in EGFR-driven HCC827 lung cancer cells, with an IC₅₀ lower than about 8 ng/mL. In a specific aspect, a HER3-binding molecule, e.g., an anti-HER3 antibody or antigen-binding fragment thereof can suppress HER3 phosphorylation in EGFR-driven HCC827 lung cancer cells, with an IC₅₀ lower than about 6 ng/mL. In a specific aspect, a HER3-binding molecule, e.g., an anti-HER3 antibody or antigen-binding fragment thereof can suppress HER3 phosphorylation in EGFR-driven HCC827 lung cancer cells, with an IC₅₀ lower than about 4 ng/mL. In a specific aspect, a HER3-binding molecule, e.g., an anti-HER3 antibody or antigen-binding fragment thereof can suppress HER3 phosphorylation in EGFR-driven HCC827 lung cancer cells, with an IC₅₀ lower than about 2 ng/mL.

In some aspects, a HER3-binding molecule, e.g., an anti-HER3 antibody or antigen-binding fragment thereof can suppress HER3 phosphorylation in EGFR-driven HCC827 lung cancer cells resistant to TKI with an IC₅₀ lower than about 30 ng/mL, lower than about 25 ng/mL, lower than about 20 ng/mL, lower than about 15 ng/mL, lower than about 10 ng/mL, or lower than about 5 ng/mL. In a specific aspect, a HER3-binding molecule, e.g., an anti-HER3 antibody or antigen-binding fragment thereof can suppress HER3 phosphorylation in EGFR-driven HCC827 lung cancer cells resistant to TKI, with an IC₅₀ lower than about 20 ng/mL. In a specific aspect, a HER3-binding molecule, e.g., an anti-HER3 antibody or antigen-binding fragment thereof can suppress HER3 phosphorylation in EGFR-driven HCC827 lung cancer cells resistant to TKI, with an IC₅₀ lower than about 15 ng/mL. In a specific aspect, a HER3-binding molecule, e.g., an anti-HER3 antibody or antigen-binding fragment thereof can suppress HER3 phosphorylation in EGFR-driven HCC827 lung cancer cells resistant to TKI, with an IC₅₀ lower than about 10 ng/mL. In a specific aspect, a HER3-binding molecule, e.g., an anti-HER3 antibody or antigen-binding fragment thereof can suppress HER3 phosphorylation in EGFR-driven HCC827 lung cancer cells resistant to TKI, with an IC₅₀ lower than about 5 ng/mL.

In some specific aspects, a HER3-binding molecule, e.g., an anti-HER3 antibody or antigen-binding fragment thereof can be used to treat TKI resistant cancers.

In some aspects, a HER3-binding molecule, e.g., an anti-HER3 antibody or antigen-binding fragment thereof can suppress HER3 phosphorylation in cMET-driven MKN45 human gastric adenocarcinoma cells with an IC₅₀ is lower than about 15 ng/mL, lower than about 10 ng/mL, lower than about 9 ng/mL, lower than about 8 ng/mL, lower than about 7 ng/mL, lower than about 6 ng/mL, lower than about 5 ng/mL, or lower than about 4 ng/mL. In a specific aspect, a HER3-binding molecule, e.g., an anti-HER3 antibody or antigen-binding fragment thereof can suppress HER3 phosphorylation in cMET-driven MKN45 human gastric adenocarcinoma cells with an IC₅₀ lower than about 10 ng/mL. In a specific aspect, a HER3-binding molecule, e.g., an anti-HER3 antibody or antigen-binding fragment thereof can suppress HER3 phosphorylation in cMET-driven MKN45 human gastric adenocarcinoma cells with an IC₅₀ lower than about 8 ng/mL. In a specific aspect, a HER3-binding molecule, e.g., an anti-HER3 antibody or antigen-binding fragment thereof can suppress HER3 phosphorylation in cMET-driven MKN45 human gastric adenocarcinoma cells with an IC₅₀ lower than about 6 ng/mL. In a specific aspect, a HER3-binding molecule, e.g., an anti-HER3 antibody or antigen-binding fragment thereof can suppress HER3 phosphorylation in cMET-driven MKN45 human gastric adenocarcinoma cells with an IC₅₀ lower than about 4 ng/mL.

In some aspects, a HER3-binding molecule, e.g., an anti-HER3 antibody or antigen-binding fragment thereof described herein can suppress pAKT in cMET-driven MKN45 cells with an IC₅₀ lower than about 15 ng/mL, lower than about 10 ng/mL, lower than about 9 ng/mL, lower than about 8 ng/mL, lower than about 7 ng/mL, lower than about 6 ng/mL, lower than about 5 ng/mL, lower than about 4 ng/mL, or lower than about 3 ng/mL. In a specific aspect, a HER3-binding molecule, e.g., an anti-HER3 antibody or antigen-binding fragment thereof can suppress pAKT in cMET-driven MKN45 cells with an IC₅₀ lower than about 8 ng/mL. In a specific aspect, a HER3-binding molecule, e.g., an anti-HER3 antibody or antigen-binding fragment thereof can suppress pAKT in cMET-driven MKN45 cells with an IC₅₀ lower than about 6 ng/mL. In a specific aspect, a HER3-binding molecule, e.g., an anti-HER3 antibody or antigen-binding fragment thereof can suppress pAKT in cMET-driven MKN45 cells with an IC₅₀ lower than about 4 ng/mL. In a specific aspect, a HER3-binding molecule, e.g., an anti-HER3 antibody or antigen-binding fragment thereof can suppress pAKT in cMET-driven MKN45 cells with an IC₅₀ lower than about 3 ng/mL.

In some aspects, a HER3-binding molecule, e.g., an anti-HER3 antibody or antigen-binding fragment thereof described herein can suppress pHER in FGFR2-driven Kato III human gastric signet ring carcinoma cells with an IC₅₀ lower than about 9 ng/mL, lower than about 8 ng/mL, lower than about 7 ng/mL, lower than about 6 ng/mL, lower than about 5 ng/mL, lower than about 4 ng/mL, lower than about 3 ng/mL, lower than about 2 ng/mL, or lower than about 1 ng/mL. In a specific aspect, a HER3-binding molecule, e.g., an anti-HER3 antibody or antigen-binding fragment thereof can suppress pHER in FGFR2-driven Kato III human gastric signet ring carcinoma cells with an IC₅₀ lower than about 5 ng/mL. In a specific aspect, a HER3-binding molecule, e.g., an anti-HER3 antibody or antigen-binding fragment thereof can suppress pHER in FGFR2-driven Kato III human gastric signet ring carcinoma cells with an IC₅₀ lower than about 4 ng/mL. In a specific aspect, a HER3-binding molecule, e.g., an anti-HER3 antibody or antigen-binding fragment thereof can suppress pHER in FGFR2-driven Kato III human gastric signet ring carcinoma cells with an IC₅₀ lower than about 3 ng/mL. In a specific aspect, a HER3-binding molecule, e.g., an anti-HER3 antibody or antigen-binding fragment thereof can suppress pHER in FGFR2-driven Kato III human gastric signet ring carcinoma cells with an IC₅₀ lower than about 2 ng/mL. In a specific aspect, a HER3-binding molecule, e.g., an anti-HER3 antibody or antigen-binding fragment thereof can suppress pHER in FGFR2-driven Kato III human gastric signet ring carcinoma cells with an IC₅₀ lower than about 1 ng/mL.

In some aspects, a HER3-binding molecule, e.g., an anti-HER3 antibody or antigen-binding fragment thereof can suppress pAKT in FGFR-2 driven Kato III cells with an IC₅₀ lower than about 6 ng/mL, lower than about 5 ng/mL, lower than about 4 ng/mL, lower than about 3 ng/mL, lower than about 2 ng/mL, or lower than about 1 ng/mL. In a specific aspect, a HER3-binding molecule, e.g., an anti-HER3 antibody or antigen-binding fragment thereof can suppress pAKT in FGFR-2 driven Kato III cells with an IC₅₀ lower than about 4 ng/mL. In a specific aspect, a HER3-binding molecule, e.g., an anti-HER3 antibody or antigen-binding fragment thereof can suppress pAKT in FGFR-2 driven Kato III cells with an IC₅₀ lower than about 3 ng/mL. In a specific aspect, a HER3-binding molecule, e.g., an anti-HER3 antibody or antigen-binding fragment thereof can suppress pAKT in FGFR-2 driven Kato III cells with an IC₅₀ lower than about 2 ng/mL. In a specific aspect, a HER3-binding molecule, e.g., an anti-HER3 antibody or antigen-binding fragment thereof can suppress pAKT in FGFR-2 driven Kato III cells with an IC₅₀ lower than about 1 ng/mL.

In some aspects, a HER3-binding molecule, e.g., an anti-HER3 antibody or antigen-binding fragment thereof described herein can suppress pHER in ligand independent BT-474 breast cancer cells with an IC₅₀ lower than about 10 ng/mL, lower than about 9 ng/mL, lower than about 8 ng/mL, lower than about 7 ng/mL, lower than about 6 ng/mL, lower than about 5 ng/mL, lower than about 4 ng/mL. In a specific aspect, a HER3-binding molecule, e.g., an anti-HER3 antibody or antigen-binding fragment thereof can suppress pHER in ligand independent BT-474 breast cancer cells with an IC₅₀ lower than about 8 ng/mL. In a specific aspect, a HERS-binding molecule, e.g., an anti-HER3 antibody or antigen-binding fragment thereof can suppress pHER in ligand independent BT-474 breast cancer cells with an IC₅₀ lower than about 6 ng/mL. In a specific aspect, a HER3-binding molecule, e.g., an anti-HER3 antibody or antigen-binding fragment thereof can suppress pHER in ligand independent BT-474 breast cancer cells with an IC₅₀ lower than about 4 ng/mL.

In some aspects, a HER3-binding molecule, e.g., an anti-HER3 antibody or antigen-binding fragment thereof described herein can suppress pAKT in ligand independent BT-474 breast cancer cells with an IC₅₀ lower than about 10 ng/mL, lower than about 9 ng/mL, lower than about 8 ng/mL, lower than about 7 ng/mL, lower than about 6 ng/mL, lower than about 5 ng/mL, lower than about 4 ng/mL. In a specific aspect, a HER3-binding molecule, e.g., an anti-HER3 antibody or antigen-binding fragment thereof can suppress pAKT in ligand independent BT-474 breast cancer cells with an IC₅₀ lower than about 8 ng/mL. In a specific aspect, a HER3-binding molecule, e.g., an anti-HER3 antibody or antigen-binding fragment thereof can suppress pAKT in ligand independent BT-474 breast cancer cells with an IC₅₀ lower than about 6 ng/mL. In a specific aspect, a HER3-binding molecule, e.g., an anti-HER3 antibody or antigen-binding fragment thereof can suppress pAKT in ligand independent BT-474 breast cancer cells with an IC₅₀ lower than about 4 ng/mL. In some aspects, a HER3-binding molecule, e.g., an anti-HER3 antibody or antigen-binding fragment thereof can suppress pHER3, pAKT, and tumor colony formation in BT-474 cells, a ligand independent breast cancer model.

In some aspects, a HER3-binding molecule, e.g., an anti-HER3 antibody or antigen-binding fragment thereof described herein can suppress HRG induced VEGF secretion. In a specific aspect, a HER3-binding molecule, e.g., an anti-HER3 antibody or antigen-binding fragment thereof described herein can suppress HRG induced VEGF secretion in ligand independent BT-474 breast cancer cells and/or HRG-driven breast cancer MCF-7 cells.

In some aspects, a HER3-binding molecule, e.g., an anti-HER3 antibody or antigen-binding fragment thereof described herein can cause cell cycle arrest. In a specific aspect, a HER3-binding molecule, e.g., an anti-HER3 antibody or antigen-binding fragment thereof described herein can cause cell cycle arrest in breast cancer cells, including but not limited to SKBR3 or BT474 cells.

### V. Preparation of Anti-HERS Antibodies and Antigen-Binding Fragments

Monoclonal anti-HER3 antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. radioimmunoassay (RIA); enzyme-linked immunosorbent assay (ELISA)) can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively anti-HER3 monoclonal antibodies can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant anti-HER3 monoclonal antibodies or antigen-binding fragments thereof of the desired species can be isolated from phage display libraries expressing CDRs of the desired species as described (McCafferty et al., 1990, Nature, 348:552-554; Clarkson et al., 1991, Nature, 352:624-628; and Marks et al., 1991, J. Mol. Biol., 222:581-597).

The polynucleotide(s) encoding an anti-HER3 antibody or antigen-binding fragments thereof can further be modified in a number of different manners using recombinant DNA technology to generate alternative antibodies. In some aspects, the constant domains of the light and heavy chains of, for example, a mouse monoclonal antibody can be substituted (1) for those regions of, for example, a human antibody to generate a chimeric antibody or (2) for a non-immunoglobulin polypeptide to generate a fusion antibody. In some aspects, the constant regions are truncated or removed to generate the desired antibody fragment of a monoclonal antibody. Site-directed or high-density mutagenesis of the variable region can be used to optimize specificity, affinity, etc. of a monoclonal antibody.

In certain aspects, the anti-HER3 antibody or antigen-binding fragment thereof is a human antibody or antigen-binding fragment thereof. Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., 1991, J. Immunol., 147 (1):86-95; and U.S. Pat. No. 5,750,373).

Also, the anti-HER3 human antibody or antigen-binding fragment thereof can be selected from a phage library, where that phage library expresses human antibodies, as described, for example, in Vaughan et al., 1996, Nat. Biotech., 14:309-314, Sheets et al., 1998, Proc. Nat'l. Acad. Sci., 95:6157-6162, Hoogenboom and Winter, 1991, J. Mol. Biol., 227:381, and Marks et al., 1991, J. Mol. Biol., 222:581). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., 2007, J. Mol. Bio., doi:10.1016/j.jmb.2007.12.018 (each of which is incorporated by reference in its entirety).

Affinity maturation strategies and chain shuffling strategies (Marks et al., 1992, Bio/Technology 10:779-783, incorporated by reference in its entirety) are known in the art and can be employed to generate high affinity human antibodies or antigen-binding fragments thereof.

In some aspects, an anti-HER3 monoclonal antibody can be a humanized antibody. Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence. Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing HER3 binding. Accordingly, part or all of the non-human or human CDR sequences are maintained while the non-human sequences of the variable and constant regions can be replaced with human or other amino acids.

Antibodies can also optionally be humanized, resurfaced, engineered or human antibodies engineered with retention of high affinity for the antigen HER3 and other favorable biological properties. To achieve this goal, humanized (or human) or engineered anti-HER3 antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as HER3. In this way, framework (FW) residues can be selected and combined from the consensus and import sequences so that the desired antibody characteristic, such as increased affinity for the target antigen(s), is achieved.

Humanization, resurfacing or engineering of anti-HER3 antibodies or antigen-binding fragments thereof described herein can be performed using any known method, such as but not limited to those described in, Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; International Application Nos. PCT/US98/16280; PCT/US96/18978; PCT/US91/09630; PCT/US91/05939; PCT/US94/01234; PCT/GB89/01334; PCT/GB91/01134; PCT/GB92/01755; International Patent Application Publication Nos. WO90/14443; WO90/14424; WO90/14430; and European Patent Publication No. EP 229246; each of which is entirely incorporated herein by reference, including the references cited therein.

Anti-HER3 humanized antibodies and antigen-binding fragments thereof can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain aspects an anti-HER3 antibody fragment is provided. Various techniques are known for the production of antibody fragments. Traditionally, these fragments are derived via proteolytic digestion of intact antibodies (for example Morimoto et al., 1993, Journal of Biochemical and Biophysical Methods 24:107-117; Brennan et al., 1985, Science, 229:81). In certain aspects, anti-HER3 antibody fragments are produced recombinantly. Fab, Fv, and scFv antibody fragments can all be expressed in and secreted from E. coli or other host cells, thus allowing the production of large amounts of these fragments. Such anti-HER3 antibody fragments can also be isolated from the antibody phage libraries discussed above. The anti-HER3 antibody fragments can also be linear antibodies as described in U.S. Pat. No. 5,641,870. Other techniques for the production of antibody fragments will be apparent to the skilled practitioner.

Techniques can be adapted for the production of single-chain antibodies specific to HER3 (see, e.g., U.S. Pat. No. 4,946,778). In addition, methods can be adapted for the construction of Fab expression libraries (see, e.g., Huse et al., Science 246:1275-1281 (1989)) to allow rapid and effective identification of monoclonal Fab fragments with the desired specificity for HER3, or derivatives, fragments, analogs or homologs thereof. Antibody fragments can be produced by techniques in the art including, but not limited to: (a) a F(ab′)2 fragment produced by pepsin digestion of an antibody molecule; (b) a Fab fragment generated by reducing the disulfide bridges of an F(ab′)2 fragment, (c) a Fab fragment generated by the treatment of the antibody molecule with papain and a reducing agent, and (d) Fv fragments.

It can further be desirable, especially in the case of antibody fragments, to modify an anti-HER3 antibody or antigen-binding fragment thereof in order to increase its serum half-life. This can be achieved, for example, by incorporation of a salvage receptor binding epitope into the antibody or antibody fragment by mutation of the appropriate region in the antibody or antibody fragment or by incorporating the epitope into a peptide tag that is then fused to the antibody or antibody fragment at either end or in the middle (e.g., by DNA or peptide synthesis), or by YTE mutation. Other methods to increase the serum half-life of an antibody or antigen-binding fragment thereof, e.g., conjugation to a heterologous molecule such as PEG are known in the art.

Also provided herein are heteroconjugate anti-HER3 antibodies and antigen-binding fragments thereof. Heteroconjugate antibodies are composed of two covalently joined antibodies. Such antibodies have, for example, been proposed to target immune cells to unwanted cells (see, e.g., U.S. Pat. No. 4,676,980). It is contemplated that the heteroconjugate anti-HER3 antibodies and antigen-binding fragments thereof can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

In certain aspects, the HER3-binding molecules described herein, e.g., antibodies or antigen-binding fragments thereof can be combined with other therapeutic agents or conjugated to other therapeutic agents or toxins to form immunoconjugates and/or fusion proteins. Examples of such therapeutic agents and toxins include, but are not limited to cetuximab (Erbitux®), panitumumab (Vectibix®), lapatinib (Tykerb®/Tyverb®), and paclitaxel (Taxol®, Abraxane®) and derivatives (e.g., docetaxel).

In some aspects the HER3-binding molecules described herein, e.g., antibodies or antigen-binding fragments thereof can be conjugated to antibodies or antibody fragments targeting epidermal growth factor receptor (EGFR). In other aspects, the HER3-binding molecules described herein can be conjugated to tyrosine kinase inhibitors. In some specific aspects, the HER3-binding molecules described herein can be conjugated to inhibitors of the tyrosine kinase activity associated with EGFR and/or HER2/neu. In some aspects, the HER3-binding molecules described herein can be conjugated to antimitotic agents. In some specific aspects, the HER3-binding molecules described herein can be conjugated to agents that stabilize the mitotic spindle microtubule assembly.

For the purposes provided herein, it should be appreciated that modified anti-HER3 antibodies or antigen-binding fragments thereof can comprise any type of variable region that provides for the association of the antibody or polypeptide with HER3. In this regard, the variable region can comprise or be derived from any type of mammal that can be induced to mount a humoral response and generate immunoglobulins against the desired tumor associated antigen. As such, the variable region of the modified anti-HER3 antibodies or antigen-binding fragments thereof can be, for example, of human, murine, non-human primate (e.g., cynomolgus monkeys, macaques, etc.) or lupine origin. In some aspects both the variable and constant regions of the modified anti-HER3 antibodies or antigen-binding fragments thereof are human. In other aspects the variable regions of compatible antibodies (usually derived from a non-human source) can be engineered or specifically tailored to improve the binding properties or reduce the immunogenicity of the molecule. In this respect, variable regions can be humanized or otherwise altered through the inclusion of imported amino acid sequences.

In certain aspects, the variable domains in both the heavy and light chains of an anti-HER3 antibody or antigen-binding fragment thereof are altered by at least partial replacement of one or more CDRs and, if necessary, by partial framework region replacement and sequence changing. Although the CDRs can be derived from an antibody of the same class or even subclass as the antibody from which the framework regions are derived, it is envisaged that the CDRs will be derived from an antibody of different class and in certain aspects from an antibody from a different species. It is not necessary to replace all of the CDRs with the complete CDRs from the donor variable region to transfer the antigen binding capacity of one variable domain to another. Rather, it is only necessary to transfer those residues that are necessary to maintain the activity of the antigen binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that the modified anti-HER3 antibodies or antigen-binding fragments thereof described herein will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In some aspects, the constant region of the modified antibodies will comprise a human constant region. Modifications to the constant region compatible with the antibodies described herein comprise additions, deletions or substitutions of one or more amino acids in one or more domains. That is, the modified antibodies disclosed herein can comprise alterations or modifications to one or more of the three heavy chain constant domains (CH1, CH2 or CH3) and/or to the light chain constant domain (CL). In some aspects, modified constant regions wherein one or more domains are partially or entirely deleted are contemplated. In some aspects, the modified antibodies will comprise domain deleted constructs or variants wherein the entire CH2 domain has been removed (ΔCH2 constructs). In some aspects, the omitted constant region domain will be replaced by a short amino acid spacer (e.g., 10 residues) that provides some of the molecular flexibility typically imparted by the absent constant region.

Besides their configuration, it is known in the art that the constant region mediates several effector functions. For example, binding of the C1 component of complement to antibodies activates the complement system. Activation of complement is important in the opsonisation and lysis of cell pathogens. The activation of complement also stimulates the inflammatory response and can also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In certain aspects, an anti-HER3 antibody or an antigen-binding fragment thereof provides for altered effector functions that, in turn, affect the biological profile of the administered antibody or antigen-binding fragment thereof. For example, the deletion or inactivation (through point mutations or other means) of a constant region domain can reduce Fc receptor binding of the circulating modified antibody thereby increasing tumor localization. In other cases it can be that constant region modifications moderate complement binding and thus reduce the serum half-life and nonspecific association of a conjugated cytotoxin. Yet other modifications of the constant region can be used to eliminate disulfide linkages or oligosaccharide moieties that allow for enhanced localization due to increased antigen specificity or antibody flexibility. Similarly, modifications to the constant region can be made using well known biochemical or molecular engineering techniques well within the purview of the skilled artisan.

In certain aspects, a HER3-binding molecule that is an antibody or antigen-binding fragment thereof does not have one or more effector functions. For instance, in some aspects, the antibody or antigen-binding fragment thereof has no antibody-dependent cellular cytotoxicity (ADCC) activity and/or no complement-dependent cytotoxicity (CDC) activity. In certain aspects, the anti-HER3 antibody or antigen binding fragment thereof does not bind to an Fc receptor and/or complement factors. In certain aspects, the antibody or antigen-binding fragment thereof has no effector function.

It will be noted that in certain aspects, the anti-HER3 modified antibodies or antigen-binding fragments thereof can be engineered to fuse the CH3 domain directly to the hinge region of the respective modified antibodies or fragments thereof. In other constructs it can be desirable to provide a peptide spacer between the hinge region and the modified CH2 and/or CH3 domains. For example, compatible constructs could be expressed wherein the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain aspects, any spacer added to the construct will be relatively non-immunogenic, or even omitted altogether, so as to maintain the desired biochemical qualities of the modified antibodies.

Besides the deletion of whole constant region domains, it will be appreciated that the anti-HER3 antibodies and antigen-binding fragments thereof described herein can be provided by the partial deletion or substitution of a few or even a single amino acid. For example, the mutation of a single amino acid in selected areas of the CH2 domain can be enough to substantially reduce Fc binding and thereby increase tumor localization. Similarly, it can be desirable to simply delete that part of one or more constant region domains that control the effector function (e.g., complement C1Q binding) to be modulated. Such partial deletions of the constant regions can improve selected characteristics of the antibody or antigen-binding fragment thereof (e.g., serum half-life) while leaving other desirable functions associated with the subject constant region domain intact. Moreover, as alluded to above, the constant regions of the disclosed anti-HER3 antibodies and antigen-binding fragments thereof can be modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified antibody or antigen-binding fragment thereof. Certain aspects can comprise the addition of one or more amino acids to the constant region to enhance desirable characteristics such as decreasing or increasing effector function or provide for more cytotoxin or carbohydrate attachment. In such aspects it can be desirable to insert or replicate specific sequences derived from selected constant region domains.

Also provided herein are variants and equivalents which are substantially homologous to the chimeric, humanized and human anti-HER3 antibodies, or antigen-binding fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

An anti-HER3 antibody or antigen-binding fragment thereof can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in Remington's Pharmaceutical Sciences, 20th ed., Mack Publishing Co., Easton, Pa. (2000).

### VI. Polynucleotides Encoding HER3-Binding Molecules

In certain aspects, provided herein are polynucleotides comprising nucleic acid sequences that encode a polypeptide that specifically binds HER3 or an antigen-binding fragment thereof. For example, provided herein is a polynucleotide comprising a nucleic acid sequence that encodes an anti-HER3 antibody or encodes an antigen-binding fragment of such an antibody. The polynucleotides described herein can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain aspects, the polynucleotides are isolated. In certain aspects, the polynucleotides are substantially pure. In certain aspects the polynucleotides comprise the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for an HER3-binding proprotein which is the mature protein plus additional 5′ amino acid residues.

In certain aspects the polynucleotides comprise the coding sequence for the mature HER3-binding polypeptide, e.g., an anti-HER3 antibody or an antigen-binding fragment thereof fused in the same reading frame to a marker sequence that allows, for example, for purification of the encoded polypeptide. For example, the marker sequence can be a hexahistidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used.

Further provided herein are variants of the described polynucleotides encoding, for example, HER3-binding fragments, analogs, and derivatives of the HER3-binding molecules described herein.

The polynucleotide variants can contain alterations in the coding regions, non-coding regions, or both. In some aspects the polynucleotide variants contain alterations which produce silent substitutions, additions, or deletions, but do not alter the properties or activities of the encoded polypeptide. In some aspects, nucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli). Vectors and cells comprising the polynucleotides described herein are also provided.

In some aspects a DNA sequence encoding a HER3-binding molecule, e.g., an anti-HER3 antibody or an antigen-binding fragment thereof can be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest will be inserted into an expression vector and operatively linked to an expression control sequence appropriate for expression of the protein in a desired host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

In certain aspects, recombinant expression vectors are used to amplify and express DNA encoding anti-HER3 antibodies or antigen-binding fragments thereof. Recombinant expression vectors are replicable DNA constructs which have synthetic or cDNA-derived DNA fragments encoding a polypeptide chain of an anti-HER3 antibody or an antigen-binding fragment thereof, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papillomavirus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

Suitable host cells for expression of an HER3-binding molecule, e.g., an anti-HER3 antibody or antigen-binding fragment thereof include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin as described below. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems can also be advantageously employed to express recombinant HER3-binding molecules, e.g., anti-HER3 antibodies or antigen-binding fragments thereof. Expression of recombinant proteins in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175, 1981), and other cell lines including, for example, L cells, C127, 3T3, Chinese hamster ovary (CHO), NSO, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, BioTechnology 6:47 (1988).

HER3-binding molecules, e.g., anti-HER3 antibodies or antigen-binding fragments thereof produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify an HER3-binding molecule. Some or all of the foregoing purification steps, in various combinations, can also be employed to provide a homogeneous recombinant protein.

A recombinant HER3-binding protein, e.g., an anti-HER3 antibody or antigen-binding fragment thereof produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

In certain aspects, the HER3-binding molecule is a polypeptide that is not an antibody. A variety of methods for identifying and producing non-antibody polypeptides that bind with high affinity to a protein target are known in the art. See, e.g., Skerra, Curr. Opin. Biotechnol., 18:295-304 (2007), Hosse et al., Protein Science, 15:14-27 (2006), Gill et al., Curr. Opin. Biotechnol., 17:653-658 (2006), Nygren, FEBS J., 275:2668-76 (2008), and Skerra, FEBS J., 275:2677-83 (2008), each of which is incorporated by reference herein in its entirety. In certain aspects, phage display technology can been used to identify/produce an HER3-binding polypeptide. In certain aspects, the polypeptide comprises a protein scaffold of a type selected from the group consisting of protein A, a lipocalin, a fibronectin domain, an ankyrin consensus repeat domain, and thioredoxin.

### VI. Treatment Methods Using Therapeutic Anti-HER3 Antibodies

Methods provided herein are directed to the use of anti-HER3 binding molecules, e.g., antibodies, including antigen-binding fragments, variants, and derivatives thereof, to treat patients having a disease associated with HER3 expression or HER3-expressing cells, such as cancer (e.g., melanoma, head and neck cancer, lung cancer such as non-small cell lung cancer, colon cancer, breast cancer, pancreatic cancer, thyroid cancer, or gastric cancer). By “HER3-expressing cell” is meant a cell expressing HER3. Methods for detecting HER3 expression in cells are well known in the art and include, but are not limited to, PCR techniques, immunohistochemistry, flow cytometry, Western blot, ELISA, and the like.

Though the following discussion refers to diagnostic methods and treatment of various diseases and disorders with an HER3-binding molecule described herein, the methods described herein are also applicable to anti-HER3 antibodies (e.g., 2C2-YTE), and the antigen-binding fragments, variants, and derivatives of these anti-HER3 antibodies that retain the desired properties of the anti-HER3 antibodies described herein, e.g., capable of specifically binding HER3 and neutralizing HER3 activity. In some aspects, HER3-binding molecules are human or humanized antibodies that do not mediate human ADCC, or are selected from known anti-HER3 antibodies that do not mediate ADCC, or are anti-HER3 antibodies that are engineered such that they do not mediate ADCC. In some aspects, the HER3-binding molecule is a clone 16 monoclonal antibody. In other aspects, the HER3-binding molecule is a clone 16 YTE mutant antibody. In some aspects the HER3-binding molecule is a P2B11 monoclonal antibody. In some aspects the HER3-binding molecule is a 1A4 monoclonal antibody. In some aspects the HER3-binding molecule is a 2C2 monoclonal antibody. In some aspects the HER3-binding molecule is a 2F10 monoclonal antibody. In some aspects the HER3-binding molecule is a 3E1 monoclonal antibody. In some aspects the HER3-binding molecule is a P2B11 monoclonal antibody engineered to extend serum half-life. In some aspects the HER3-binding molecule is a 1A4 monoclonal antibody engineered to extend serum half-life. In some aspects the HER3-binding molecule is a 2C2 monoclonal antibody engineered to extend serum half-life. In some aspects the HER3-binding molecule is a 2F10 monoclonal antibody engineered to extend serum half-life. In some aspects the HER3-binding molecule is a 3E1 monoclonal antibody engineered to extend serum half-life. In other aspects the HER3-binding molecule is a P2B11 YTE mutant antibody. In other aspects the HER3-binding molecule is a 1A4 YTE mutant antibody. In other aspects the HER3-binding molecule is a 2C2-YTE mutant antibody. In other aspects the HER3-binding molecule is a 2F10 YTE mutant antibody. In other aspects the HER3-binding molecule is a 3E1 YTE mutant antibody.

In one aspect, treatment includes the application or administration of an anti-HER3 binding molecule, e.g., an antibody (e.g., 2C2-YTE) or antigen binding fragment, variant, or derivative thereof described herein to a subject or patient, or application or administration of the anti-HER3 binding molecule to an isolated tissue or cell line from a subject or patient, where the subject or patient has a disease, a symptom of a disease, or a predisposition toward a disease. In another aspect, treatment is also intended to include the application or administration of a pharmaceutical composition comprising the anti-HER3 binding molecule, e.g., an antibody (e.g., 2C2-YTE) or antigen binding fragment, variant, or derivative thereof of described herein to a subject or patient, or application or administration of a pharmaceutical composition comprising the anti-HER3 binding molecule to an isolated tissue or cell line from a subject or patient, who has a disease, a symptom of a disease (e.g., cancer), or a predisposition toward a disease (e.g., cancer).

The anti-HER3 binding molecules, e.g., antibodies (e.g., 2C2-YTE) or antigen-binding fragments, variants, or derivatives thereof described herein are useful for the treatment of various cancers. In one aspect, provided herein are anti-HER binding molecules, e.g., antibodies (e.g., 2C2-YTE) or antigen-binding fragments, variants, or derivatives thereof for use as a medicament, in particular for use in the treatment or prophylaxis of cancer. Examples of cancer include, but are not limited to colon or colorectal cancer, lung cancer (e.g., non-small cell lung cancer), gastric cancer, head and neck squamous cells cancer, melanoma, pancreatic cancer, prostate cancer, thyroid cancer, ovarian cancer, and breast cancer.

In accordance with the methods provided herein, at least one anti-HER3 binding molecule, e.g., an antibody (e.g., 2C2-YTE) or antigen binding fragment, variant, or derivative thereof as defined elsewhere herein is used to promote a positive therapeutic response with respect to cancer. The term “positive therapeutic response” with respect to cancer treatment refers to an improvement in the disease in association with the activity of these anti-HER3 binding molecules, e.g., antibodies or antigen-binding fragments, variants, or derivatives thereof, and/or an improvement in the symptoms associated with the disease. Thus, for example, an improvement in the disease can be characterized as a complete response. By “complete response” is intended an absence of clinically detectable disease with normalization of any previously test results. Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of an anti-HER3 binding molecule described herein. In specific aspects, such terms refer to one, two or three or more results following the administration of anti-HER3 binding molecules described herein: (1) a stabilization, reduction or elimination of the cancer cell population; (2) a stabilization or reduction in cancer growth; (3) an impairment in the formation of cancer; (4) eradication, removal, or control of primary, regional and/or metastatic cancer; (5) a reduction in mortality; (6) an increase in disease-free, relapse-free, progression-free, and/or overall survival, duration, or rate; (7) an increase in the response rate, the durability of response, or number of patients who respond or are in remission; (8) a decrease in hospitalization rate, (9) a decrease in hospitalization lengths, (10) the size of the cancer is maintained and does not increase or increases by less than 10%, preferably less than 5%, preferably less than 4%, preferably less than 2%, and (12) an increase in the number of patients in remission.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy with the anti-HER3 binding molecule, e.g., an antibody or antigen-binding fragment, variant, or derivative thereof, can experience the beneficial effect of an improvement in the symptoms associated with the disease.

The anti-HER3 binding molecules, e.g., antibodies (e.g., 2C2-YTE) or antigen-binding fragments, variants, or derivatives thereof described herein can be used in combination with another therapy, including any known therapies for cancer, such as any agent or combination of agents that are known to be useful, or which have been used or are currently in use, for treatment of cancer, e.g., colon or colorectal cancer, lung cancer, gastric cancer, head and neck squamous cells cancer, thyroid cancer, pancreatic cancer, ovarian cancer, and breast cancer. The second agent or combination of agents of the pharmaceutical combination formulation or dosing regimen preferably has complementary activities to the antibody or polypeptide described herein such that they do not adversely affect each other. In specific aspects, the agent for use in combination with anti-HER3 binding molecules, e.g., antibodies (e.g., 2C2-YTE) or antigen-binding fragments, variants, or derivatives thereof described herein, is an EGFR inhibitor (e.g., anti-EGFR antibody such as cetuximab or a reversible or irreversible small molecule inhibitor of EGFR such as erlotinib or AZD9291) or a HER2 inhibitor (e.g., anti-HER2 antibody such as trastuzumab). In specific aspects, the agent for use in combination with anti-HER3 binding molecules e.g., antibodies (e.g., 2C2-YTE) or antigen-binding fragments, variants, or derivatives thereof described herein, is an ErbB (e.g., EGFR, HER2, or HER3) inhibitor, such as necitumumab (IMC-11F8, Lilly), zalutumumab (Genmab), SYM-004 (Symphogen), KADCYLA® (ado-trastuzumab emtansine, Roche), IMG289, RO5083945 (GA201, Roche), AMG595 (Amgen), gefitinib (IRESSA®, AstraZeneca/Teva), afatinib (GILOTRIF®, Boehringer Ingelheim), rociletinib (CO-1686, Clovis), EGF816 (Novartis), neratinib (Puma Biotechnology), varlitinib (ARRY-334543, ASLAN001, Array BioPharma), ASP8273 (Astellas Pharma), PF-00299804 (Pfizer), icotinib (Betta Pharmaceuticals), poziotinib (NOV120101, Hanmi Pharmaceuticals), or vandetanib (AstraZeneca). In specific aspects, the agent for use in combination with anti-HER3 binding molecules, e.g., antibodies (e.g., 2C2-YTE) or antigen-binding fragments, variants, or derivatives thereof described herein, is a BRAF inhibitor such as vemurafenib (ZELBORAF®), dabrafenib (TAFINLAR®), encorafenib (LGX818, Novartis), PLX-4720, PLX-3603 (RO5212054, Roche/Genentech), PLX-8394 (Daiichi Sankyo), CEP-32496 (Ambit Biosciences), XL281 (BMS-908662, Exelixis), or RAF265 (CHIR-265, Novartis). In specific aspects, the agent for use in combination with anti-HER3 binding molecules, e.g., antibodies (e.g., 2C2-YTE) or antigen-binding fragments, variants, or derivatives thereof described herein, is a MEK (mitogen-activated protein kinase (MAPK) kinase, also known as MAPKK) inhibitor such as selumetinib (AZD6244, ARRY-142866, AstraZeneca), WX-554 (Wilex), trametinib (MEKINIST®; GlaxoSmithKline), refametinib (Ardea Biosciences), E-6201 (Eisai), MEK-162 (Novartis), cobimetinib (GDC-0973; XL-518; Exelixis, Roche), TAK-733 (Takeda Phamaceuticals), binimetinib (Array BioPharma), PD-0325901 (Pfizer), pimasertib (MSC1936369; EMD Serono), MSC2015103 (EMD Serono), WX-554 (WILEX), MEK162 (ARRY-162, Novartis), or RO48987655 (CH4987655; CIF/RG7167; Chugai Pharmaceuticals).). In specific aspects, the agent for use in combination with anti-HER3 binding molecules e.g., antibodies (e.g., 2C2-YTE) or antigen-binding fragments, variants, or derivatives thereof described herein, is a PI3K/AKT/mTOR pathway inhibitor, such as everolimus (Novartis), temsirolimus (Wyeth/Pfizer), PF-05212384 (Pfizer), PF-04691502 (Pfizer), BYL-719 (Novartis), BEZ235 (Novartis), SF1126 (SignalRx Pharmaceuticals), BKM120 (Novartis), PX-866 (Oncothyreon), GDC-0941 (Genentech), VS-5584 (Verastem), CUDC-907 (Curis), XL-147 (Sanofi), XL-765 (Sanofi), IPI-145 (Infinity Pharmaceuticals), copanlisib (BAY80-6946, Bayer), palomid 529 (Paloma Pharmaceuticals), LY2780301 (Eli Lilly), metformin, ZSTK474 (Zenyaku Kogyo), GSK2636771 (GlaxoSmithKline), or BGT226 (Novartis). In specific aspects, agents for use in combination with anti-HER3 binding molecules, e.g., antibodies (e.g., 2C2-YTE) or antigen-binding fragments, variants, or derivatives thereof described herein, are chemotherapeutic agents (e.g., platinums (such as cisplatin or oxaliplatin), antimetabolites, alkylating agents, topoisomerase inhibitors, or microtubule targeting agents).

In specific aspects, provided herein are methods for treating cancer (e.g., head and neck cancer (e.g., squamous cell carcinoma of the head and neck), breast cancer, colon or colorectal cancer, thyroid cancer, pancreatic cancer, lung cancer (e.g., non-small cell lung cancer), gastric cancer, ovarian cancer, or melanoma) comprising administering to a human subject in need thereof an amount of an anti-HER3 antibody or antigen binding fragment thereof described herein (e.g., antibody 2C2 or 2C2-YTE) sufficient to achieve a serum concentration (or serum PK level) equal to or greater than a target serum concentration (e.g., Cₘᵢₙ or trough concentration) in the subject throughout the dosing interval, for monotherapy or combination therapy, for example, in combination with a chemotherapeutic agent (e.g., platinum), a BRAF inhibitor (e.g., vemurafenib or dabrafenib), an EGFR inhibitor (e.g., an anti-EGFR antibody such as cetuximab or a reversible or irreversible inhibitor such as erlotinib or AZD9291) or a HER2 inhibitor (e.g., anti-HER2 antibody such as trastuzumab).

In particular aspects, the amount of an anti-HER3 antibody (e.g., 2C2-YTE) or an antigen binding fragment thereof administered to a human subject for treating cancer in accordance with the methods described herein, is an amount sufficient to achieve an antibody serum concentration equal to or higher than a target serum concentration (e.g., 50 μg/mL over a period of approximately 2 weeks, 3 weeks, or 4 weeks), for example throughout the dosing interval, in approximately 90% of subjects in a group of subjects being administered the anti-HER3 antibody (e.g., 2C2-YTE) or an antigen binding fragment thereof. In particular aspects, the amount of an anti-HER3 antibody (e.g., 2C2-YTE) or an antigen binding fragment thereof administered to a human subject for treating cancer in accordance with the methods described herein, is an amount sufficient to achieve an antibody serum concentration equal to or higher than a target serum concentration (e.g., 50 μg/mL over a period of approximately 2 weeks, 3 weeks, or 4 weeks), for example throughout the dosing interval, in a majority (e.g., approximately greater than 50%) of subjects in a group of subjects being administered the anti-HER3 antibody (e.g., 2C2-YTE) or an antigen binding fragment thereof. In specific aspects, the dosing interval is approximately 6 days, 7 days, 8 days, 13 days, 14 days, 15 days, 19 days, 20 days, 21 days, 22 days, 27 days, 28 days, or 29 days. In a certain aspect, the dosing interval is approximately 7 days (1 week), 14 days (2 weeks), or 21 days (3 weeks). In specific aspects, the dosing interval is approximately 21 days.

In specific aspects, disclosed herein is a method of treating cancer in a human subject in need thereof, comprising administering to the human subject an amount of an anti-HER3 antibody (e.g., 2C2-YTE) or antigen-binding fragment thereof which specifically binds to an epitope within the extracellular domain of HER3, wherein the amount of the anti-HER3 antibody or antigen-binding fragment thereof is sufficient to achieve:
(i) a Cₘᵢₙ of 50 μg/mL or greater antibody serum concentration; or
(ii) antibody serum concentration of 50 μg/mL or greater.

In particular embodiments, the amount of an anti-HER3 antibody (e.g., 2C2-YTE) or antigen-binding fragment thereof is sufficient to achieve:
(i) a Cₘᵢₙ of 50 μg/mL or greater antibody serum concentration; or
(ii) antibody serum concentration of 50 μg/mL or greater throughout the dosing interval.
   In specific aspects, the dosing interval is approximately 6 days, 7 days, 8 days, 13 days, 14 days, 15 days, 19 days, 20 days, 21 days, 22 days, 27 days, 28 days, or 29 days. In a certain aspect, the dosing interval is approximately 7 days (1 week), 14 days (2 weeks), or 21 days (3 weeks). In specific aspects, the dosing interval is approximately 21 days.

In a particular aspect, an amount of the anti-HER3 antibody or antigen-binding fragment thereof administered to an human subject is sufficient to achieve at least one of the following over a period of 1 week, 2 weeks, 3 weeks or 4 weeks: (i) a Cₘᵢₙ of 50 μg/mL or greater antibody serum concentration; and/or (ii) an antibody serum concentration of 50 μg/mL or greater. In a specific aspect, an amount of an anti-HER3 antibody (e.g., 2C2-YTE) or antigen-binding fragment thereof administered is sufficient to achieve (i) a Cₘᵢₙ of 50 μg/mL or greater antibody serum concentration, or (ii) an antibody serum concentration of 50 μg/mL or greater, over a period of at least 6 days, at least 7 days, at least 8 days, at least 12 days, at least 13 days, at least 14 days, at least 15, days, at least 19 days, or at least 20 days, following antibody administration. In a specific aspect, an amount of an anti-HER3 antibody (e.g., 2C2-YTE) or antigen-binding fragment thereof administered is sufficient to achieve (i) a Cₘᵢₙ of 50 μg/mL or greater antibody serum concentration, or (ii) an antibody serum concentration of 50 μg/mL or greater, over a period of at least 12 days, at least 13 days, at least 14 days, or at least 15 days, following antibody administration. In a specific aspect, an amount of an anti-HER3 antibody (e.g., 2C2-YTE) or antigen-binding fragment thereof administered is sufficient to achieve (i) a Cₘᵢₙ of 50 μg/mL or greater antibody serum concentration, or (ii) an antibody serum concentration of 50 μg/mL or greater, over a period of at least 18 days, at least 19 days, or at least 20 days, following antibody administration.

In certain embodiments, the amount anti-HER3 antibody or antigen-binding fragment thereof is sufficient to achieve a Cₘᵢₙ of 50 μg/mL or greater antibody serum concentration. In other embodiments, the amount of the anti-HER3 antibody or antigen-binding fragment thereof is sufficient to achieve antibody serum concentration of 50 μg/mL or greater throughout the dosing interval (e.g., approximately 7 days, 14 days, 21 days, or 28 days). In one embodiment the dosing interval is 21 days. In certain embodiments, the cancer is a head and neck cancer. In certain embodiments, the cancer is a squamous cell carcinoma head and neck cancer. In particular embodiments, the cancer is a thyroid cancer. In certain embodiments, the cancer is resistant to treatment with a BRAF inhibitor (e.g., vemurafenib or dabrafenib). In another embodiment, the cancer is resistant to treatment with a MEK inhibitor (e.g., trametinib). In other embodiments, the cancer is resistant to treatment with a BRAF inhibitor (e.g., vemurafenib or dabrafenib) and a MEK inhibitor (e.g., trametinib). In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In one aspect, an amount of an anti-HER3 antibody (e.g., 2C2-YTE) or antigen-binding fragment thereof administered is sufficient to achieve an antibody serum concentration in the range of 50 μg/mL to 500 μg/mL, for example throughout a dosing interval, e.g., at least 1 week, 2 weeks, 3 weeks or 4 weeks between each administration. In a certain aspect, an amount of an anti-HER3 antibody (e.g., 2C2-YTE) or antigen-binding fragment thereof administered is sufficient to achieve an antibody serum concentration in the range of 50 μg/mL to 250 μg/mL, for example throughout a dosing interval, e.g., at least 1 week, 2 weeks, 3 weeks or 4 weeks between each administration. In one aspect, an amount of an anti-HER3 antibody (e.g., 2C2-YTE) or antigen-binding fragment thereof administered is sufficient to achieve an antibody serum concentration in the range of 50 μg/mL to 500 μg/mL, for example over a period of approximately 3 weeks between each administration. In a certain aspect, an amount of an anti-HER3 antibody (e.g., 2C2-YTE) or antigen-binding fragment thereof administered is sufficient to achieve an antibody serum concentration in the range of 50 μg/mL to 250 μg/mL, for example over a period of approximately 3 weeks between each administration. In one aspect, an amount of an anti-HER3 antibody (e.g., 2C2-YTE) or antigen-binding fragment thereof administered is sufficient to achieve an antibody serum concentration in the range of 50 μg/mL to 500 μg/mL, for example throughout a dosing interval, e.g., at least 6 days, at least 7 days, at least 8 days, at least 12 days, at least 13 days, at least 14 days, at least 15, days, at least 19 days, or at least 20 days, following antibody administration. In a certain aspect, an amount of an anti-HER3 antibody (e.g., 2C2-YTE) or antigen-binding fragment thereof administered is sufficient to achieve an antibody serum concentration in the range of 50 μg/mL to 250 μg/mL, for example throughout a dosing interval, e.g., at least 6 days, at least 7 days, at least 8 days, at least 12 days, at least 13 days, at least 14 days, at least 15, days, at least 19 days, or at least 20 days, following antibody administration.

In one aspect, an amount of an anti-HER3 antibody (e.g., 2C2-YTE) or antigen-binding fragment thereof administered is sufficient to achieve a Cₘᵢₙ antibody serum concentration in the range of 50 μg/mL to 500 μg/mL, for example over a period of at least 1 week, 2 weeks, 3 weeks or 4 weeks between each administration. In a certain aspect, an amount of an anti-HER3 antibody (e.g., 2C2-YTE) or antigen-binding fragment thereof administered is sufficient to achieve a Cₘᵢₙ antibody serum concentration in the range of 50 μg/mL to 250 μg/mL, for example over a period of at least 1 week, 2 weeks, 3 weeks or 4 weeks between each administration.

In one aspect, an amount of an anti-HER3 antibody (e.g., 2C2-YTE) or antigen-binding fragment thereof administered is sufficient to achieve a Cₘᵢₙ antibody serum concentration in the range of 50 μg/mL to 500 μg/mL, for example over a period of at least 6 days, at least 7 days, at least 8 days, at least 12 days, at least 13 days, at least 14 days, at least 15, days, at least 19 days, or at least 20 days, following antibody administration. In a certain aspect, an amount of an anti-HER3 antibody (e.g., 2C2-YTE) or antigen-binding fragment thereof administered is sufficient to achieve a Cₘᵢₙ antibody serum concentration in the range of 50 μg/mL to 250 μg/mL, for example over a period of at least 6 days, at least 7 days, at least 8 days, at least 12 days, at least 13 days, at least 14 days, at least 15, days, at least 19 days, or at least 20 days, following antibody administration.

In one aspect, an amount of an anti-HER3 antibody (e.g., 2C2-YTE) or antigen-binding fragment thereof administered is sufficient to achieve a Cₘᵢₙ antibody serum concentration in the range of 50 μg/mL to 500 μg/mL, for example over a period of approximately 3 weeks between each administration. In a certain aspect, an amount of an anti-HER3 antibody (e.g., 2C2-YTE) or antigen-binding fragment thereof administered is sufficient to achieve a Cₘᵢₙ antibody serum concentration in the range of 50 μg/mL to 250 μg/mL, for example over a period of approximately 3 weeks between each administration.

In a certain aspect, an amount of an anti-HER3 antibody (e.g., 2C2-YTE) or antigen-binding fragment thereof administered to the human subject is sufficient to achieve a Cₘᵢₙ of 50 μg/mL antibody serum concentration. In a specific aspect, an amount of an anti-HER3 antibody (e.g., 2C2-YTE) or antigen-binding fragment thereof administered to a human subject is sufficient to achieve (i) a Cₘᵢₙ of 50 μg/mL or greater antibody serum concentration, or (ii) an antibody serum concentration of 50 μg/mL or greater, over a period of approximately 3 weeks. In a specific aspect, an amount of an anti-HER3 antibody (e.g., 2C2-YTE) or antigen-binding fragment thereof administered is 5 mg/kg, 10 mg/kg, 15 mg/kg, or 20 mg/kg every week (7 days), every 2 weeks (14 days), every 3 weeks (21 days) or every 4 weeks (28 days). In one aspect, an amount of an anti-HER3 antibody (e.g., 2C2-YTE) or antigen-binding fragment thereof administered is 5 mg/kg every week or 10 mg/kg every two weeks (14 days). In a particular aspect, an amount of an anti-HER3 antibody (e.g., 2C2-YTE) or antigen-binding fragment thereof administered is 20 mg/kg every 3 weeks. In a specific aspect, an amount of an anti-HER3 antibody (e.g., 2C2-YTE) or antigen-binding fragment thereof administered is 5 mg/kg every week, every 2 weeks, every 3 weeks or every 4 weeks. In a specific aspect, an amount of an anti-HER3 antibody (e.g., 2C2-YTE) or antigen-binding fragment thereof administered is 10 mg/kg every week, every 2 weeks, every 3 weeks or every 4 weeks. In a specific aspect, an amount of an anti-HER3 antibody (e.g., 2C2-YTE) or antigen-binding fragment thereof administered is 15 mg/kg every week, every 2 weeks, every 3 weeks or every 4 weeks. In a specific aspect, an amount of an anti-HER3 antibody (e.g., 2C2-YTE) or antigen-binding fragment thereof administered is 20 mg/kg every week, every 2 weeks, every 3 weeks or every 4 weeks.

In one aspect, an amount of an anti-HER3 antibody (e.g., 2C2-YTE) or antigen-binding fragment thereof administered is a fixed dose of 1200 mg every week, every 2 weeks, every 3 weeks, or every 4 weeks. In a specific aspect, an amount of an anti-HER3 antibody (e.g., 2C2-YTE) or antigen-binding fragment thereof administered is 1200 mg every 3 weeks. In a specific aspect, an amount of an anti-HER3 antibody (e.g., 2C2-YTE) or antigen-binding fragment thereof administered is 1200 mg every 2 weeks. In a specific aspect, an amount of an anti-HER3 antibody (e.g., 2C2-YTE) or antigen-binding fragment thereof administered is 1200 mg every 4 weeks. In a particular aspect, an amount of an anti-HER3 antibody (e.g., 2C2-YTE) or antigen-binding fragment thereof administered is 5 mg/kg weekly or 500 mg weekly. In a certain aspect, an amount of the anti-HER3 antibody (e.g., 2C2-YTE) or antigen-binding fragment thereof administered is 10 mg/kg or 1000 mg every 2 weeks.

In a particular aspect, the human subject is administered a loading dose (e.g., a first or initial dose) of an anti-HER3 antibody (e.g., 2C2-YTE) or antigen-binding fragment thereof that is higher than a subsequent dose of the anti-HER3 antibody (e.g., 2C2-YTE) or antigen-binding fragment thereof. In one aspect, the loading dose (e.g., a first or initial dose) is at least 1.5 fold, at least 2 fold, at least 3 fold, at least 4 fold, or at least 5 fold higher than a subsequent dose of anti-HER3 (e.g., 2C2-YTE) or an antigen-binding fragment thereof. In one aspect, the loading dose (e.g., a first or initial dose) is at least 25%, 50%, or 75% higher than a subsequent dose of anti-HER3 (e.g., 2C2-YTE) or an antigen-binding fragment thereof. In a certain aspect, the loading dose (e.g., a first or initial dose) of an anti-HER3 antibody (e.g., 2C2-YTE) or an antigen-binding fragment thereof is administered 1 week, 2 weeks, 3 weeks, or 4 weeks prior to the subsequent dose of the anti-HER3 antibody or antigen-binding fragment thereof. In a certain aspect, the loading dose (e.g., a first or initial dose) is administered 1 week, 2 weeks, or 3 weeks prior to the subsequent dose of an anti-HER3 antibody or antigen-binding fragment thereof. In a particular aspect, the subsequent dose of an anti-HER3 antibody (e.g., 2C2-YTE) or antigen-binding fragment thereof is 5 mg/kg, 10 mg/kg, 15 mg/kg, or 20 mg/kg every week, every 2 weeks, every 3 weeks or every 4 weeks. In a particular aspect, the subsequent dose of an anti-HER3 antibody (e.g., 2C2-YTE) or antigen-binding fragment thereof is 5 mg/kg every week, 10 mg/kg every 2 weeks, 15 mg/kg every 3 weeks, or 20 mg/kg every 3 weeks.

In specific aspects, disclosed herein is a method of treating an HPV positive SCCHN in a human subject in need thereof, comprising administering to the human subject a therapeutically effective amount of an anti-HER3 antibody (e.g., 2C2-YTE) or antigen-binding fragment thereof which specifically binds to an epitope within the extracellular domain of HER3, wherein the anti-HER3 antibody specifically binds to the same HER3 epitope as an antibody or antigen-binding fragment thereof comprising the heavy chain variable region (VH) and light chain variable region (VL) of CL16 or 2C2 or competitively inhibits HER3 binding by an antibody or antigen-binding fragment thereof comprising the VH and VL of CL16 or 2C2. In a particular aspect, the VH and VL of CL16 comprise SEQ ID NOs: 2 and 1, respectively, and the VH and VL of 2C2 comprise SEQ ID NOs: 2 and 3, respectively. In a specific aspect, disclosed herein is a method of treating an HPV positive SCCHN in a human subject in need thereof, comprising administering to the human subject a therapeutically effective amount of an anti-HER3 antibody (e.g., 2C2-YTE). In one aspect, the amount of the anti-HER3 antibody or antigen-binding fragment thereof administered is a fixed dose of 1000 mg every week, every 2 weeks, every 3 weeks, or every 4 weeks. In a specific aspect, the amount of the anti-HER3 antibody or antigen-binding fragment thereof administered is 1000 mg every 2 weeks. In one aspect, the amount of the anti-HER3 antibody or antigen-binding fragment thereof administered is sufficient to achieve an antibody serum concentration in the range of 50 μg/mL to 500 μg/mL, for example over a period of 1 week, 2 weeks, 3 weeks or 4 weeks between each administration. In a certain aspect, the anti-HER3 antibody or antigen-binding fragment thereof is administered intravenously. In one aspect, the anti-HER3 antibody or antigen-binding fragment thereof is administered as a 60-minute intravenous infusion. In a specific aspect, a method of treating HPV positive SCCHN described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV positive SCCHN. In a certain aspect, the SCCHN is EGFR expressing SCCHN. In a particular aspect, the human subject is administered (i) the anti-HER3 antibody or antigen-binding fragment (e.g., at a dose sufficient to achieve a target serum concentration, such as a Cₘᵢₙ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) cetuximab at an initial loading dose of 400 mg/m² on day 2 over approximately 2 hours followed by weekly doses of 250 mg/m² over approximately 60 minutes starting on day 8 and continuing weekly. In one embodiment, the dose of the anti-HER3 antibody or antigen-binding fragment thereof is 15 mg/kg. In a particular aspect, the human subject is administered (i) the anti-HER3 antibody or antigen-binding fragment (e.g., at a dose sufficient to achieve a target serum concentration, such as a Cₘ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) cetuximab on day 1 at a dose of 400 mg/m² or 250 mg/m² weekly or at an initial loading dose of 400 mg/m² followed by a weekly dose of 250 mg/m². In one embodiment, the dose of the anti-HER3 antibody or antigen-binding fragment thereof is 15 mg/kg. In a certain aspect, the anti-HER3 antibody or antigen-binding fragment and the cetuximab are administered approximately 2 hours apart on the same day. In certain embodiments, the HPV positive head and neck cancer (e.g. SCCHN) is a cancer of the oral cavity, hypopharynx, oropharynx, rynopharynx, or larynx. In certain embodiments, the HPV positive SCCHN is an oropharyngeal cancer.

In specific aspects, disclosed herein is a method of treating an HPV negative SCCHN in a human subject in need thereof, comprising administering to the human subject a therapeutically effective amount of an anti-HER3 antibody (e.g., 2C2-YTE) or antigen-binding fragment thereof which specifically binds to an epitope within the extracellular domain of HER3, wherein the anti-HER3 antibody specifically binds to the same HER3 epitope as an antibody or antigen-binding fragment thereof comprising the heavy chain variable region (VH) and light chain variable region (VL) of CL16 or 2C2 or competitively inhibits HER3 binding by an antibody or antigen-binding fragment thereof comprising the VH and VL of CL16 or 2C2. In a particular aspect, the VH and VL of CL16 comprise SEQ ID NOs: 2 and 1, respectively, and the VH and VL of 2C2 comprise SEQ ID NOs: 2 and 3, respectively. In a specific aspect, disclosed herein is a method of treating an HPV negative SCCHN in a human subject in need thereof, comprising administering to the human subject a therapeutically effective amount of an anti-HER3 antibody (e.g., 2C2-YTE). In one aspect, the amount of the anti-HER3 antibody or antigen-binding fragment thereof administered is a fixed dose of 1000 mg every week, every 2 weeks, every 3 weeks, or every 4 weeks. In a specific aspect, the amount of the anti-HER3 antibody or antigen-binding fragment thereof administered is 1000 mg every 2 weeks. In one aspect, the amount of the anti-HER3 antibody or antigen-binding fragment thereof administered is sufficient to achieve an antibody serum concentration in the range of 50 μg/mL to 500 μg/mL, for example over a period of 1 week, 2 weeks, 3 weeks or 4 weeks between each administration. In a certain aspect, the anti-HER3 antibody or antigen-binding fragment thereof is administered intravenously. In one aspect, the anti-HER3 antibody or antigen-binding fragment thereof is administered as a 60-minute intravenous infusion. In a specific aspect, a method of treating HPV negative SCCHN described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody such as cetuximab. In a particular aspect, the human subject has been diagnosed with an HPV negative SCCHN. In a certain aspect, the SCCHN is EGFR expressing SCCHN. In a particular aspect, the human subject is administered (i) the anti-HER3 antibody or antigen-binding fragment (e.g., at a dose sufficient to achieve a target serum concentration, such as a Cₘ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) cetuximab at an initial loading dose of 400 mg/m² on day 2 over approximately 2 hours followed by weekly doses of 250 mg/m² over approximately 60 minutes starting on day 8 and continuing weekly. In one embodiment, the dose of the anti-HER3 antibody or antigen-binding fragment thereof is 15 mg/kg. In a particular aspect, the human subject is administered (i) the anti-HER3 antibody or antigen-binding fragment (e.g., at a dose sufficient to achieve a target serum concentration, such as a Cₘ of 50 μg/mL or greater antibody serum concentration, or antibody serum concentration of 50 μg/mL or greater, throughout the dosing interval, or at a dose of 15 mg/kg or 20 mg/kg) by intravenous (IV) infusion on day 1 and repeated every 21 days; and (ii) cetuximab on day 1 at a dose of 400 mg/m² or 250 mg/m² weekly or at an initial loading dose of 400 mg/m² followed by a weekly dose of 250 mg/m². In one embodiment, the dose of the anti-HER3 antibody or antigen-binding fragment thereof is 15 mg/kg. In a certain aspect, the anti-HER3 antibody or antigen-binding fragment and the cetuximab are administered approximately 2 hours apart on the same day. In certain embodiments, the HPV positive head and neck cancer (e.g. SCCHN) is a cancer of the oral cavity, hypopharynx, oropharynx, rynopharynx, or larynx.

A cancer is considered an HPV positive cancer when evidence of HPV or a marker indicating the presence of HPV (e.g., HPV DNA or p16) is detected in the cancer or cells of the cancer. The HPV status (i.e., positive or negative) of a head and neck cancer (e.g., SCCHN) from a human subject may be determined, for example, using various methods, such as but not limited to type-specific polymerase chain reaction (PCR), real-time PCR (RT-PCR), immunohistochemical detection of surrogate markers such as p16, HPV deoxyribonucleic acid (DNA) in situ hybridization (ISH), southern blot hybridization (SBH), dot blot hybridization, or a hybrid capture-2 assay (see, e.g., Smith et al., 2014, Oral Oncol. 50(6):600-604). The specimen type for determination of HPV status using such methods may be, for example, a biopsy, a scrape, a brushing, or a mouth rinse. The specimen may be stored, for example, as a fresh frozen (FF) or formalin fixed paraffin-embedded (PE) biopsy.

In a particular aspect of a method of treating cancer described herein with an anti-HER3 antibody (e.g., 2C2-YTE) or an antigen-binding fragment thereof, the cancer is selected from the group consisting of melanoma, thyroid cancer, pancreatic cancer, colon or colorectal cancer, lung cancer (e.g., non small cell lung cancer), gastric cancer, breast cancer, and head and neck cancer (e.g., squamous cell carcinoma of the head and neck). In particular aspects, the cancer comprises cancer cells expressing HERS. In a particular aspect, the cancer comprises cancer cells expressing heregulin-1. In a particular aspect, the cancer comprises cells comprising a KRAS mutation. In certain aspect, the KRAS mutation comprises a mutation of codon 12 of a human KRAS gene. In one aspect, the cancer is KRAS mutation negative. In a particular aspect, the cancer comprises cells comprising a BRAF mutation (e.g., V600E or V600K). In particular aspects, the cancer comprises cancer cells expressing HER2. In particular aspects, the cancer comprises cancer cells expressing EGFR.

In one aspect, a method of treating cancer described herein with an anti-HER3 antibody (e.g., 2C2-YTE) or an antigen-binding fragment thereof further comprises administering to the human subject a therapeutic effective amount of a BRAF inhibitor. In one aspect, the BRAF inhibitor is vemurafenib. In one aspect, the BRAF inhibitor is dabrafenib. In a specific aspect, the cancer comprises cells comprising a BRAF mutation. In a particular aspect, the BRAF mutation is the BRAF V600E mutation. In a particular aspect, the BRAF mutation is the BRAF V600K mutation. In a certain aspect, the BRAF inhibitor is an antibody or antigen binding fragment thereof. In a particular aspect, the human subject has been diagnosed with melanoma or BRAF mutated melanoma. In a certain aspect, the human subject is administered (i) an anti-HER3 antibody (e.g., 2C2-YTE) or antigen-binding fragment by intravenous (IV) infusion on day 1 and repeated every 21 days; and (ii) vemurafenib at a dose of 960 mg twice daily, e.g., approximately every 12 hours, starting on day 2. In certain embodiments, the cancer is a head and neck cancer. In certain embodiments, the cancer is a squamous cell carcinoma head and neck cancer. In particular embodiments, the cancer is a thyroid cancer. In certain embodiments, prior to administration of the HER3 antibody, the cancer is resistant to treatment with the BRAF inhibitor (e.g., vemurafenib or dabrafenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer (e.g., melanoma) described herein with an anti-HER3 antibody (e.g., 2C2-YTE) or an antigen-binding fragment thereof further comprises administering to the subject a therapeutic effective amount of a MEK inhibitor. In one aspect the MEK inhibitor is trametinib. In certain embodiments, the cancer is a head and neck cancer. In certain embodiments, the cancer is a squamous cell carcinoma head and neck cancer. In particular embodiments, the cancer is a thyroid cancer. In certain embodiments, prior to administration of the HER3 antibody, the cancer is resistant to treatment with the MEK inhibitor (e.g., trametenib). In a particular embodiment, prior to administration of the HER3 antibody, the cancer is resistant to treatment with trametinib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer described herein with an anti-HER3 antibody (e.g., 2C2-YTE) or an antigen-binding fragment thereof further comprises administering to the subject a therapeutic effective amount of a MEK inhibitor and a BRAF inhibitor. In certain embodiments, prior to administration of the HER3 antibody, the cancer is resistant to treatment with the BRAF inhibitor (e.g., vemurafenib or dabrafenib) and the MEK inhibitor (e.g., trametenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In a particular embodiment, prior to administration of the HER3 antibody, the cancer is resistant to treatment with trametinib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer (e.g., colon or colorectal cancer or lung cancer) described herein with an anti-HER3 antibody (e.g., 2C2-YTE) or an antigen-binding fragment thereof further comprises administering to the human subject a therapeutically effective amount of an EGFR inhibitor. In one aspect, the EGFR inhibitor is an anti-EGFR antibody, a reversible small molecule inhibitor of EGFR (e.g., erlotinib), or an irreversible small molecule inhibitor of EGFR (e.g., AZD9291). In one aspect, the EGFR inhibitor is cetuximab. In a particular aspect, the human subject has been diagnosed with head and neck cancer, such as squamous cell carcinoma of the head and neck. In a certain aspect, the head and neck cancer is EGFR expressing head and neck cancer. In one aspect, the human subject has been diagnosed with colon or colorectal cancer. In a certain aspect, the human subject has been diagnosed with EGFR expressing colon or colorectal cancer. In one aspect, the human subject has been diagnosed with KRAS mutated negative, EGFR expressing colon cancer. In a particular aspect, the human subject is administered (i) an anti-HER3 antibody (e.g., 2C2-YTE) or antigen-binding fragment by intravenous (IV) infusion on day 1 and repeated every 21 days; and (ii) cetuximab at an initial loading dose of 400 mg/m² on day 2 over approximately 2 hours followed by weekly doses of 250 mg/m² over approximately 60 minutes starting on day 8 and continuing weekly. In a particular aspect, the human subject is administered (i) the anti-HER3 antibody (e.g., 2C2-YTE) or antigen-binding fragment by intravenous (IV) infusion on day 1 and repeated every 21 days; and (ii) cetuximab on day 1 at a dose of 400 mg/m² or 250 mg/m² weekly or at an initial loading dose of 400 mg/m² followed by a weekly dose of 250 mg/m². In a certain aspect, the anti-HER3 antibody (e.g., 2C2-YTE) or antigen-binding fragment and the cetuximab are administered approximately 2 hours apart on the same day. In certain embodiments, prior to administration of the HER3 antibody, the cancer is resistant to treatment with an EGFR inhibitor (e.g., cetuximab or trastuzumab). In a particular embodiment, prior to administration of the HER3 antibody, the cancer is resistant to treatment with cetuximab. In another particular embodiment, prior to administration of the HER3 antibody, the cancer is resistant to treatment with trastuzumab. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer (e.g., lung cancer such as non small cell lung cancer) described herein with an anti-HER3 antibody (e.g., 2C2-YTE) or an antigen-binding fragment thereof further comprises administering to the human subject a therapeutically effective amount of a small molecule inhibitor (e.g., reversible or irreversible small molecule inhibitor) of EGFR such as erlotinib. In a particular aspect, the human subject has been diagnosed with lung cancer, such as non-small cell lung cancer. In certain embodiments, the cancer is a head and neck cancer. In certain embodiments, the cancer is a squamous cell carcinoma head and neck cancer. In particular embodiments, the cancer is a thyroid cancer. In a certain aspect, the human subject is administered (i) an anti-HER3 antibody (e.g., 2C2-YTE) or antigen-binding fragment; and (ii) erlotinib at a dose of 150 mg/day orally or 100 mg/day orally. In a particular aspect, the human subject has been diagnosed with lung cancer, such as non-small cell lung cancer. In certain embodiments, the cancer is a head and neck cancer. In certain embodiments, the cancer is a squamous cell carcinoma head and neck cancer. In particular embodiments, the cancer is a thyroid cancer. In a certain aspect, the human subject is administered (i) an anti-HER3 antibody (e.g., 2C2-YTE) or antigen-binding fragment by intravenous (IV) infusion on day 1 and repeated every 21 days; and (ii) erlotinib on day 2 at a dose of 150 mg/day orally. In a particular aspect, the human subject is administered (i) the anti-HER3 antibody (e.g., 2C2-YTE) or antigen-binding fragment by intravenous (IV) infusion on day 1 and repeated every 21 days; and (ii) erlotinib on day 1 at a dose of 150 mg/day orally. In a particular aspect, the human subject has been diagnosed with lung cancer, such as non-small cell lung cancer. In certain embodiments, the cancer is a head and neck cancer. In certain embodiments, the cancer is a squamous cell carcinoma head and neck cancer. In particular embodiments, the cancer is a thyroid cancer. In a certain aspect, the human subject is administered (i) an anti-HER3 antibody (e.g., 2C2-YTE) or antigen-binding fragment by intravenous (IV) infusion on day 1 and repeated every 21 days; and (ii) erlotinib on day 2 at a dose of 100 mg/day orally. In a particular aspect, the human subject is administered (i) the anti-HER3 antibody (e.g., 2C2-YTE) or antigen-binding fragment by intravenous (IV) infusion on day 1 and repeated every 21 days; and (ii) erlotinib on day 1 at a dose of 100 mg/day orally. In certain embodiments, the cancer is resistant to treatment with the EGFR inhibitor (e.g., erlotinib or AZD9291). In a particular embodiment, prior to administration of the HER3 antibody, the cancer is resistant to treatment with erlotinib. In certain embodiments, the cancer comprises cells that overexpress a HER3 ligand, for example, neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer (e.g., breast cancer or gastric cancer) described herein with an anti-HER3 antibody (e.g., 2C2-YTE) or an antigen-binding fragment thereof further comprises administering to the human subject a therapeutically effective amount of a HER2 inhibitor (e.g., anti-HER2 antibody or small molecule inhibitor of HER2). In a particular aspect, the HER2 inhibitor is an anti-HER2 antibody such as trastuzumab. In a certain aspect, the human subject has been diagnosed with breast cancer, for example HER2 positive breast cancer. In one aspect, the human subject has been diagnosed with gastric cancer, for example, HER2 positive gastric cancer. In a particular aspect, the human subject had failed initial therapy for advanced or metastatic disease. In a specific aspect, the human subject is administered (i) the anti-HER3 antibody (e.g., 2C2-YTE) or antigen-binding fragment by intravenous (IV) infusion on day 1 and repeated every 21 days; and (ii) trastuzumab at an initial loading dose of 4 mg/kg IV on day 2 over approximately 90 minutes followed by 2 mg/kg IV over approximately 30 minutes starting on day 8 and continuing weekly. In a certain aspect, the human subject is administered (i) an anti-HER3 antibody (e.g., 2C2-YTE) or antigen-binding fragment by intravenous (IV) infusion on day 1 and repeated every 21 days; and (ii) trastuzumab at a dose of 4 mg/kg or 2 mg/kg IV weekly. In one aspect, the anti-HER3 antibody (e.g., 2C2-YTE) or antigen-binding fragment and trastuzumab are administered approximately 2 hours apart on the same day. In a particular aspect, the human subject is administered (i) the anti-HER3 antibody (e.g., 2C2-YTE) or antigen-binding fragment by intravenous (IV) infusion on day 1 and repeated every 21 days; and (ii) trastuzumab at an initial loading dose of 8 mg/kg IV on day 2 over approximately 90 minutes followed by 6 mg/kg IV over approximately 30 minutes starting on day 8 and continuing every 3 weeks. In one aspect, the human subject is administered (i) the anti-HER3 antibody (e.g., 2C2-YTE) or antigen-binding fragment by intravenous (IV) infusion on day 1 and repeated every 21 days; and (ii) trastuzumab at a dose of 8 mg/kg or 6 mg/kg IV every 3 weeks. In certain embodiments, prior to administration of the HER3 antibody, the cancer is resistant to treatment with an EGFR inhibitor (e.g., cetuximab or trastuzumab). In a particular embodiment, prior to administration of the HER3 antibody, the cancer is resistant to treatment with cetuximab. In another particular embodiment, prior to administration of the HER3 antibody, the cancer is resistant to treatment with trastuzumab. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer described herein is a method of treating thyroid cancer. In one embodiment, described herein is a method for treating thyroid cancer in a human subject, wherein the human subject has been diagnosed with thyroid cancer. In a particular embodiment, described herein is a method of treating BRAF mutated thyroid cancer. In a more particular embodiment, described herein is a method of treating V600 BRAF mutated thyroid cancer. In another more particular embodiment, described herein is a method of treating V600E BRAF mutated thyroid cancer. In another particular embodiment, described herein is a method of treating radioiodine refractory (RAIR) thyroid cancer. In a more particular embodiment, described herein is a method of treating a BRAF mutated RAIR thyroid cancer. In another more particular embodiment, described herein is a method of treating a V600 BRAF mutated RAIR thyroid cancer. In yet another more particular embodiment, described herein is a method of treating a V600E BRAF mutated RAIR thyroid cancer. In a specific embodiment, described herein is a method of treating thyroid cancer wherein the thyroid cancer comprises a differentiated thyroid carcinoma. In another specific embodiment, described herein is a method of treating thyroid cancer wherein the thyroid cancer comprises a differentiated thyroid carcinoma of follicular origin. In yet another specific embodiment, described herein is a method of treating thyroid cancer wherein the thyroid cancer comprises a differentiated thyroid carcinoma of papillary origin. In another more specific embodiment, described herein is a method of treating thyroid cancer wherein the differentiated thyroid carcinoma of papillary origin comprises a Hurthle cell carcinoma. In a specific embodiment, described herein is a method of treating thyroid cancer wherein the thyroid cancer is a papillary thyroid cancer. In another specific embodiment, described herein is a method of treating thyroid cancer wherein the thyroid cancer is a follicular thyroid cancer. In yet another embodiment, described herein is a method of treating thyroid cancer wherein the thyroid cancer is a medullary thyroid cancer. In yet another embodiment, described herein is a method of treating thyroid cancer wherein the thyroid cancer is anaplastic thyroid cancer. In particular embodiments, described herein is a method of treating thyroid cancer, wherein the thyroid cancer comprises a metastatic thyroid cancer.

In one embodiment, described herein is a method for treating thyroid cancer in a human subject, wherein the human subject has been diagnosed with thyroid cancer, for example, BRAF mutated thyroid cancer or RAIR thyroid cancer, e.g., BRAF mutated RAIR thyroid cancer. In a specific embodiment, the RAIR thyroid cancer is a papillary or follicular thyroid cancer. In a particular embodiment, the method comprises administering to the human subject an anti-HER3 antibody (e.g., 2C2-YTE) or an antigen-binding fragment, for example, at a fixed dose of 500 to 1500 mg, e.g., a fixed dose of 500 mg, 600 mg, 700 mg, 800 mg, 900 mg, 1000 mg, 1100 mg, 1200 mg, 1300 mg, 1400 mg, or 1500 mg and a BRAF inhibitor, e.g., vemurafenib, e.g., 960 mg vemurafenib twice daily, e.g., every 12 hours. In a particular embodiment, the method comprises administering to the human subject an anti-HER3 antibody (e.g., 2C2-YTE) or an antigen-binding fragment at a fixed dose of 500 to 1500 mg. In a more particular embodiment, the method comprises administering to the human subject an anti-HER3 antibody (e.g., 2C2-YTE) or an antigen-binding fragment at a fixed dose of 500 mg. In another more particular embodiment, the method comprises administering to the human subject an anti-HER3 antibody (e.g., 2C2-YTE) or an antigen-binding fragment at a fixed dose of 600 mg. In another more particular embodiment, the method comprises administering to the human subject an anti-HER3 antibody (e.g., 2C2-YTE) or an antigen-binding fragment at a fixed dose of 700 mg. In another more particular embodiment, the method comprises administering to the human subject an anti-HER3 antibody (e.g., 2C2-YTE) or an antigen-binding fragment at a fixed dose of 800 mg. In another more particular embodiment, the method comprises administering to the human subject an anti-HER3 antibody (e.g., 2C2-YTE) or an antigen-binding fragment at a fixed dose of 900 mg. In another more particular embodiment, the method comprises administering to the human subject an anti-HER3 antibody (e.g., 2C2-YTE) or an antigen-binding fragment at a fixed dose of 1000 mg. In another more particular embodiment, the method comprises administering to the human subject an anti-HER3 antibody (e.g., 2C2-YTE) or an antigen-binding fragment at a fixed dose of 1100 mg. In another more particular embodiment, the method comprises administering to the human subject an anti-HER3 antibody (e.g., 2C2-YTE) or an antigen-binding fragment at a fixed dose of 1200 mg. In another more particular embodiment, the method comprises administering to the human subject an anti-HER3 antibody (e.g., 2C2-YTE) or an antigen-binding fragment at a fixed dose of 1300 mg. In another more particular embodiment, the method comprises administering to the human subject an anti-HER3 antibody (e.g., 2C2-YTE) or an antigen-binding fragment at a fixed dose of 1400 mg. In another more particular embodiment, the method comprises administering to the human subject an anti-HER3 antibody (e.g., 2C2-YTE) or an antigen-binding fragment at a fixed dose of 1500 mg. In one embodiment, the method comprises administering to the human subject a 2C2-YTE anti-HER3 antibody. In one embodiment, the method comprises administering to the human subject a BRAF inhibitor, wherein the BRAF inhibitor is vemurafenib. In one embodiment, the method comprises administering to the human subject a BRAF inhibitor, wherein the BRAF inhibitor is dabrafenib. In a particular embodiment, the method comprises administering to the human subject 960 mg vemurafenib twice daily, e.g., every 12 hours. In yet another embodiment, the method comprises administering to the human subject a 2C2-YTE antibody and vemurafenib. In one embodiment, the anti-HER3 antibody or antigen-binding fragment thereof is administered every 3 weeks, or 21 days. In certain embodiments, prior to administration of the HER3 antibody, the cancer is resistant to treatment with the BRAF inhibitor (e.g., vemurafenib or dabrafenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In another embodiment, a method for treating thyroid cancer described herein further comprises administering to the subject radioactive iodine, e.g., ¹³¹I or ¹²⁵I, for example, with thyroid stimulating hormone (TSH), e.g., recombinant human TSH, for example thyrotropin alpha (thyrogen), stimulation. In a particular embodiment, a method for treating thyroid cancer described herein further comprises administering to the subject ¹³¹I. In another particular embodiment, a method for treating thyroid cancer described herein further comprises administering to the subject ¹³¹I with TSH stimulation. In a more particular embodiment, the TSH is recombinant human TSH. In another more particular embodiment, the TSH is thyrotropin alpha (thyrogen). In a specific embodiment, the radioiodine administration is performed after the anti-HER3 antibody (e.g., 2C2-YTE) and BRAF inhibitor administration, e.g., 5, 6, 7, 8, 9, or 10 days after said administration of the anti-HER3 antibody (e.g., 2C2-YTE) or antigen-binding fragment, and/or after 1, 2, 3, 4, 5 or 6 doses of the anti-HER3 antibody (e.g., 2C2-YTE) or antigen-binding fragment thereof. In a particular embodiment, the human subject is administered a 2C2-YTE antibody. In a certain embodiments, administration of the BRAF inhibitor, e.g., vemurafenib, continues after radioiodine administration, for example, continues 2, 3, 4, 5, 6, or 7 days after such administration. In a particular embodiment, the BRAF inhibitor is vemurafenib. In a particular embodiment, the method comprises administering to the human subject 2C2-YTE and vemurafenib. In another particular embodiment, the method comprises administering to the human subject 2C2-YTE and vemurafenib, and further comprises administering to the subject with ¹³¹I, for example, with TSH stimulation. In yet another particular embodiment, the method comprises administering to the human subject 2C2-YTE and vemurafenib, and further comprises administering to the subject with ¹³¹I with TSH stimulation. In one embodiment, the anti-HER3 antibody or antigen-binding fragment thereof is administered every 3 weeks, or 21 days. In certain embodiments, prior to administration of the HER3 antibody, the cancer is resistant to treatment with the BRAF inhibitor (e.g., vemurafenib or dabrafenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a particular embodiment of a method for treating thyroid cancer, the human subject is administered (i) vemurafenib, for example at a dose of 500 mg, 600 mg, 700 mg, 800 mg, 900 mg, 960 mg, 1000 mg, 1100 mg, 1200 mg, or 1300 mg twice daily, e.g., approximately every 12 hours, starting on day 1; and (ii) an anti-HER3 antibody (e.g., 2C2-YTE) or antigen-binding fragment, for example at a fixed dose of 500 to 1500 mg, e.g., a fixed dose of 500 mg, 600 mg, 700 mg, 800 mg, 900 mg, 1000 mg, 1100 mg, 1200 mg, 1300 mg, 1400 mg, or 1500 mg, for example via intravenous (IV) infusion, on day 5, 6, 7, 8, 9, 10, 11, 12, 13, or 14, and repeated every 10, 11, 12, 13, 14, 15, 16, 17, 18, 19, 20, or 21 days. In certain embodiments, administration of the anti-HER3 antibody (e.g., 2C2-YTE) or antigen-binding fragment, is repeated 0, 1, 2, 3, 4, or 5 times. In another particular embodiment, administration further comprises (iii) administering to the human subject radioiodine, e.g., ¹³¹I, for example, in oral solution at a dose of 5 mCi to 300 mCi (e.g., 100 mCi to 150 mCi), optionally with TSH, e.g., recombinant human TSH, for example thyrogen, stimulation, after (for example, the week after, e.g., 5, 6, 7, 8, 9 or 10 days after) administration of the anti-HER 2 antibody (e.g., 2C2-YTE) or an antigen-binding fragment thereof, for example after the first, second, third or fourth dose the anti-HER3 antibody (e.g., 2C2-YTE) or an antigen-binding fragment thereof. In certain embodiments, one or more additional doses of radioiodine, e.g., ¹³¹I, for example, in oral solution at a dose of 5 mCi to 300 mCi (e.g., 100 mCi to 150 mCi), optionally with TSH, e.g., recombinant human TSH, for example thyrogen, stimulation, is administered after the initial dose of radioiodine, e.g., at standard treatment intervals for the administration of radioiodine. In certain embodiments, radioiodine e.g., ¹³¹I, for example, in oral solution at a dose of 5 mCi to 300 mCi (e.g., 50 mCi), is administered such that there is partial or complete ablation of normal thyroid tissue.

In a specific embodiment, TSH-stimulated radioiodine lesional dosimetry, e.g., thyrogen-stimulated ¹²⁴I PET/CT lesional dosimetry, is performed prior to administration of the BRAF inhibitor, e.g., vemurafenib, begins. In another specific embodiment, TSH-stimulated radioiodine lesional dosimetry, e.g., thyrogen-stimulated ¹²⁴I PET/CT lesional dosimetry, is performed after administration of the BRAF inhibitor, e.g., vemurafenib, begins, for example, after the second or third dose of anti-HER3 antibody or antigen-binding fragment.

In a particular embodiment, treatment is continued if the human subject demonstrates that a sufficient amount of radioiodine can be delivered to at least one tumor. For example, in a specific embodiment, treatment is continued if the human subject demonstrates that ≧2,000 cGy can be delivered to at least one tumor with <300 mCi of ¹³¹I (e.g., as determined by TSH, e.g., thyrogen-stimulated radioiodine lesional dosimetry, e.g., ¹²⁴I PET/CT lesional dosimetry)

In particular aspects, disclosed herein is a method of treating thyroid cancer (e.g., BRAF mutated thyroid cancer, or radioiodine refractory (RAIR) thyroid cancer, for example, BRAF-mutated RAIR thyroid cancer) in a human subject in need thereof, comprising administering to the subject an anti-HER3 antibody (e.g., 2C2-YTE antibody) or antigen-binding fragment and a MEK inhibitor (e.g., selumetinib or trametinib) and/or a BRAF inhibitor (e.g., dabrafenib or vemurafenib). In a specific embodiment, the RAIR thyroid cancer is a papillary or follicular thyroid cancer. In a specific embodiment, such a method comprises administering to the subject an anti-HER3 antibody (e.g., 2C2-YTE antibody) or antigen-binding fragment, a MEK inhibitor (e.g., selumetinib or trametinib) and a BRAF inhibitor (e.g., dabrafenib or vemurafenib). In a more specific embodiment, such methods can further comprise administering radioactive iodine, e.g., ¹³¹I or ¹²⁵I, for example, administering the radioactive iodine after administration of any or all of the anti-HER3 antibody or antigen binding fragment thereof, MEK inhibitor or BRAF inhibitor administration has begun. In a particular embodiment, the human subject has radioiodine refractory disease. In one embodiment, the dose of the anti-HER3 antibody or antigen-binding fragment thereof is 15 mg/kg. In one embodiment, the anti-HER3 antibody or antigen-binding fragment thereof is administered every 3 weeks, or 21 days. In certain embodiments, prior to administration of the HER3 antibody, the cancer is resistant to treatment with the BRAF inhibitor (e.g., vemurafenib or dabrafenib) and the MEK inhibitor (e.g., trametenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In a particular embodiment, prior to administration of the HER3 antibody, the cancer is resistant to treatment with trametinib. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In a specific aspect, a method of treating cancer described herein with an anti-HER3 antibody (e.g., 2C2-YTE) or an antigen-binding fragment thereof, as a monotherapy or a combination therapy, further comprises measuring the expression level (e.g., concentration) of a pharmacodynamic marker in a sample from the human subject. In certain aspects, the pharmacodynamic marker is soluble HER3, TWEAK, TWEAK receptor, Neurexophilin-1, IL-18 binding protein, ANGL4, IL-18 receptor 1, or Kallikrein 7. In a certain aspect, the pharmacodynamic marker is measured prior to, and after, administration of one or more doses of an anti-HER3 antibody (e.g., 2C2-YTE) or antigen-binding fragment. In a certain aspect, the pharmacodynamic marker is measured prior to, and after, administration of the anti-HER3 antibody (e.g., 2C2-YTE) or antigen-binding fragment. In a particular aspect, the circulating concentration of a pharmacodynamic marker is measured by methods known in the art, for example, by ELISA or proteomic aptamer based approaches (e.g., aptamer-based multiplexed proteomic technology, see, e.g., Gold et al., 2010, PLOS ONE, 5:e15004). In a certain aspect, a pharmacodynamic marker is measured prior to, and after (e.g., 1 week, 2 weeks, 3 weeks, or 4 weeks), administration of an anti-HER3 antibody (e.g., 2C2-YTE) or antigen-binding fragment. In a certain aspect, a pharmacodynamic marker is measured prior to, and after (e.g., 1 week, 2 weeks, 3 weeks, or 4 weeks), administration of a first dose of an anti-HER3 antibody (e.g., 2C2-YTE) or antigen-binding fragment. In one aspect, the circulating concentration (e.g., circulating concentration in plasma) of a pharmacodynamic marker is measured, for example by ELISA. In specific aspects, changes (e.g., increase or decrease) in the concentration (e.g., circulating concentration in plasma) of a pharmacodynamic marker, such as soluble HER3, TWEAK, TWEAK receptor, Neurexophilin-1, IL-18 binding protein, ANGL4, IL-18 receptor 1, or Kallikrein 7, is indicative of treatment. In a particular aspect, an increase in soluble HER3 circulating concentration detected for example by ELISA, following treatment with the anti-HER3 antibody (e.g., 2C2-YTE) or antigen-binding fragment thereof is indicative of treatment. In a particular aspect, a decrease in soluble HER3 circulating concentration detected for example by proteomic aptamer based approaches (e.g., aptamer-based multiplexed proteomic technology, see, e.g., Gold et al., 2010, PLOS ONE, 5:e15004), following treatment with an anti-HER3 antibody (e.g., 2C2-YTE) or antigen-binding fragment thereof is indicative of treatment. In a certain aspect, an increase in circulating concentration of TWEAK or TWEAK receptor following treatment with the anti-HER3 antibody (e.g., 2C2-YTE) or antigen-binding fragment thereof is indicative of treatment. In a particular aspect, a decrease in circulating concentration of Neurexophilin-1, IL-18 binding protein, IL-18 receptor 1, ANGL4, or Kallinkrein 7 following treatment with the anti-HER3 antibody (e.g., 2C2-YTE) or antigen-binding fragment thereof is indicative of treatment.

In specific aspects, provided herein are methods for treating cancer (e.g., head and neck cancer, breast cancer, colon or colorectal cancer, pancreatic cancer, thyroid cancer, lung cancer (e.g., non-small cell lung cancer), gastric cancer, ovarian cancer, or melanoma) comprising administering to a human subject in need thereof a BRAF inhibitor (e.g., dabrafenib or vemurafenib) and an anti-HER3 antibody or antigen binding fragment thereof described herein (e.g., antibody 2C2 or 2C2-YTE).

In specific aspects, the agent for use in combination with anti-HER3 binding molecules, e.g., antibodies or antigen-binding fragments, variants, or derivatives thereof described herein, is cetuximab (ERBITUX®), erlotinib)(TARCEVA®, trastuzumab (HERCEPTIN®), or vemurafenib (ZELBORAF®).

Provided herein are methods of treating cancer (e.g., head and neck cancer, colon cancer, non-small cell lung cancer, melanoma, breast cancer, or gastric cancer) in a subject comprising administering to the subject an anti-HER3 antibody (e.g., 2C2-YTE) or an antigen-binding fragment thereof at a dose of 15 mg/kg or 20 mg/kg, in combination with a BRAF inhibitor (e.g., dabrafenib or vemurafenib), an EGFR inhibitor (e.g., cetuximab or erlotinib) or a HER2 inhibitor (e.g., trastuzumab). In one embodiment, the dose of the anti-HER3 antibody or antigen-binding fragment thereof is 15 mg/kg.

In particular aspects, provided herein are methods for treating head and neck cancer or colon cancer (e.g., KRAS mutated, EGFR expressing colon cancer) in a human subject comprising administering to the subject an anti-HER3 antibody (e.g., 2C2-YTE) or an antigen-binding fragment thereof at a dose of 15 mg/kg or 20 mg/kg every 21 days, in combination with an EGFR inhibitor such as cetuximab. In one embodiment, the dose of the anti-HER3 antibody or antigen-binding fragment thereof is 15 mg/kg.

Provided herein are doses of cetuximab that are useful for combination therapy with an anti-HER3 antibody (e.g., 2C2-YTE antibody) or an antigen-binding fragment thereof in the treatment of head and neck cancer or colorectal cancer. In one embodiment, before administration of cetuximab, the patient is premedicated with an H1 antagonist (e.g., 50 mg of diphenhydramine) intravenously (IV) 30-60 minutes prior to the first dose. In one embodiment, cetuximab is administered as a 400 mg/m² initial dose over a 120-minute intravenous infusion. In one embodiment, the maximum infusion rate is 10 mg/min. In a specific embodiment, the initial dose of cetuximab is followed by weekly doses of 250 mg/m² infused over 60 minutes. In a specific embodiment, the 250 mg/m² weekly doses of cetuximab are continued for the duration of radiation therapy. In a specific embodiment, the 250 mg/m² weekly doses of cetuximab are continued until disease progression or until unacceptable toxicity. In one embodiment, the cetuximab dosage form is 100 mg/50 mL in a single use vial. In another embodiment, the cetuximab dosage form is 200 mg/100 mL in a single use vial. In certain aspects, doses of cetuximab can be administered per its package insert. In certain embodiments, prior to administration of the HER3 antibody, the cancer is resistant to treatment with a EGFR inhibitor (e.g., cetuximab or trastuzumab). In a particular embodiment, prior to administration of the HER3 antibody, the cancer is resistant to treatment with cetuximab. In another particular embodiment, prior to administration of the HER3 antibody, the cancer is resistant to treatment with trastuzumab. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, provided herein are methods for treating non-small cell lung cancer in a human subject comprising administering to the subject an anti-HER3 antibody (e.g., 2C2-YTE) or an antigen-binding fragment thereof at a dose of 15 mg/kg or 20 mg/kg every 21 days, in combination with an EGFR inhibitor such as erlotinib. In one embodiment, the dose of the anti-HER3 antibody or antigen-binding fragment thereof is 15 mg/kg. Provided herein are doses of erlotinib that are useful for combination therapy with an anti-HER3 antibody (e.g., 2C2-YTE antibody) or an antigen-binding fragment thereof in the treatment of NSCLC (non-small cell lung cancer). In one embodiment, erlotinib is administered at a dose of 150 mg/day. In one embodiment, erlotinib is administered at a dose of 100 mg/day. In a specific embodiment, erlotinib is administered at least one hour before food. In another specific embodiment, erlotinib is administered at least two hours after food. In a certain embodiment, the dose of erlotinib is reduced by 50 mg decrements. Non-limiting examples of circumstances where it may be appropriate to reduce the erlotinib dose by 50 mg decrements include: a patient who becomes dehydrated, has severe diarrhea, severe skin reactions, worsening living function, or other severe adverse reactions. In one embodiment, administration of erlotinib is continued until disease progression or unacceptable toxicity. In one embodiment, the dosage form of erlotinib is a 25 mg tablet. In another embodiment, the dosage form of erlotinib is a 100 mg tablet. In another embodiment, the dosage form of erlotinib is a 150 mg tablet. In certain aspects, doses of erlotinib can be administered per its package insert. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, provided herein are methods for treating melanoma, such as BRAF mutated melanoma, in a human subject comprising administering to the subject an anti-HER3 antibody (e.g., 2C2-YTE) or an antigen-binding fragment thereof at a dose of 15 mg/kg or 20 mg/kg every 21 days, in combination with a BRAF inhibitor such as vemurafenib. In one embodiment, the dose of the anti-HER3 antibody or antigen-binding fragment thereof is 15 mg/kg. Provided herein are doses of vemurafenib that are useful for combination therapy with an anti-HER3 antibody (e.g., 2C2-YTE antibody) or an antigen-binding fragment thereof in the treatment of melanoma with a BRAF V600E mutation. In one embodiment, vemurafenib is administered in a dose of 960 mg twice daily e.g., approximately every 12 hours. In a specific embodiment, the twice daily doses of vemurafenib are administered approximately twelve hours apart, with or without a meal. In a specific embodiment, vemurafenib is administered with a glass of water. In one embodiment, administration of vemurafenib is continued until disease progression or unacceptable toxicity occurs. In one embodiment, the dosage form of vemurafenib is a 240 mg film-coated tablet. In certain aspects, doses of vemurafenib can be administered per its package insert. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, provided herein are methods for treating melanoma, such as BRAF mutated melanoma, in a human subject comprising administering to the subject an anti-HER3 antibody (e.g., 2C2-YTE) or an antigen-binding fragment thereof at a dose of 15 mg/kg or 20 mg/kg every 21 days, in combination with a BRAF inhibitor such as dabrafenib. In one embodiment, the dose of the anti-HER3 antibody or antigen-binding fragment thereof is 15 mg/kg. In certain aspects, doses of dabrafenib can be administered per its package insert.

In particular aspects, provided herein are methods for treating melanoma, such as BRAF mutated melanoma, in a human subject comprising administering to the subject an anti-HER3 antibody (e.g., 2C2-YTE) or an antigen-binding fragment thereof at a dose of 15 mg/kg or 20 mg/kg every 21 days, in combination with a BRAF inhibitor such as vemurafenib or dabrafenib and a MEK inhibitor (e.g., trametinib). In one embodiment, the dose of the anti-HER3 antibody or antigen-binding fragment thereof is 15 mg/kg. In certain aspects, doses of BRAF inhibitors and MEK inhibitors can be administered per their respective package inserts. In one embodiment, the anti-HER3 antibody or antigen-binding fragment thereof is administered every 3 weeks, or 21 days. In certain embodiments, prior to administration of the HER3 antibody, the cancer is resistant to treatment with the BRAF inhibitor (e.g., vemurafenib or dabrafenib) and the MEK inhibitor (e.g., trametenib). In a particular embodiment, prior to administration of the HER3 antibody, the cancer is resistant to treatment with vemurafenib. In another particular embodiment, prior to administration of the HER3 antibody, the cancer is resistant to treatment with dabrafenib. In a particular embodiment, prior to administration of the HER3 antibody, the cancer is resistant to treatment with trametinib. In certain embodiments, the cancer comprises cells that overexpress a HER3 ligand, for example neuregulin, e.g., neuregulin 1.

In particular aspects, provided herein are methods for treating HER2 positive breast cancer or gastric cancer in a human subject comprising administering to the subject an anti-HER3 antibody (e.g., 2C2-YTE) or an antigen-binding fragment thereof at a dose of 15 mg/kg or 20 mg/kg every 21 days, in combination with a HER2 inhibitor such as trastuzumab. In one embodiment, the dose of the anti-HER3 antibody or antigen-binding fragment thereof is 15 mg/kg. Provided herein are doses of trastuzumab that are useful for combination therapy with an anti-HER3 antibody (e.g., 2C2-YTE antibody) or an antigen-binding fragment thereof in the treatment of HER2-overexpressing breast or gastric cancers. In one embodiment, trastuzumab is administered at an initial dose of 4 mg/kg over a 90 minute intravenous infusion. In a specific embodiment, the initial dose of trastuzumab is followed by 2 mg/kg of trastuzumab over a 30 minute intravenous infusion. In a specific embodiment, the 2 mg/kg dose of trastuzumab is repeated weekly for 52 weeks. In another embodiment, trastuzumab is administered at an initial dose of 8 mg/kg over a 90 minute intravenous infusion. In a specific embodiment, the initial dose of trastuzumab is followed by 6 mg/kg of trastuzumab over 30-90 minutes of intravenous infusion. In a specific embodiment, the 6 mg/kg dose of trastuzumab is infused every three weeks for 52 weeks. In one embodiment, trastuzumab is administered during or following paclitaxel, docetaxel, or docetaxel/carboplatin. In another embodiment, trastuzumab is administered within three weeks following completion of multi-modality, anthracycline-based chemotherapy regimens. In one embodiment, the dosage form of trastuzumab is a multidose vial nominally containing 440 mg of trastuzumab as a lyophilized, sterile powder. In certain aspects, doses of trastuzumab can be administered per its package insert. In certain embodiments, prior to administration of the HER3 antibody, the cancer is resistant to treatment with trastuzumab. In certain embodiments, the cancer comprises cells that overexpress a HER3 ligand, for example neuregulin, e.g., neuregulin 1. In a specific embodiment, the HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region. In a particular embodiment, the HER3 antibody or antigen-binding fragment thereof is a monoclonal, human anti-HER3 antibody or antigen-binding fragment thereof, comprising a heavy chain variable region (VH) comprising the amino acid sequence of SEQ ID NO:2; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising the amino acid sequence of SEQ ID NO:3; and a human lambda light chain constant region.

In particular aspects, provided herein are methods for treating cancer or gastric cancer in a human subject comprising administering to the subject an anti-HER3 antibody (e.g., 2C2-YTE) or an antigen-binding fragment thereof, for example, at a dose of 15 mg/kg or 20 mg/kg every 21 days or in an amount sufficient to achieve an antibody serum concentration of 50 μg/mL throughout a dosing interval (e.g., 1 week, 2 weeks, 3 weeks, or 4 weeks), in combination with anticancer agents, such as a chemotherapeutic agents (e.g., platinums (such as cisplatin or oxaliplatin), antimetabolites, alkylating agents, topoisomerase inhibitors, or microtubule targeting agents). Anticancer agents include drugs used to treat malignancies, such as cancerous growths. In one embodiment, the dose of the anti-HER3 antibody or antigen-binding fragment thereof is 15 mg/kg. Drug therapy can be used alone, or in combination with other treatments such as surgery or radiation therapy. Several classes of drugs can be used in cancer treatment, depending on the nature of the organ involved. For example, breast cancers are commonly stimulated by estrogens, and can be treated with drugs which inactive the sex hormones. Similarly, prostate cancer can be treated with drugs that inactivate androgens, the male sex hormone. Anti-cancer agents for use in certain methods described herein include, among others, antibodies (e.g., antibodies which bind IGF-1R, antibodies which bind EGFR, antibodies which bind HER2, antibodies which bind HER3, or antibodies which bind cMET), small molecules targeting IGF1R, small molecules targeting EGFR, small molecules targeting HER2, antimetabolites, alkylating agents, topoisomerase inhibitors, microtubule targeting agents, kinase inhibitors, protein synthesis inhibitors, immunotherapeutic agents, hormonal therapies, glucocorticoids, aromatase inhibitors, mTOR inhibitors, chemotherapeutic agents, Protein Kinase B inhibitors, Phosphatidylinositol 3-Kinase (PI3K) inhibitors, Cyclin Dependent Kinase (CDK) inhibitors, RLr9, CD289, enzyme inhibitors, anti-TRAIL, MEK inhibitors, etc.

In specific aspects the HER3-binding molecules described herein, e.g., antibodies or antigen-binding fragments thereof, can be administered in combination with a BRAF inhibitor (e.g., vemurafenib or dabrafenib) and a MEK (mitogen-activated protein kinase (MAPK) kinase, also known as MAPKK) inhibitor (e.g., selumetinib (AZD6244, ARRY-142866, AstraZeneca), WX-554 (Wilex), trametinib (MEKINIST®; GlaxoSmithKline), refametinib (Ardea Biosciences), E-6201 (Eisai), MEK-162 (Novartis), cobimetinib (GDC-0973; XL-518; Exelixis, Roche), TAK-733 (Takeda Phamaceuticals), binimetinib (Array BioPharma), PD-0325901 (Pfizer), pimasertib (MSC1936369; EMD Serono), MSC2015103 (EMD Serono), WX-554 (WILEX), or RO48987655 (CH4987655; CIF/RG7167; Chugai Pharmaceuticals)). In specific aspects, provided herein are methods for treating cancer (e.g., head and neck cancer, breast cancer, colon or colorectal cancer, thyroid cancer, lung cancer, pancreatic cancer, gastric cancer, ovarian cancer, or melanoma) comprising administering to a human subject in need thereof an anti-HER3 antibody or antigen binding fragment thereof described herein (e.g., antibody 2C2 or 2C2-YTE) and (i) a BRAF inhibitor (e.g., vemurafenib or dabrafenib), (ii) a MEK inhibitor (e.g., trametinib), or (iii) the combination of a BRAF inhibitor and a MEK inhibitor.

In some aspects, the HER3-binding molecules described herein can be administered in combination with MEK (mitogen-activated protein kinase (MAPK) kinase, also known as MAPKK) inhibitors, e.g., selumetinib (AZD6244, ARRY-142866, AstraZeneca), WX-554 (Wilex), trametinib (MEKINIST®; Glaxo SmithKline), refametinib (Ardea Biosciences), E-6201 (Eisai), and MEK-162 (Novartis). In a particular aspect, the combination of a MEK inhibitor and a HER3-binding molecule described herein is more efficacious than either agent alone. In a specific aspect, a HER3-binding molecule described herein is administered in combination with selumetinib.

Where the combined therapies comprise administration of an anti-HER3 binding molecule (e.g., 2C2-YTE) in combination with administration of another therapeutic agent, the methods described herein encompass coadministration, using separate formulations or a single pharmaceutical formulation, and consecutive administration in either order. In some aspects, the anti-HER3 antibodies described herein are administered in combination with other drugs, wherein the antibody or antigen-binding fragment, variant, or derivative thereof and the therapeutic agent(s) can be administered sequentially, in either order, or simultaneously (i.e., concurrently or within the same time frame).

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In one aspect, the cancer comprises the KRAS mutation. In specific aspects, the KRAS mutation is located at codon 12 of a human KRAS gene. In a particular aspect, the cancer comprising a KRAS mutation that is resistant to an RTK therapy, such as an EGFR mAb, e.g., cetuximab or panitumumab.

In one aspect, the cancer comprises a BRAF mutation. In specific aspects, the BRAF mutation is the BRAF V600E or V600K mutation. In a particular aspect, the cancer comprising a BRAF mutation that is resistant to an RTK therapy, such as an EGFR mAb, e.g., cetuximab or panitumumab.

In one aspect, provided herein are uses of anti-HER3 binding molecules, e.g., antibodies (e.g., CL16, 2C2 or 2C2-YTE antibodies) in combination with a BRAF inhibitor, in methods of managing or treating cancer (e.g., melanoma) in a subject in need thereof, in combination with a BRAF inhibitor (e.g., vemurafenib or dabrafenib).

### VII. Pharmaceutical Compositions and Administration Methods

Methods of preparing and administering anti-HER3 binding molecules, e.g., antibodies, or antigen-binding fragments, variants, or derivatives thereof described herein to a subject in need thereof are well known to or are readily determined by those skilled in the art. The route of administration of the anti-HER3 binding molecule, e.g., antibody, or antigen-binding fragment, variant, or derivative thereof can be, for example, oral, parenteral, by inhalation or topical. The term parenteral as used herein includes, e.g., intravenous, intraarterial, intraperitoneal, intramuscular, subcutaneous, rectal, or vaginal administration. However, in other methods compatible with the teachings herein, anti-HER3 binding molecules, e.g., antibodies, or antigen-binding fragments, variants, or derivatives thereof, described herein can be delivered directly to the site of the adverse cellular population thereby increasing the exposure of the diseased tissue to the therapeutic agent.

As discussed herein, anti-HER3 binding molecules, e.g., antibodies, or antigen-binding fragments, variants, or derivatives thereof described herein can be administered in a pharmaceutically effective amount for the in vivo treatment of HER3-expressing cell-mediated diseases such as certain types of cancers.

The pharmaceutical compositions used in the methods described herein can comprise pharmaceutically acceptable carriers, including, e.g., water, ion exchangers, proteins, buffer substances, and salts. Preservatives and other additives can also be present. The carrier can be a solvent or dispersion medium. Suitable formulations for use in the therapeutic methods disclosed herein are described in Remington's Pharmaceutical Sciences (Mack Publishing Co.) 16th ed. (1980). In some aspects, the HER3-binding molecules described herein are formulated in a refrigerator (2-8° C.) stable composition. In a particular aspect, the refrigerator stable composition comprises 25 mM histidine/histidine HCl, 205 mM sucrose, 0.02% polysorbate 80 at pH 6.0. In another particular aspect, the HER3-binding molecules described herein are formulated at 25-100 mg/ml in the refrigerator stable composition.

In any case, sterile injectable solutions can be prepared by incorporating an active compound (e.g., an anti-HER3 antibody, or antigen-binding fragment, variant, or derivative thereof, by itself or in combination with other active agents) in the required amount in an appropriate solvent followed by filtered sterilization. Further, the preparations can be packaged and sold in the form of a kit. Such articles of manufacture can have labels or package inserts indicating that the associated compositions are useful for treating a subject suffering from, or predisposed to a disease or disorder.

Parenteral formulations can be a single bolus dose, an infusion or a loading bolus dose followed with a maintenance dose. These compositions can be administered at specific fixed or variable intervals, e.g., once a day, or on an “as needed” basis.

The composition can be administered as a single dose, multiple doses or over an established period of time in an infusion. Dosage regimens also can be adjusted to provide the optimum desired response (e.g., a therapeutic or prophylactic response).

Therapeutically effective doses of the compositions of described herein, for treatment of HER3-expressing cell-mediated diseases such as certain types of cancers including e.g., colon cancer, lung cancer, thyroid cancer, gastric cancer, head and neck squamous cells cancer, melanoma, pancreatic cancer, prostate cancer, and breast cancer, vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated.

Factors influencing the mode of administration and the respective amount of at least one anti-HER3 binding molecule, e.g., antibody, antigen-binding fragment, variant or derivative thereof include, but are not limited to, the severity of the disease, the history of the disease, and the age, height, weight, health, and physical condition of the individual undergoing therapy. Similarly, the amount of anti-HER3 binding molecule, e.g., antibody, or fragment, variant, or derivative thereof, to be administered will be dependent upon the mode of administration and whether the subject will undergo a single dose or multiple doses of this agent.

Also provided herein is use of an anti-HER3 binding molecule, e.g., an antibody or antigen-binding fragment, variant, or derivative thereof, in the manufacture of a medicament for treating a type of cancer, including, e.g., colon cancer, lung cancer, gastric cancer, head and neck squamous cells cancer, thyroid cancer, melanoma, pancreatic cancer, prostate cancer, and breast cancer.

Also provided herein is use of an anti-HER3 binding molecule, e.g., antibody described herein, or antigen-binding fragment, variant, or derivative thereof, in the manufacture of a medicament for treating a subject for treating a type of cancer (e.g., melanoma, head and neck cancer, non-small cell lung cancer, colon cancer, breast cancer, or gastric cancer). In certain aspects, the medicament is used in a subject that has been pretreated with at least one other therapy. By “pretreated” or “pretreatment” is intended the subject has received one or more other therapies (e.g., been treated with at least one other anti-cancer therapy) prior to receiving the medicament comprising the anti-HER3 binding molecule, e.g., antibody or antigen-binding fragment, variant, or derivative thereof. It is not necessary that the subject was a responder to pretreatment with the prior therapy or therapies. Thus, the subject that receives the medicament comprising the anti-HER3 binding molecule, e.g., an antibody or antigen-binding fragment, variant, or derivative thereof could have responded, or could have failed to respond to pretreatment with the prior therapy, or to one or more of the prior therapies where pretreatment comprised multiple therapies.

Also provided herein is co-administration of an anti-HER3 binding molecule, e.g., antibody, or antigen-binding fragment, variant, or derivative thereof described herein and at least one other therapy (e.g., BRAF inhibitor, such as vemurafenib or dabrafenib; an EGFR inhibitor such as cetuximab or erlotinib; or a HER2 inhibitor such as trastuzumab). The anti-HER3 antibody and the at least one other therapy can be co-administered together in a single composition or can be co-administered together at the same time or overlapping times in separate compositions.

Also provided herein are use of an anti-HER3 binding molecule, e.g., antibody, or antigen-binding fragment, variant, or derivative thereof described herein, in the manufacture of a medicament for treating a subject for treating cancer, wherein the anti-HER3 binding molecule is administered before a subject has been treated with at least one other therapy.

### VIII. Diagnostics

Further provided herein is a diagnostic method useful during diagnosis of HER3-expressing cell-mediated diseases such as certain types of cancer including, e.g., colon cancer, lung cancer, gastric cancer, head and neck squamous cells cancer, melanoma, pancreatic cancer, prostate cancer, and breast cancer, which involves measuring the expression level of HER3 protein or transcript in tissue or other cells or body fluid from an individual and comparing the measured expression level with a standard HER3 expression level in normal tissue or body fluid, whereby an increase in the expression level compared to the standard is indicative of a disorder. Such samples can be out of the human or animal body.

The anti-HER3 antibodies described herein and antigen-binding fragments, variants, and derivatives thereof, can be used to assay HER3 protein levels in a biological sample using classical immunohistological methods known to those of skill in the art (e.g., see Jalkanen, et al., J. Cell. Biol. 101:976-985 (1985); Jalkanen et al., J. Cell Biol. 105:3087-3096 (1987)). Other antibody-based methods useful for detecting HER3 protein expression include immunoassays, such as the enzyme linked immunosorbent assay (ELISA), immunoprecipitation, or Western blotting. Suitable assays are described in more detail elsewhere herein.

By “assaying the expression level of HER3 polypeptide” is intended qualitatively or quantitatively measuring or estimating the level of HER3 polypeptide in a first biological sample either directly (e.g., by determining or estimating absolute protein level) or relatively (e.g., by comparing to the disease associated polypeptide level in a second biological sample). HER3 polypeptide expression level in the first biological sample can be measured or estimated and compared to a standard HER3 polypeptide level, the standard being taken from a second biological sample obtained from an individual not having the disorder or being determined by averaging levels from a population of individuals not having the disorder. As will be appreciated in the art, once the “standard” HER3 polypeptide level is known, it can be used repeatedly as a standard for comparison.

Further provided herein is a diagnostic method useful during diagnosis of HER3-expressing cell-mediated diseases such as certain types of cancer including, e.g., colon cancer, lung cancer, gastric cancer, head and neck squamous cells cancer, melanoma, pancreatic cancer, prostate cancer, and breast cancer, which involves measuring the activity level of HER3 protein in tissue or other cells or body fluid from an individual and comparing the measured activity level with a standard HER3 activity level in normal tissue or body fluid, whereby an increase in the activity level compared to the standard is indicative of a disorder.

Further provided herein is a diagnostic method useful during treatment of HER3-expressing cell-mediated diseases such as certain types of cancer including, e.g., colon cancer, lung cancer, gastric cancer, head and neck squamous cells cancer, melanoma, pancreatic cancer, prostate cancer, and breast cancer, which involves measuring the activity level of HER3 protein in tissue or other cells or body fluid from an individual during treatment of a HER3-expressing cell-mediated disease and comparing the measured activity level with a standard HER3 activity level in normal tissue or body fluid and/or comparing the measured activity level with a standard HER3 activity level in tissue or body fluid obtained from the individual prior to treatment, whereby a decrease in the activity level compared to the standard is indicative of an inhibition of HER3 activity.

By “assaying the activity level of HER3 protein” is intended qualitatively or quantitatively measuring or estimating the activity of HER3 protein in a first biological sample either directly (e.g., by determining or estimating absolute activity level) or relatively (e.g., by comparing to the activity level in a second biological sample). HER3 protein activity level in the first biological sample can be measured or estimated and compared to a standard HER3 protein activity, the standard being taken from a second biological sample obtained from an individual not having the disorder or being determined by averaging levels from a population of individuals not having the disorder or from an individual prior to treatment. As will be appreciated in the art, once the “standard” HER3 protein activity level is known, it can be used repeatedly as a standard for comparison. In certain aspects, the activity level of HER3 in a biological sample is measured or estimated or compared by detecting phosphorylated HER3 in a biological sample. In a specific aspect, the activity level of HER3 in a biological sample is measured or estimated or compared by detecting phosphorylated HER3 in a skin biopsy, wherein the skin is stimulated with HRG prior to or after biopsy.

Further provided herein, is a method of monitoring treatment of a human subject diagnosed with cancer, or response of a human subject diagnosed with cancer to treatment, with an anti-HER3 antibody (e.g., 2C2-YTE) or antigen-binding fragment thereof as a monotherapy or combination therapy, comprising measuring the expression level (e.g., concentration) of a pharmacodynamic marker in a biological sample from the human subject. In certain aspects, the pharmacodynamic marker is soluble HER3, TWEAK, TWEAK receptor, Neurexophilin-1, IL-18 binding protein, ANGL4, IL-18 receptor 1, or Kallikrein 7. In a certain aspect, the pharmacodynamic marker is measured prior to, and after, administration of one or more doses of an anti-HER3 antibody (e.g., 2C2-YTE) or antigen-binding fragment. In a certain aspect, the pharmacodynamic marker is measured prior to, and after, administration of the anti-HER3 antibody (e.g., 2C2-YTE) or antigen-binding fragment. In a particular aspect, the circulating concentration (e.g., circulating concentration in plasma) of a pharmacodynamic marker is measured by methods known in the art, for example, by ELISA or proteomic aptamer based approaches (e.g., aptamer-based multiplexed proteomic technology, see, e.g., Gold et al., 2010, PLOS ONE, 5:e15004). In a certain aspect, a pharmacodynamic marker is measured prior to, and after (e.g., 1 week, 2 weeks, 3 weeks, or 4 weeks), administration of an anti-HER3 antibody (e.g., 2C2-YTE) or antigen-binding fragment. In a certain aspect, a pharmacodynamic marker is measured prior to, and after (e.g., 1 week, 2 weeks, 3 weeks, or 4 weeks), administration of a first dose of an anti-HER3 antibody (e.g., 2C2-YTE) or antigen-binding fragment. In one aspect, the circulating concentration of a pharmacodynamic marker is measured, for example by ELISA. In specific aspects, changes (e.g., increase or decrease) in the concentration (e.g., circulating concentration) of a pharmacodynamic marker, such as soluble HER3, TWEAK, TWEAK receptor, Neurexophilin-1, IL-18 binding protein, ANGL4, IL-18 receptor 1, or Kallikrein 7, is indicative of treatment. In a particular aspect, an increase or decrease in soluble HER3 circulating concentration depending on the assay format following treatment with an anti-HER3 antibody (e.g., 2C2-YTE) or antigen-binding fragment thereof (e.g., administration of one or more doses) is indicative of treatment. In a particular aspect, an increase in soluble HER3 circulating concentration detected for example by ELISA, following treatment with the anti-HER3 antibody (e.g., 2C2-YTE) or antigen-binding fragment thereof is indicative of treatment. In a particular aspect, a decrease in soluble HER3 circulating concentration detected for example by proteomic aptamer based approaches (e.g., aptamer-based multiplexed proteomic technology, see, e.g., Gold et al., 2010, PLOS ONE, 5:e15004), following treatment with an anti-HER3 antibody (e.g., 2C2-YTE) or antigen-binding fragment thereof is indicative of treatment. In a certain aspect, an increase in circulating concentration of TWEAK or TWEAK receptor following treatment with the anti-HER3 antibody (e.g., 2C2-YTE) or antigen-binding fragment thereof is indicative of treatment. In a particular aspect, a decrease in circulating concentration of Neurexophilin-1, IL-18 binding protein, IL-18 receptor 1, ANGL4, or Kallinkrein 7 following treatment with the anti-HER3 antibody (e.g., 2C2-YTE) or antigen-binding fragment thereof is indicative of treatment.

By “biological sample” is intended any biological sample obtained from an individual, cell line, tissue culture, or other source of cells potentially expressing HER3. Methods for obtaining tissue biopsies and body fluids from mammals are well known in the art.

In some aspects, the bioactivity of a HER3 inhibitor (e.g., anti-HER3 antibody described herein and antigen-binding fragments, variants and derivatives thereof) administered to a subject can be detected using an ex-vivo assay. In particular aspects the ex-vivo assay comprises detecting the level of phosphorylated HER3 in a skin biopsy, wherein the skin is stimulated with HRG prior to or after biopsy. In a specific aspect matched skin biopsies are taken from a subject that has been administered the HER3 inhibitor. In a specific aspect, HRG is injected under a first area of the skin and a control buffer is injected under a second area of the skin of a subject administered the HER3 inhibitor, wherein after a desired amount of time (e.g., 10-60 minutes) a biopsy is taken from the first and second areas of the skin. In an alternative aspect, a first skin biopsy is treated with HRG and a second skin biopsy is treated with a control buffer, wherein the first and the second biopsies are matched skin biopsies taken from a subject that has been administered the HER3 inhibitor. In another specific aspect, the level of phosphorylated HER3 is detected in the skin biopsies. In certain aspects, the difference in the level of phosphorylated HER3 between the first (HRG treated) and the second (control buffer treated) biopsy is determined. In certain aspects, the skin biopsy is homogenized and the level of phosphorylated HER3 is detected by ELISA. In still other aspects, the levels of phosphorylated HER3 in the skin biopsies from a subject that has been administered the HER3 inhibitor is compared to the levels of phosphorylated HER3 in skin biopsies from a control subject that has not been administered the HER3 inhibitor, wherein a reduction in the level of phosphorylated HER3 in the skin biopsies of the subject that has been administered the HER3 inhibitor is a measure of the bioactivity of the HER3 inhibitor. In alternative aspects, the levels of phosphorylated HER3 in the skin biopsies from a subject that has been administered the HER3 inhibitor is compared to the levels of phosphorylated HER3 in skin biopsies from the same subject taken prior to the administration of the HER3 inhibitor, wherein a reduction in the level of phosphorylated HER3 in the skin biopsies of the subject after administration of the HER3 inhibitor is a measure of bioactivity of the HER3 inhibitor.

Further provided herein is a diagnostic method useful during diagnosis of diseases associated with BRAF mutations, such as BRAF V600E or V600K mutation, for example certain types of cancer including, e.g., colon or colorectal cancer, lung cancer, gastric cancer, head and neck squamous cells cancer, melanoma, thyroid cancer, pancreatic cancer, prostate cancer, and breast cancer, which involves detecting BRAF mutations in samples from a subject, such as nucleic acid or protein samples in tissue or other cells or body fluid from an individual. For example, detection of BRAF mutations in nucleic acid samples can be performed using routine methods known in the art, such as sequencing methods (e.g., Next Generation Sequencing, reverse transcription polymerase chain reaction (RT-PCR) or real-time PCR). Detection of BRAF mutations at the protein level can be performed with an antibody which specifically binds to an epitope of BRAF containing the BRAF mutation and does not bind to wildtype BRAF.

### IX. Immunoassays

Anti-HER3 binding molecules, e.g., antibodies or antigen-binding fragments thereof, variants, or derivatives thereof of the molecules described herein can be assayed for immunospecific binding by any method known in the art. The immunoassays that can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety).

HER3-binding molecules, e.g., anti-HER3 antibodies or antigen-binding fragments thereof, variants, or derivatives thereof of the molecules described herein, can be employed histologically, as in immunofluorescence, immunoelectron microscopy or non-immunological assays, for in situ detection of HER3 receptors or conserved variants or peptide fragments thereof. In situ detection can be accomplished by removing a histological specimen from a patient, and applying thereto a labeled HER3-binding molecule, e.g., an anti-HER3 antibody or antigen-binding fragment thereof, variant, or derivative thereof, preferably applied by overlaying the labeled HER3-binding molecule (e.g., and antibody or fragment) onto a biological sample. Through the use of such a procedure, it is possible to determine not only the presence of HER3, or conserved variants or peptide fragments, but also its distribution in the examined tissue. Those of ordinary skill will readily perceive that any of a wide variety of histological methods (such as staining procedures) can be modified in order to achieve such in situ detection.

The binding activity of a given lot of HER3-binding molecule, e.g., anti-HER3 antibody or antigen-binding fragment thereof, variant, or derivative thereof can be determined according to well-known methods. Those skilled in the art will be able to determine operative and optimal assay conditions for each determination by employing routine experimentation.

Methods and reagents suitable for determination of binding characteristics of an isolated HER3-binding molecule, e.g., anti-HER3 antibody or antigen-binding fragment thereof, variant, or an altered/mutant derivative thereof, are known in the art and/or are commercially available. Equipment and software designed for such kinetic analyses are commercially available (e.g., BIAcore, BIAevaluation software, GE Healthcare; KinExa Software, Sapidyne Instruments).

The practice of the methods described herein will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunnology (4th ed.; H. Freemand & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlan); Sambrook and Russell (2001) Molecular Cloning: A Laboratory Manual (Cold Spring Harbor Press); Lewin (2003) Genes VIII (Prentice Hal12003); Harlow and Lane (1988) Antibodies: A Laboratory Manual (Cold Spring Harbor Press); Dieffenbach and Dveksler (2003) PCR Primer (Cold Spring Harbor Press).

### EXAMPLES

### Example 1: Clinical Study for Monotherapy

A Phase I, open label, single-arm study of 2C2-YTE monotherapy was conducted to evaluate the safety, tolerability, antitumor activity, pharmacokinetics, and immunogenicity in adult subjects with advanced solid tumors refractory to standard therapy or for which no standard therapy exists. The study was conducted using the classic 3+3 dose escalation design, using 5 mg/kg, 10 mg/kg, and 20 mg/kg 2C2-YTE cohorts. 2C2-YTE was administered to all subjects as an IV infusion over 60 minutes every 21 days, subject to a maximum tolerated dose (MTD) being identified before all dose-escalation cohorts were completed. If two or more dose limiting toxicities (DLTs) would have been observed in cohort 1, enrollment would have been suspended and a new starting dose determined.

The MTD determination was based on the assessment during the dose-limiting toxicity (DLT) period, which is defined as the time from the first dose of 2C2-YTE to 21 days after the first dose of 2C2-YTE. Subjects were considered evaluable for assessment of DLT if they received a full dose of 2C2-YTE on Day 1 of Cycle 1 and completed the 3 week safety follow-up through the DLT evaluation period, or the subject experienced any DLT.

Initially, 3 or 4 subjects were enrolled for each dose cohort. If no DLTs were observed in the first 3 or 4 subjects during the DLT period, enrollment in the next higher dose cohort commenced.

Dose escalation to the next dose level was permitted only after the subjects enrolled in the current dose cohort completed the DLT observation period and all safety data was reviewed by a study-specific dose-escalation committee.

If 1 of 3 or 4 subjects in a dose cohort experienced a DLT, that dose cohort would have been expanded by 3 subjects to a total of 6-7 subjects. If no more than 1 of 6 subjects in the cohort experienced a DLT, dose escalation would have continued to the next higher dose cohort.

If 2 or more subjects in a dose cohort experienced a DLT during the DLT period, the MTD would have been exceeded and no further subjects would have been enrolled into that dose cohort. If 0 of 3 or 1 of 6 subjects experienced a DLT at the de-escalated dose, this would have been considered the MTD and an additional 6 to 9 subjects may have been enrolled, for a total of 12-16 subjects enrolled at the MTD.

Fourteen advanced cancer patients were enrolled: six with colorectal cancer, two with ovarian cancer, two with pancreatic cancer, and one each with bladder cancer, testicular cancer, endometrial cancer, and hepatocellular carcinoma. The median age of the patients was 61, and the age range was 34 to 88. The median number of prior regimens per patient was six, with the range from two to seventeen. The Eastern Cooperative Oncology Group (ECOG) status of the patients was 0 or 1, with 71% having an ECOG of 1. One hundred percent of patients were Caucasian, 9 patients were female and 5 were male. The average number of 21-day cycles of treatment per patient was one, with one patient remaining on treatment at cycle three.

During the trial, no DLTs were reported, and therefore, the MTD, which is based on the assessment of DLT, was not determined. The results of these studies show that 2C2-YTE is well tolerated in humans and did not achieve a MTD in cancer patients.

Although monitoring continues, the study-reported events were as follows: a total of 131 adverse events (AE) were reported, with 31 (24%) being related to 2C2-YTE, 7 (5%) reaching grade 2 or 3, and 0 reaching grade 4 or 5. Severity categories for adverse events are defined as follows: grade 1 for mild, grade 2 for moderate, grade 3 for severe, grade 4 for life threatening, and grade 5 for fatal. The most common adverse events related to 2C2-YTE were diarrhea (5, 16%), rash/mucositis/itch (3, 10%), and dry mouth/eye (3, 10%). The grade 2 adverse events related to 2C2-YTE were anemia (2, 6%), elevated aspartate aminotransferase (AST) (1, 3%), fatigue (1, 3%), and erythrodysesthesia (1, 3%). Of the three serious adverse events reported, only one was 2C2-YTE related (grade 3 diarrhea), with the other two being disease-related.

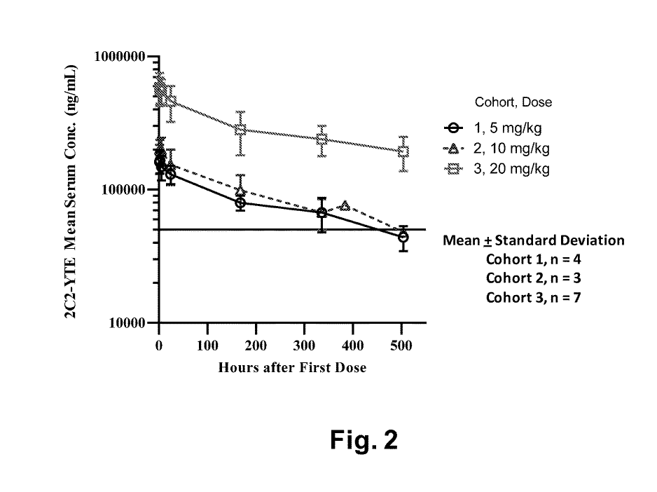

FIG. 2 shows the mean serum concentration (ng/mL) of 2C2-YTE at various time periods after treatment with the first dose in the 5 mg/kg, 10 mg/kg, and 20 mg/kg cohorts (cohorts 1, 2, and 3, respectively). Four patients were in cohort 1, three patients were in cohort 2, and seven patients were in cohort 3. The pharmacokinetics were found to be linear and the dose proportional. The doses for cohorts 2 and 3 were sufficient to achieve a mean antibody serum concentrations greater than a target serum concentration of 50 μg/mL. Cohort 3 achieved mean antibody serum concentrations greater than a target serum concentration of 50 μg/mL over a period of 3 weeks after antibody administration, and cohort 2 achieved mean antibody serum concentrations greater than a target serum concentration of 50 μg/mL over a period of approximately 20 days after antibody administration.

Pharmacodynamics Study

Evaluation of pharmacodynamic markers was performed by measuring plasma levels of such markers pre-dose and post-dose by ELISAs or proteomic aptamer based approaches (e.g., aptamer-based multiplexed proteomic technology, see, e.g., Gold et al., 2010, PLOS ONE, 5:e15004). Changes in soluble ErbB3 (sErbB3; see exemplary GenBank Accession No. NP_001005915.1) plasma levels (e.g., concentrations) were observed following treatment with 2C2-YTE for all three cohorts, e.g., a decrease in sErbB3 plasma levels were observed using a proteomic aptamer based approach. These evaluations indicate that accumulation of circulating soluble ErbB3 is not observed. Changes in a number of other pharmacodynamic markers monitored correlated with treatment with 2C2-YTE. For example, the plasma levels of TNF-related weak inducer of apoptosis (TWEAK; see exemplary GenBank Accession No. BAE16557.1) and TWEAK receptor (see exemplary GenBank Accession No. Q9NP84.1) increased in response to treatment with 2C2-YTE. The plasma levels of Neurexophilin-1 (see exemplary GenBank Accession No. AAH47505.1), IL-18 binding protein (see exemplary GenBank Accession No. BAA76374.1), angiopoietin-like 4 (ANGL4; see exemplary GenBank Accession No. AAH23647.1), IL-18 receptor 1 (see exemplary GenBank Accession No. AAH93977.1), and Kallikrein 7 (see exemplary GenBank Accession No. AAU04540.1) decreased in response to treatment with 2C2-YTE.

### Example 2: Clinical Study for Combination Therapy

An open-label, multiple-arm study is conducted to evaluate safety of 2C2-YTE in combination with other chemotherapeutic agents in specific cancers. Approximately 24 subjects are enrolled, and additional subjects may be enrolled to evaluate dose of 2C2-YTE (15 mg/kg or 20 mg/kg or a fixed dose of 1200 mg) or expand cohorts for further evaluation of a dose-limiting toxicity (DLT) as described. In one embodiment, the dose of the anti-HER3 antibody or antigen-binding fragment thereof is 15 mg/kg.

The clinical study involves combination therapy using 2C2-YTE with cetuximab (Arm A) erlotinib (Arm B), vemurafenib (Arm C) or trastuzumab (Arm D) in subjects with head and neck cancer or K-ras mutated negative, EGFR expressing colon cancer (Arm A), non small cell lung cancer (Arm B), BRAF mutated melanoma (Arm C) or HER2 positive breast or gastric cancer after having failed initial therapy for advanced or metastatic disease. Subjects with other cancers who may benefit from therapy with any of these combinations can be enrolled if no standard therapy exists.

Anti-HER3 antibody (2C2-YTE): A human immunoglobulin G1 lambda (IgG1λ) MAb that specifically binds ErbB3 and inhibits ErbB3 activity by multiple mechanisms, including ErbB3 degradation, internalization, and disruption of heterodimerization with known tumor growth drivers such as HER2 and possibly EGFR. The heavy chain CH2 domain of 2C2-YTE contains 3 amino acid substitutions that are referred to as YTE (M253Y/S255T/T257E; M252Y/S254T/T256E, according to the EU numbering system).

2C2-YTE is administered as an IV infusion over approximately 60 minutes (e.g., 60 minutes±5 minutes) on Day 1 at a dose of 20 mg/kg and repeated every 21 days. 2C2-YTE is diluted into 0.9% (w/v) saline to a final volume of 100 mL, in a 100 mL 0.9% saline IV infusion bag. Diluted 2C2-YTE is administered to the patient via IV infusion, through a low protein binding 0.2-μm in-line filter.

Cetuximab, erlotinib, vemurafenib and trastuzumab are given beginning on Day 2 at doses, for example as defined in the package insert. Weekly dosing of cetuximab and trastuzumab continue on Days 8 and 15 of Cycle 1. Beginning Cycle 2, 2C2-YTE and cetuximab or trastuzumab may be given on the same day, separated by 2 hours, provided they are both well tolerated. Premedication with an H1 antagonist or institutional premedication protocols may be administered prior to doses of cetuximab or trastuzumab. Each arm will enroll at least 6 subjects.

For Cycle 1, therapy with cetuximab, erlotinib, vemurafenib or trastuzumab should begin on Day 2. For cetuximab and trastuzumab on weekly schedules, dosing continues on Day 8 during Cycle 1. After Cycle 1, cetuximab or trastuzumab and 2C2-YTE may be given on the same day (starting with Cycle 2 on Day 1) provided they are separated by 2 hours. Oral medication including erlotinib and vemurafenib are given continuously.

Cetuximab is dosed according to package label using an initial loading dose of 400 mg/m² (Day 2 of Cycle 1) over 2 hours followed by weekly doses of 250 mg/m² over 60 minutes (starting at Day 8 and continuing weekly). Premedication with an H1 antagonist may be provided prior to cetuximab.

Erlotinib is dosed at 150 mg/day orally given at least 1 hour before or 2 hours after meals.

Vemurafenib is dosed at 960 mg approximately every 12 hours without regard to meals.

Trastuzumab is dosed according to package label using an initial dose of 4 mg/kg IV over 90 minutes (Day 2 of Cycle 1) followed by 2 mg/kg IV over approximately 30 minutes (starting on Day 8 of Cycle 1) every week for HER positive breast cancer, and 8 mg/kg IV over 90 minutes followed by 6 mg/kg over 30 minutes every 3 weeks for HER2-positive gastric cancer.

Dose modifications after Cycle 1 are allowed, for example per package insert guidelines for these combination therapies.

Subjects are enrolled in each arm independently. If 2 or more subjects in any arm experience a DLT, then 3 additional subjects will be treated with the combination at a dose of 15 mg/kg 2C2-YTE. DLTs in any arm will not result in a dose modification for other arms.

Subjects continue to receive treatment with 2C2-YTE until progressive disease, initiation of alternative anticancer treatment, unacceptable toxicity, or another reason to discontinue therapy.

Examples of endpoints include, but not limited to, the following are measured: assessments of adverse events (AEs), serious adverse events (SAEs), laboratory evaluations, radiological assessments, vital signs, electrocardiograms (ECGs), and physical examinations.

Examples of secondary endpoints include, but not limited to, the following are measured: pharmacokinetics, immunogenicity, and anti-tumor activity.

Below is a table summarizing a treatment regimen.

Preliminary results were obtained from the ongoing study described in Example 1, in which 2C2-YTE was administered at doses of 5, 10, 15, or 20 mg/kg once every three weeks as one hour IV infusions, and wherein 2C2-YTE was administered at a fixed dose of 1,200 mg once every three weeks as a one hour IV infusion. This has been followed by the ongoing study described in this Example, wherein six to twelve patients each receive 20 mg/kg of 2C2-YTE in combination with cetuximab, erlotinib, vemurafenib, or trastuzumab.

Evaluation of the soluble ErbB3 pharmacodynamic response of administration of 2C2-YTE alone or as part of these combination treatments demonstrates that the combinations do not influence the accumulation of circulating soluble ErbB3.

Pharmacokinetics of 2C2-YTE alone and in the combination treatments was evaluated by generating mean serum concentration time profiles of 2C2-YTE when administered alone and when administered in combination with cetuximab, erlotinib, vemurafenib or trastuzumab. The results demonstrate that the combinations do not influence 2C2-YTE serum concentration and show that all patients achieved a serum concentration of at least 50 μg/mL over a period of approximately 20 days after antibody administration.

While the study is ongoing and monitoring continues, stable disease has been observed in a patient with BRAF-mutated colorectal cancer being treated with 2C2-YTE and vemurafenib and in a patient with BRAF-mutated non-small cell lung cancer being treated with 2C2-YTE and vemurafenib. Further, while the result must be confirmed, an unquantified preliminary response has been observed in a patient with BRAF-mutated non-small cell lung cancer being treated with 2C2-YTE and vemurafenib. Moreover, while the result must yet be confirmed, a preliminary complete response has been observed in a patient with metastatic squamous cell carcinoma of the head and neck receiving 2C2-YTE in combination with cetuximab. The patient had a neck mass that recurred following surgery. Post-operatively, the patient was treated with cetuximab until the mass was discovered. Despite demonstrating this clinical resistance to cetuximab, the patient's tumor, which was 1.3 cm in largest diameter on a baseline CT scan and palpable prior to the beginning of treatment with 2C2-YTE and cetuximab, was no longer identified on a follow-up CT scan obtained after 3 cycles of treatment with 2C2-YTE and cetuximab, and could not be felt by the physician or patient. The patient has thus far received six cycles of 2C2-YTE. A follow-up CT scan will confirm the complete response if the lesion can no longer be seen.

### Example 3: Clinical Study for BRAF Mutant Radioiodine-Refractory Thyroid Cancer

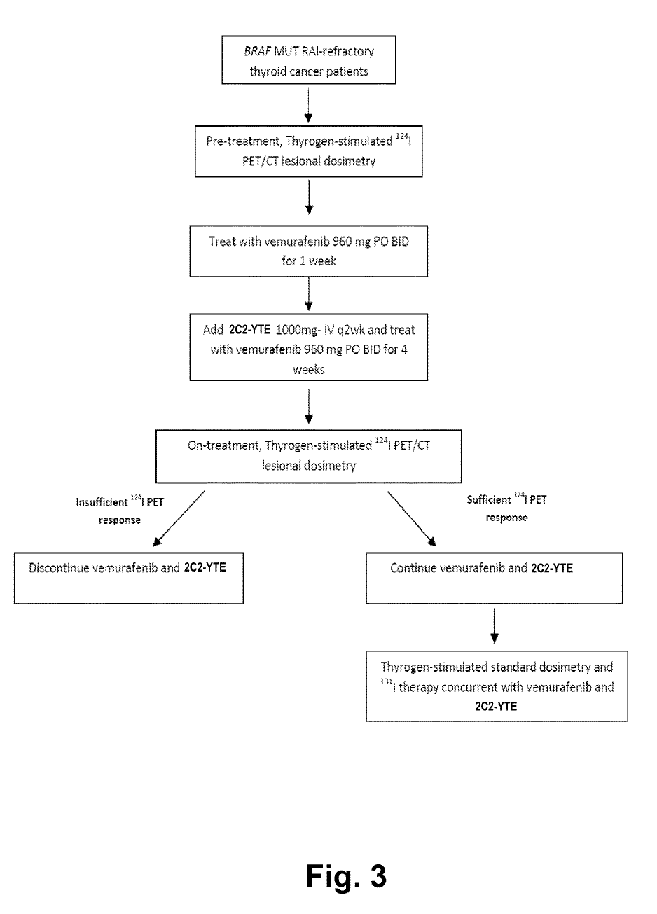

A study is conducted to assess restoration of iodine incorporation in BRAF mutant (MUT), radioiodine-refractory (RAIR) thyroid cancer patients receiving 2C2-YTE and vemurafenib. An administration schema for this study, in the form of a flow-chart, is shown in FIG. 3. Approximately 10 patients are enrolled with confirmed BRAF MUT, RAIR thyroid cancer. Eligible patients with BRAF MUT, RAIR thyroid cancer undergo human recombinant thyroid stimulating hormone (TSH; rhTSH or Thyrogen (Genzyme))-stimulated ¹²⁴I PET/CT lesional dosimetry to quantify the baseline RAI avidity of index metastatic lesion(s). Patients then receive vemurafenib 960 mg oral BID for a lead-in phase of 1 week followed by the addition of 2C2-YTE at a dose of 1000 mg every 2 weeks for approximately 4 weeks, after which a second Thyrogen-stimulated ¹²⁴I PET/CT lesional dosimetry is performed.

For patients whose tumor(s) demonstrate sufficient iodine incorporation warranting ¹³¹I therapy (i.e., if the patient demonstrates that ≧2,000 cGy can be delivered to at least one tumor with <300 mCi of ¹³¹I, based on the results of ¹²⁴I PET/CT lesional dosimetry), Thyrogen-stimulated standard dosimetry is performed and therapeutic ¹³¹I is administered concurrently with vemurafenib and 2C2-YTE. Specifically, patients who demonstrate sufficient iodine incorporation receive an additional dose of 2C2-YTE 1000 mg and will have sufficient dose levels of 2C2-YTE throughout the performance of standard dosimetry and administration of ¹³¹I. Vemurafenib is continued for two days following the administration of ¹³¹I. Subsequent to discontinuation of vemurafenib, tumor assessments are conducted with serial radiologic scan(s) and serum thyroglobulins. For patients whose tumors fail to demonstrate sufficient iodine incorporation to warrant ¹³¹I therapy after vemurafenib and 2C2-YTE following ¹²⁴I PET/CT dosimetry, an additional dose of 2C2-YTE is not administered and vemurafenib is discontinued.

The primary objectives of the study are (i) to determine the proportion of patients with BRAF MUT, RAIR thyroid cancer in which the combination of vemurafenib and 2C2-YTE increases tumoral iodine incorporation sufficient to warrant ¹³¹I treatment as determined by ^{124I} PET/CT lesional dosimetry, and (ii) to evaluate the safety and tolerability of the combination of vemurafenib and 2C2-YTE. The secondary objectives are (i) to evaluate the effect of vemurafenib and 2C2-YTE on enhancing ¹³¹I activity by determining the objective response rate (ORR) by Response Evaluation Criteria in Solid Tumors (RECIST) v1.1 criteria at 6 months following treatment with vemurafenib and 2C2-YTE plus ¹³¹I, (ii) to evaluate the effect of vemurafenib and 2C2-YTE on enhancing ¹³¹I activity by determining the proportion of patients alive at 6 months without disease progression by RECIST v1.1 criteria following treatment with vemurafenib and 2C2-YTE plus ¹³¹I, (iii) to evaluate changes in serum thyroglobulin levels in patients treated with ¹³¹I in co-administration with vemurafenib and 2C2-YTE, and (iv) to evaluate the safety and tolerability of vemurafenib and 2C2-YTE in combination with ¹³¹I.

Patients who participate in this study should have a histologically or cytologically confirmed thyroid carcinoma of follicular origin (including papillary, follicular, or poorly differentiated subtypes and their respective variants) and at least one thyroid tumor (primary tumor, recurrent tumor, or metastasis) that possesses a BRAF mutation at V600. Patients should have RAIR disease on structural imaging, defined as any one of the following: (a) a metastatic lesion that is not radioiodine-avid on a diagnostic radioiodine scan performed up to 2 years prior to enrollment in the current study, or (b) a radioiodine-avid metastatic lesion which remained stable in size or progressed despite radioiodine treatment 6 months or more prior to entry in the study, or (c) the presence of at least one fluorodeoxyglucose (FDG) avid lesion with a maximum standardized update value (SUVmax)>5.

Treatment Regimen:

All patients start vemurafenib 960 mg orally twice daily on Week 1, Day 6 after completion of the first ¹²⁴I PET/CT lesional dosimetry process (which begins on Week 1, Day 1). An infusion of 2C2-YTE of 1000 mg is given during week 3 and week 5. 2C2-YTE is diluted into 0.9% (w/v) saline to a final volume of 100 mL, in a 100 mL 0.9% saline IV infusion bag. Diluted 2C2-YTE is administered to the patient via IV infusion, through a low protein binding 0.2-μm in-line filter. IV infusion is performed over 60 minutes. If vemurafenib treatment requires interruption, patients will attempt to remain on vemurafenib continuously for a minimum of 7 days prior to the second ¹²⁴I PET/CT scan for lesional dosimetry assessment. If the second ¹²⁴I PET/CT demonstrates that ≧2,000 cGy can be delivered to at least one tumor with <300 mCi of ¹³¹I, the patient remains on vemurafenib, receives an additional dose of 2C2-YTE on week 7, and therapeutic ¹³¹I is administered during week 8. Vemurafenib is discontinued two days following ¹³¹I administration.

Safety Evaluations:

Safety evaluations include assessments of adverse events (AEs), serious adverse events (SAEs), laboratory evaluations, radiological assessments, vital signs, electrocardiograms (ECGs), and physical examinations. The occurrence of AEs, abnormal laboratory values, and SAEs reported are summarized for all patients who received vemurafenib and 2C2-YTE. Adverse events and SAEs are graded according to the National Cancer Institute Common Terminology Criteria for Adverse Events (NCI CTCAE) v4.03 and described by system organ class using the Medical Dictionary for Regulatory Activities (MedDRA) preferred term, severity, and relationship (causality) to 2C2-YTE. The immunogenic potential of 2C2-YTE is assessed by summarizing the number and percentage of patients who develop detectable antidrug antibody (ADA). For any confirmed ADA-positive patients, the impact of ADA on PK is assessed if data allow. Safety is assessed continuously and treatment may continue only if treatment is considered safe and tolerable. Specific safety stopping rules include number and severity of AEs and SAEs, physical findings, and any impact on therapy schedule due to treatments.

### Example 4: 2C2-YTE Resensitizes B-Raf Mutant Tumor Cells To Treatment with BRAF and MEK Inhibitors

The results presented herein demonstrate that treatment of B-Raf mutated (V600E or V600K) tumor cells with BRAF or MEK inhibitors (vemurafenib or trametinib), results in increased total and cell-surface levels of ErbB3. The increased number of NRG-binding sites renders the cells hypersensitive to NRG/ErbB3 signaling, as demonstrated by a several-fold increase in the maximum levels of phosphorylated ErbB3 and AKT. Such an increase in maximal phosphorylation values, however, does not alter the potency of 2C2-YTE. Importantly, the proliferation data described herein demonstrate that while NRG signaling via ErbB3 can robustly attenuate the effects of BRAF and MEK inhibitors, treatment with 2C2-YTE can restore the tumor cells' sensitivity to these inhibitors, thereby resensitizing the cells to BRAF or MEK inhibitors.

First, the effect of BRAF and MEK inhibitors on ErbB3 protein levels was measured in four B-Raf mutated (V600E or V600K) cell lines. Colorectal cancer line (HT29) and melanoma lines (A275, A2058 and WM115) were treated for 24 hours with 10 or 100 nM of trametinib and 100 to 1000 nM vemurafenib (which partially and completely inhibit their respective targets). Cells were treated ±10 nM neuregulin (NRG) for 10 minutes at 37° C. The samples were used to assess the expression levels of ErbB3. Western immunoblot analysis demonstrated that treatment with the BRAF and MEK inhibitors results in the upregulation of ErbB3 protein levels.

The effects of trametinib and vemurafenib on cell-surface expressed ErbB3 were also monitored. HT29, A375, A2058 and WM115 cells were treated with 100 nM trametinib or 1 μM vemurafenib for 24 hours at 37° C. Fluorescently-labeled 2C2-YTE was used as a probe specific for ErbB3, and fluorescently-labeled NRG was used as an alternative probe for ErbB3 binding sites. Specific binding was determined by calculating the mean fluorescent intensity ratio of total binding (labeled probe alone) divided by the non-specific binding (labeled probe with excess of unlabeled probe). Values should be reported as the binding ratio of drug-treated samples divided by the untreated sample. This analysis demonstrated that treatment with the BRAF and MEK inhibitors resulted in upregulation of ErbB3 cell surface levels.

To determine whether the observed increase in ErbB3 cell-surface levels upon treatment with BRAF and MEK inhibitors also correlates with increased NRG sensitivity, trametinib-treated (100 nM) and vemurafenib-treated (1 μM) cells were stimulated with a titration of NRG, and its effects on ErbB3 and downstream AKT phosphorylation levels relative to an untreated control were assessed. ErbB3 and AKT phosphorylation ELISA assays demonstrated that cells treated with the BRAF and MEK inhibitors become hypersensitive to the effects of NRG; addition of NRG results in a several-fold increase of phosphorylation of ErbB3 and AKT relative to untreated cells.

To determine whether trametinib or vemurafenib-induced sensitivity to NRG affects the IC₅₀ values of 2C2-YTE, HT29, A375, A2058 and WM115 cells were treated with each inhibitor (trametinib (100 nM) and vemurafenib (1 μM)) as described above followed by an incubation with increasing concentrations of 2C2-YTE. Cells were then stimulated with 10 nM of NRG. The cell-based ErbB3 phosphorylation IC50 of 2C2-YTE was then determined, which demonstrated that despite the trametinib- or vemurafenib-induced sensitivity to NRG, the potency of 2C2-YTE is insensitive to pre-treatment of cells with these BRAF or MEK inhibitors.

To investigate whether NRG signaling attenuates the anti-proliferative effects of trametinib or vemurafenib, cells were exposed to the drugs in the presence or absence of NRG. In addition, 2C2-YTE was introduced in a separate set of samples. Cell proliferative assays were performed in all four B-Raf mutated cell lines (H29, A375, A2058 and WM115). The results of these experiments demonstrate that while NRG attenuates the anti-proliferative effects of the BRAF and MEK inhibitors, making them resistant to such treatment, addition of 2C2-YTE actually resensitizes the tumor cells to the anti-proliferative effects of vemurafenib and trametinib.

### INCORPORATION BY REFERENCE

All of the references cited above, as well as all references cited herein, are incorporated herein by reference in their entireties.

All publications, including patent application publications, and patents mentioned herein are hereby incorporated by reference in their entirety as if each individual publication or patent was specifically and individually indicated to be incorporated by reference.

What is claimed is:

1. A method of treating cancer in a human subject in need thereof, comprising (i) administering to the subject an amount of an anti-HER3 antibody or antigen-binding fragment thereof, which specifically binds to an epitope within the extracellular domain of HER3, wherein the amount of the anti-HER3 antibody or antigen-binding fragment thereof is sufficient to achieve a Cₘᵢₙ of 50 μg/mL or greater antibody serum concentration or an antibody serum concentration of 50 μg/mL or greater throughout a dosing interval; wherein the anti-HER3 antibody or antigen-binding fragment comprises a VL comprising SEQ ID NO: 3 and a VH comprising SEQ ID NO: 2, and a human IgG constant region, wherein the human IgG constant region, comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E); and
(ii) administering to the subject a therapeutically effective amount of a BRAF inhibitor, an EGFR inhibitor, a MEK inhibitor, or a HER2 inhibitor.

2. A method of treating cancer in a human subject, comprising (i) administering to the subject a therapeutically effective amount of a BRAF inhibitor, an EGFR inhibitor, or a HER2 inhibitor; and (ii) administering to the human subject, in a dose of 15 mg/kg or 20 mg/kg, an anti-HER3 antibody or antigen-binding fragment thereof which specifically binds to an epitope within the extracellular domain of HER3, wherein the anti-HER3 antibody specifically binds to the same HER3 epitope as an antibody or antigen-binding fragment thereof comprising the heavy chain variable region (VH) and light chain variable region (VL) of CL16 or 2C2.

3. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3, and wherein the anti-HER3 antibody competitively inhibits HER3 binding by an antibody or antigen-binding fragment thereof comprising the VH and VL of CL16 or 2C2.

4. The method of claim 2 or claim 3, wherein the VH and VL of CL16 comprise SEQ ID NOs: 2 and 1, respectively, and the VH and VL of 2C2 comprise SEQ ID NOs: 2 and 3, respectively.

5. The method of any one of claims 1 to 4, wherein the anti-HER3 antibody or antigen-binding fragment thereof is affinity matured.

6. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VL, wherein the VL comprises the amino acid sequence:
[FW₁]X₁GSX₂SNIGLNYVS[FW₂]RNNQRPS[FW₃]AAWDDX₃X₄X₅GEX₆
[FW₄]
wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A).

7. The method of claim 6, wherein FW₁ comprises SEQ ID NO: 40 or 44, FW₂ comprises SEQ ID NO: 41, FW₃ comprises SEQ ID NO: 42, and FW₄ comprises SEQ ID NO: 43.

8. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VH, wherein the VH comprises the amino acid sequence:
[FW₅]YYYMQ[FW₆]X₇IGSSGGVTNYADSVKG[FW₇]VGLGDAFDI[FW₈]
wherein [FW₅], [FW₆], [FW₇] and [FW₈] represent VH framework regions, and wherein
X₇ represents amino acid residues Tyrosine (Y), Isoleucine (I) or Valine (V).

9. The method of claim 8, wherein FW₅ comprises SEQ ID NO: 36, FW₆ comprises SEQ ID NO: 37, FW₇ comprises SEQ ID NO: 38 and FW₈ comprises SEQ ID NO: 39.

10. A method of treating cancer in a human subject, comprising (i) administering to the subject a therapeutically effective amount of a BRAF inhibitor, an EGFR inhibitor, or a HER2 inhibitor; and (ii) administering to the subject at a dose of 15 mg/kg or 20 mg/kg an anti-HER3 antibody or antigen-binding fragment thereof which specifically binds to HER3, wherein the anti-HER3 antibody or antigen-binding fragment comprises an antibody VL and an antibody VH, wherein the VL comprises the amino acid sequence:
[FW₁]X₁GSX₂SNIGLNYVS[FW₂]RNNQRPS[FW₃]AAWDDX₃X₄X₅GEX₆
[FW₄]
wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and
wherein the VH comprises the amino acid sequence:
[FW₅]YYYMQ[FW₆]X₇IGSSGGVTNYADSVKG[FW₇]VGLGDAFDI[FW₈]
wherein [FW₅], [FW₆], [FW₇] and [FW₈] represent VH framework regions, and wherein
X₇ represents amino acid residues Tyrosine (Y), Isoleucine (I) or Valine (V).

11. The method of claim 10, wherein FW₁ comprises SEQ ID NO: 40 or 44, FW₂ comprises SEQ ID NO: 41, FW₃ comprises SEQ ID NO: 42, FW₄ comprises SEQ ID NO: 43, FW₅ comprises SEQ ID NO: 36, FW₆ comprises SEQ ID NO: 37, FW₇ comprises SEQ ID NO: 38, and FW₈ comprises SEQ ID NO: 39.

12. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VL, wherein the VL comprises a VL complementarity determining region-1 (VL-CDR1) amino acid sequence identical to, or identical except for four, three, two or one amino acid substitutions to: SEQ ID NO: 18, SEQ ID NO: 19, or SEQ ID NO: 20.

13. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VL, wherein the VL comprises a VL complementarity determining region-2 (VL-CDR2) amino acid sequence identical to, or identical except for four, three, two or one amino acid substitutions to SEQ ID NO: 21.

14. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen binding fragment thereof which specifically binds to HER3 comprising an antibody VL, wherein the VL comprises a complementarity determining region-3 (VL-CDR3) amino acid sequence identical to, or identical except for four, three, two, or one amino acid substitutions to: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

15. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VH, wherein the VH comprises a complementarity determining region-1 (VH-CDR1) amino acid sequence identical to, or identical except for four, three, two, or one amino acid substitutions to SEQ ID NO: 31.

16. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VH, wherein the VH comprises a complementarity determining region-2 (VH-CDR2) amino acid sequence identical to, or identical except for four, three, two, or one amino acid substitutions to: SEQ ID NO: 32, SEQ ID NO: 33, or SEQ ID NO: 34.

17. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VH, wherein the VH comprises a complementarity determining region-3 (VH-CDR3) amino acid sequence identical to, or identical except for four, three, two, or one amino acid substitutions to SEQ ID NO: 35.

18. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 18, 21 and 22, SEQ ID NOs: 18, 21, and 26, SEQ ID NOs: 18, 21, and 27, SEQ ID NOs: 20, 21, and 22, SEQ ID NOs: 19, 21, and 22, SEQ ID NOs: 18, 21, and 25, SEQ ID NOs: 18, 21, and 28, SEQ ID NOs: 18, 21, and 29, SEQ ID NOs: 18, 21, and 30, SEQ ID NOs: 18, 21, and 23, SEQ ID NOs: 19, 21, and 23, SEQ ID NOs: 20, 21, and 23, SEQ ID NOs: 18, 21, and 24, or SEQ ID NOs: 18, 21, and 25, respectively.

19. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3 comprising an antibody VH, wherein the VH comprises VH-CDR1, VH-CDR2, and VH-CDR3 amino acid sequences identical to, or identical except for four, three, two, or one amino acid substitutions in one or more of the VH-CDRS to: SEQ ID NOs: 31, 32 and 35, SEQ ID NOs: 31, 33, and 35, or SEQ ID NOs: 31, 34, and 35, respectively.

20. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an antibody or antigen binding fragment thereof which specifically binds to HER3 comprising a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

21. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen binding fragment thereof which specifically binds to HER3, wherein the anti-HER3 antibody or antigen-binding fragment comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

22. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3, wherein the anti-HER3 antibody or antigen-binding fragment comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

23. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3, wherein the anti-HER3 antibody or antigen-binding fragment comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

24. A method of treating cancer in a human subject in need thereof, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, of an anti-HER3 antibody or antigen-binding fragment thereof which specifically binds to HER3, wherein the anti-HER3 antibody or antigen-binding fragment thereof comprises a VL comprising the VL consensus sequence provided in Table 3 and a VH comprising the VH consensus sequence provided in Table 3.

25. A method of treating cancer in a human subject in need thereof, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to HER3, wherein the anti-HER3 antibody or antigen-binding fragment comprises a VL comprising SEQ ID NO: 3 and a VH comprising SEQ ID NO: 2.

26. The method of any one of claims 2-25, wherein the anti-HER3 antibody or antigen-binding fragment comprises a human heavy chain constant region or fragment thereof.

27. The method of claim 26, wherein the heavy chain constant region or fragment thereof is an IgG constant region.

28. The method of claim 27, wherein the IgG constant region is selected from an IgG1 constant region, an IgG2 constant region, an IgG3 constant region and an IgG4 constant region.

29. The method of claim 27, wherein the IgG constant region is an IgG1 constant region.

30. The method of any one of claims 2-29, wherein the anti-HER3 antibody or antigen-binding fragment comprises a light chain constant region selected from the group consisting of a human kappa constant region and a human lambda constant region.

31. The method of claim 30, wherein the anti-HER3 antibody or antigen-binding fragment comprises a human lambda constant region.

32. The method of any one of claims 26-31, wherein the IgG constant domain comprises one or more amino acid substitutions relative to a wild-type IgG constant domain wherein the modified IgG has an increased half-life compared to the half-life of an IgG having the wild-type IgG constant domain.

33. The method of claim 32, wherein the IgG constant domain comprises one or more amino acid substitutions of amino acid residues at positions 251-257, 285-290, 308-314, 385-389, and 428-436, wherein the numbering is according to the EU index as set forth in Kabat.

34. The method of claim 33, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions,
wherein the numbering is according to the EU index as set forth in Kabat.

35. The method of claim 33, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

36. The method of any one of claim 33, 34, or 35, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

37. The method of claim 36, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

38. The method of claim 37, wherein the amino acid at position 257 is substituted with Leucine (L), and the amino acid at Kabat position 434 is substituted with Tyrosine (Y), and wherein the numbering is according to the EU index as set forth in Kabat.

39. The method of claim 38, wherein the amino acid at Kabat position 428 is substituted with Leucine (L), and the amino acid at Kabat position 434 is substituted with Serine (S).

40. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to an epitope within the extracellular domain of HER3, wherein the anti-HER3 antibody or antigen binding fragment comprises an antibody VL of SEQ ID NO:3, an antibody VH of SEQ ID NO: 2, and an IgG1 constant region of SEQ ID 46.

41. A method of treating cancer in a human subject, comprising (i) administering to the subject a therapeutically effective amount of a BRAF inhibitor, an EGFR inhibitor, or a HER2 inhibitor; and (ii) administering to the subject, in a dose of 15 mg/kg or 20 mg/kg, an anti-HER3 antibody or antigen-binding fragment thereof which specifically binds to an epitope within the extracellular domain of HER3, consisting of an antibody VL of SEQ ID NO: 3, an antibody VH of SEQ ID NO: 2, and an IgG1 constant region of SEQ ID 46.

42. The method of claim 26 or claim 27 wherein the human IgG constant region, comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

43. The method of any one of claims 1-42, wherein the antibody is a fully human antibody, a humanized antibody, a chimeric antibody, a monoclonal antibody, a polyclonal antibody, a recombinant antibody, a multispecific antibody, or an antigen-binding fragment thereof.

44. The method of any one of claims 1-43, which antigen-binding fragment is Fv, Fab, F(ab′)2, Fab′, dsFv, scFv, and sc(Fv)2.

45. The method of any one of claims 1-44, which anti-HER3 antibody or antigen-binding fragment is conjugated to at least one heterologous agent.

46. The method of any one of claims 1-45, wherein the anti-HER3 antibody or antigen-binding fragment can bind to human HER3, cynomolgus monkey HER3, and mouse HER3.

47. The method of any one of claims 1-46, further comprising administering to the subject a therapeutic effective amount of (i) a MEK inhibitor, or (ii) a MEK inhibitor and a BRAF inhibitor.

48. The method of claim 47, wherein the MEK inhibitor is trametinib.

49. The method of any one of claims 1-48, wherein the cancer is selected from the group consisting of melanoma, thyroid cancer, colon cancer, non small cell lung cancer, gastric cancer, breast cancer, and head and neck cancer.

50. The method of claim 49, wherein the cancer is squamous cell carcinoma of the head and neck (SCCHN).

51. The method of claim 50, wherein the cancer is human papillomavirus (HPV) positive SCCHN.

52. The method of claim 50, wherein the cancer is HPV negative SCCHN.

53. The method of claim 49, wherein the cancer comprises cells comprising a KRAS mutation.

54. The method of claim 53, wherein the KRAS mutation comprises a mutation of codon 12 of a human KRAS gene.

55. The method of any one of claims 1-54, wherein the human subject is administered a BRAF inhibitor.

56. The method of any one of claims 1-55, wherein the BRAF inhibitor is vemurafenib.

57. The method of any one of claims 1-56, wherein the cancer comprises cells comprising a BRAF mutation.

58. The method of any one of claim 57, wherein the BRAF mutation is the BRAF V600E mutation.

59. The method of any one of claims 1-58, wherein the BRAF inhibitor is an antibody or antigen binding fragment thereof.

60. The method of claim any one of claims 1-59, wherein the human subject has been diagnosed with BRAF mutated melanoma.

61. The method of claim 60, wherein the human subject is administered (i) the anti-HER3 antibody or antigen-binding fragment by intravenous (IV) infusion on day 1 and repeated every 21 days; and (ii) vemurafenib at a dose of 960 mg approximately every 12 hours starting on day 2.

62. The method of any one of claims 1-46, wherein the human subject is administered an EGFR inhibitor.

63. The method of claim 62, wherein the EGFR inhibitor which is cetuximab.

64. The method of claim 63, wherein the human subject has been diagnosed with SCCHN.

65. The method of claim 64, wherein the SCCHN is HPV positive.

66. The method of claim 64, wherein the SCCHN is HPV negative.

67. The method of any one of claims 64-66, wherein the SCCHN is EGFR-expressing SCCHN.

68. The method of claim 63, wherein the human subject has been diagnosed with colon cancer.

69. The method of any one of claims 63-68, wherein the human subject is administered (i) the anti-HER3 antibody or antigen-binding fragment by intravenous (IV) infusion on day 1 and repeated every 21 days; and (ii) cetuximab at an initial loading dose of 400 mg/m² on day 2 over approximately 2 hours followed by weekly doses of 250 mg/m² over approximately 60 minutes starting on day 8 and continuing weekly.

70. The method of any one of claims 63-68, wherein the human subject is administered (i) the anti-HER3 antibody or antigen-binding fragment by intravenous (IV) infusion on day 1 and repeated every 21 days; and (ii) cetuximab on day 1 at a dose of 400 mg/m² or 250 mg/m² weekly or at an initial loading dose of 400 mg/m² followed by weekly doses of 250 mg/m².

71. The method of claim 70, wherein the anti-HER3 antibody or antigen-binding fragment and the cetuximab are administered approximately 2 hours apart on the same day.

72. The method of any one of claims 1-46, wherein the human subject is administered an EGFR inhibitor which is erlotinib.

73. The method of claim 72, wherein the human subject has been diagnosed with non-small cell lung cancer.

74. The method of claim 72 or 73, wherein the human subject is administered (i) the anti-HER3 antibody or antigen-binding fragment by intravenous (IV) infusion on day 1 and repeated every 21 days; and (ii) erlotinib on day 2 at a dose of 150 mg/day orally.

75. The method of claim 72 or 73, wherein the human subject is administered (i) the anti-HER3 antibody or antigen-binding fragment by intravenous (IV) infusion on day 1 and repeated every 21 days; and (ii) erlotinib on day 1 at a dose of 150 mg/day orally.

76. The method of any one of claims 1-46, wherein the human subject is administered a HER2 inhibitor.

77. The method of claim 76, wherein the HER2 inhibitor is trastuzumab.

78. The method of claim 77, wherein the human subject has been diagnosed with HER2 positive breast cancer.

79. The method of claim 77, wherein the human subject has been diagnosed with HER2 positive gastric cancer.

80. The method of claim 78 or 79, wherein the human subject had failed initial therapy for advanced or metastatic disease.

81. The method of any one of claims 77, 78, and 80, wherein the human subject is administered (i) the anti-HER3 antibody or antigen-binding fragment by intravenous (IV) infusion on day 1 and repeated every 21 days; and (ii) trastuzumab at an initial loading dose of 4 mg/kg IV on day 2 over approximately 90 minutes followed by 2 mg/kg IV over approximately 30 minutes starting on day 8 and continuing weekly.

82. The method of any one of claims 77, 78, and 80, wherein the human subject is administered (i) the anti-HER3 antibody or antigen-binding fragment by intravenous (IV) infusion on day 1 and repeated every 21 days; and (ii) trastuzumab at a dose of 4 mg/kg or 2 mg/kg IV weekly.

83. The method of claim 82 wherein the anti-HER3 antibody or antigen-binding fragment and trastuzumab are administered approximately 2 hours apart on the same day.

84. The method of any one of claims 77, 78, and 80, wherein the human subject is administered (i) the anti-HER3 antibody or antigen-binding fragment by intravenous (IV) infusion on day 1 and repeated every 21 days; and (ii) trastuzumab at an initial loading dose of 8 mg/kg IV on day 2 over approximately 90 minutes followed by 6 mg/kg IV over approximately 30 minutes starting on day 8 and continuing every 3 weeks.

85. The method of any one of claims 77, 78, and 80, wherein the human subject is administered (i) the anti-HER3 antibody or antigen-binding fragment by intravenous (IV) infusion on day 1 and repeated every 21 days; and (ii) trastuzumab at a dose of 8 mg/kg or 6 mg/kg IV every 3 weeks.

86. The method of any one of claims 1-85, comprising administering to the human subject the anti-HER3 antibody or antigen-binding fragment thereof in a dose of 15 mg/kg.

87. The method of any one of claims 1-85, comprising administering to the human subject the anti-HER3 antibody or antigen-binding fragment thereof in a dose of 20 mg/kg.

88. The method of claim 86 or 87, wherein the anti-HER3 antibody or antigen-binding fragment comprises a VL comprising SEQ ID NO: 3 and a VH comprising SEQ ID NO: 2.

89. The method of claim 88, wherein the anti-HER3 antibody or antigen-binding fragment comprises a human IgG constant region.

90. The method of claim 89, wherein the human IgG constant region is a human IgG1 constant region.

91. The method of any one of claims 88, 89, and 90, wherein the anti-HER3 antibody or antigen-binding fragment comprises a human light chain constant region.

92. The method of claim 91, wherein the human light chain constant region is a human lambda constant region.

93. The method of any one of claims 89-92, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

94. The method of any one of claims 86-93, comprising administering to the human subject a anti-HER3 antibody.

95. The method of claim 94, wherein the anti-HER3 antibody is a monoclonal, fully human antibody.

96. A method of treating cancer in a human subject in need thereof, comprising administering to the human subject an amount of an anti-HER3 antibody or antigen-binding fragment thereof which specifically binds to an epitope within the extracellular domain of HER3, wherein the anti-HER3 antibody specifically binds to the same HER3 epitope as an antibody or antigen-binding fragment thereof comprising the heavy chain variable region (VH) and light chain variable region (VL) of CL16 or 2C2 or competitively inhibits HER3 binding by an antibody or antigen-binding fragment thereof comprising the VH and VL of CL16 or 2C2, and wherein the amount of the anti-HER3 antibody or antigen-binding fragment thereof is sufficient to achieve
(i) a Cₘᵢₙ of 50 μg/mL or greater antibody serum concentration; or
(ii) antibody serum concentration of 50 μg/mL or greater throughout a dosing interval.

97. The method of claim 96, wherein the VH and VL of CL16 comprise SEQ ID NOs: 2 and 1, respectively, and the VH and VL of 2C2 comprise SEQ ID NOs: 2 and 3, respectively.

98. The method of claim 96 or 97, wherein the anti-HER3 antibody or antigen-binding fragment thereof comprises an antibody VL, wherein the VL comprises the amino acid sequence:
[FW₁]X₁GSX₂SNIGLNYVS[FW₂]RNNQRPS[FW₃]AAWDDX₃X₄X₅GEX₆
[FW₄]
wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A).

99. The method of claim 98, wherein FW₁ comprises SEQ ID NO: 40 or 44, FW₂ comprises SEQ ID NO: 41, FW₃ comprises SEQ ID NO: 42, and FW₄ comprises SEQ ID NO: 43.

100. The method of claim 96, 97, or 98, wherein the anti-HER3 antibody or antigen-binding fragment thereof comprises an antibody VH, wherein the VH comprises the amino acid sequence:
[FW₅]YYYMQ[FW₆]X₇IGSSGGVTNYADSVKG[FW₇]VGLGDAFDI[FW₈]
wherein [FW₅], [FW₆], [FW₇] and [FW₈] represent VH framework regions, and wherein
X₇ represents amino acid residues Tyrosine (Y), Isoleucine (I) or Valine (V).

101. The method of claim 100, wherein FW₅ comprises SEQ ID NO: 36, FW₆ comprises SEQ ID NO: 37, FW₇ comprises SEQ ID NO: 38 and FW₈ comprises SEQ ID NO: 39.

102. The method of claim 96 or 97, wherein the anti-HER3 antibody or antigen-binding fragment comprises an antibody VL and an antibody VH, wherein the VL comprises the amino acid sequence:
[FW₁]X₁GSX₂SNIGLNYVS[FW₂]RNNQRPS[FW₃]AAWDDX₃X₄X₅GEX₆
[FW₄]
wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and
wherein the VH comprises the amino acid sequence:
[FW₅]YYYMQ[FW₆]X₇IGSSGGVTNYADSVKG[FW₇]VGLGDAFDI[FW₈]
wherein [FW₅], [FW₆], [FW_{D}] and [FW₈] represent VH framework regions, and wherein
X₇ represents amino acid residues Tyrosine (Y), Isoleucine (I) or Valine (V).

103. The method of claim 102, wherein FW₁ comprises SEQ ID NO: 40 or 44, FW₂ comprises SEQ ID NO: 41, FW₃ comprises SEQ ID NO: 42, FW₄ comprises SEQ ID NO: 43, FW₅ comprises SEQ ID NO: 36, FW₆ comprises SEQ ID NO: 37, FW₇ comprises SEQ ID NO: 38, and FW₈ comprises SEQ ID NO: 39.

104. The method of claim 102, wherein the anti-HER3 antibody or antigen-binding fragment thereof comprises an antibody VL, wherein the VL comprises (i) a VL complementarity determining region-1 (VL-CDR1) amino acid sequence identical to, or identical except for four, three, two or one amino acid substitutions to: SEQ ID NO: 18, SEQ ID NO: 19, or SEQ ID NO: 20; (ii) a VL complementarity determining region-2 (VL-CDR2) amino acid sequence identical to, or identical except for four, three, two or one amino acid substitutions to SEQ ID NO: 21; and (iii) a complementarity determining region-3 (VL-CDR3) amino acid sequence identical to, or identical except for four, three, two, or one amino acid substitutions to: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

105. The method of claim 102, wherein the anti-HER3 antibody or antigen-binding fragment thereof comprises an antibody VH, wherein the VH comprises (i) a complementarity determining region-1 (VH-CDR1) amino acid sequence identical to, or identical except for four, three, two, or one amino acid substitutions to SEQ ID NO: 31; (ii) a complementarity determining region-2 (VH-CDR2) amino acid sequence identical to, or identical except for four, three, two, or one amino acid substitutions to: SEQ ID NO: 32, SEQ ID NO: 33, or SEQ ID NO: 34; and (iii) a complementarity determining region-3 (VH-CDR3) amino acid sequence identical to, or identical except for four, three, two, or one amino acid substitutions to SEQ ID NO: 35.

106. The method of claim 96, wherein the anti-HER3 antibody or antigen-binding fragment thereof comprises an antibody VL, wherein the VL comprises VL-CDR1, VL-CDR2, and VL-CDR3 amino acid sequences identical to, or identical except for four, three, two, or one amino acid substitutions in one or more of the VL-CDRS to: SEQ ID NOs: 18, 21 and 22, SEQ ID NOs: 18, 21, and 26, SEQ ID NOs: 18, 21, and 27, SEQ ID NOs: 20, 21, and 22, SEQ ID NOs: 19, 21, and 22, SEQ ID NOs: 18, 21, and 25, SEQ ID NOs: 18, 21, and 28, SEQ ID NOs: 18, 21, and 29, SEQ ID NOs: 18, 21, and 30, SEQ ID NOs: 18, 21, and 23, SEQ ID NOs: 19, 21, and 23, SEQ ID NOs: 20, 21, and 23, SEQ ID NOs: 18, 21, and 24, or SEQ ID NOs: 18, 21, and 25, respectively.

107. The method of claim 96, wherein the anti-HER3 antibody or antigen-binding fragment thereof comprises an antibody VH, wherein the VH comprises VH-CDR1, VH-CDR2, and VH-CDR3 amino acid sequences identical to, or identical except for four, three, two, or one amino acid substitutions in one or more of the VH-CDRS to: SEQ ID NOs: 31, 32 and 35, SEQ ID NOs: 31, 33, and 35, or SEQ ID NOs: 31, 34, and 35, respectively.

108. The method of claim 96, wherein the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

109. The method of claim 96, wherein the anti-HER3 antibody or antigen-binding fragment comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

110. The method of claim 96, wherein the anti-HER3 antibody or antigen-binding fragment comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

111. The method of claim 96, wherein the anti-HER3 antibody or antigen-binding fragment comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

112. The method of claim 96, wherein the anti-HER3 antibody or antigen-binding fragment thereof comprises a VL comprising the VL consensus sequence provided in Table 3 and a VH comprising the VH consensus sequence provided in Table 3.

113. The method of claim 96, wherein the anti-HER3 antibody or antigen-binding fragment comprises a VL comprising SEQ ID NO: 3 and a VH comprising SEQ ID NO: 2.

114. The method of any one of claims 96-113, wherein the anti-HER3 antibody or antigen-binding fragment comprises a heavy chain constant region or fragment thereof.

115. The method of claim 114, wherein the heavy chain constant region or fragment thereof is an IgG constant region.

116. The method of claim 115, wherein the IgG constant region is selected from an IgG1 constant region, an IgG2 constant region, an IgG3 constant region and an IgG4 constant region.

117. The method of claim 115, wherein the IgG constant region is a human IgG1 constant region.

118. The method of any one of claims 95-116, wherein the anti-HER3 antibody or antigen-binding fragment comprises a light chain constant region selected from the group consisting of a human kappa constant region and a human lambda constant region.

119. The method of claim 118, wherein the light chain constant region is a human lambda constant region.

120. The method of any one of claims 115-119, wherein the IgG constant domain comprises one or more amino acid substitutions relative to a wild-type IgG constant domain wherein the modified IgG has an increased half-life compared to the half-life of an IgG having the wild-type IgG constant domain.

121. The method of claim 120, wherein the IgG constant domain comprises one or more amino acid substitutions of amino acid residues at positions 251-257, 285-290, 308-314, 385-389, and 428-436, wherein the numbering is according to the EU index as set forth in Kabat.

122. The method of claim 121, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions,
wherein the numbering is according to the EU index as set forth in Kabat.

123. The method of claim 122, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

124. The method of any one of claim 121, 122, or 123, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

125. The method of claim 124, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

126. The method of claim 124, wherein the amino acid at position 257 is substituted with Leucine (L), and the amino acid at Kabat position 434 is substituted with Tyrosine (Y), and wherein the numbering is according to the EU index as set forth in Kabat.

127. The method of claim 125, wherein the amino acid at Kabat position 428 is substituted with Leucine (L), and the amino acid at Kabat position 434 is substituted with Serine (S).

128. A method of treating cancer in a human subject in need thereof, comprising administering to the subject an amount of an anti-HER3 antibody or antigen-binding fragment thereof which specifically binds to an epitope within the extracellular domain of HER3, wherein the anti-HER3 antibody or antigen binding fragment comprises an antibody VL of SEQ ID NO:3, an antibody VH of SEQ ID NO: 2, and an IgG1 constant region of SEQ ID 46, and wherein the amount of the anti-HER3 antibody or antigen-binding fragment thereof is sufficient to achieve
(i) a Cₘᵢₙ of 50 μg/mL or greater antibody serum concentration; or
(ii) antibody serum concentration of 50 μg/mL or greater throughout a dosing interval.

129. The method of claim 128 wherein the human IgG constant region comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

130. The method of any one of claims 96-129, wherein the antibody is a fully human antibody, a humanized antibody, a chimeric antibody, a monoclonal antibody, a polyclonal antibody, a recombinant antibody, a multispecific antibody, or an antigen-binding fragment thereof.

131. The method of claim 130, wherein the anti-HER3 antibody is a monoclonal, fully human antibody.

132. The method of any one of claims 96-131, which antigen-binding fragment is Fv, Fab, F(ab′)2, Fab′, dsFv, scFv, and sc(Fv)2.

133. The method of any one of claims 96-132, which anti-HER3 antibody or antigen-binding fragment is conjugated to at least one heterologous agent.

134. The method of any one of claims 1-133, wherein the amount of the anti-HER3 antibody or antigen-binding fragment thereof administered is sufficient to achieve:
(i) a Cₘᵢₙ of 50 μg/mL or greater antibody serum concentration; or
(ii) an antibody serum concentration of 50 μg/mL or greater throughout a dosing interval of 1 week, 2 weeks, 3 weeks or 4 weeks.

135. The method of any one of claims 1 and 96-134, wherein the amount of the anti-HER3 antibody or antigen-binding fragment thereof administered is 5 mg/kg, 10 mg/kg, 15 mg/kg, or 20 mg/kg every week, every 2 weeks, every 3 weeks or every 4 weeks.

136. The method of claim 135, wherein the amount of the anti-HER3 antibody or antigen-binding fragment thereof administered is 5 mg/kg every 3 weeks.

137. The method of claim 135, wherein the amount of the anti-HER3 antibody or antigen-binding fragment thereof administered is 10 mg/kg every 3 weeks.

138. The method of claim 135, wherein the amount of the anti-HER3 antibody or antigen-binding fragment thereof administered is 15 mg/kg every 3 weeks.

139. The method of claim 135, wherein the amount of the anti-HER3 antibody or antigen-binding fragment thereof administered is 20 mg/kg every 3 weeks.

140. The method of claim 135, wherein the amount of the anti-HER3 antibody or antigen-binding fragment thereof administered is 5 mg/kg every 4 weeks.

141. The method of claim 135, wherein the amount of the anti-HER3 antibody or antigen-binding fragment thereof administered is 10 mg/kg every 4 weeks.

142. The method of claim 135, wherein the amount of the anti-HER3 antibody or antigen-binding fragment thereof administered is 15 mg/kg every 4 weeks.

143. The method of claim 135, wherein the amount of the anti-HER3 antibody or antigen-binding fragment thereof administered is 20 mg/kg every 4 weeks.

144. The method of claim 135, wherein the amount of the anti-HER3 antibody or antigen-binding fragment thereof administered is 5 mg/kg every 2 weeks.

145. The method of claim 135, wherein the amount of the anti-HER3 antibody or antigen-binding fragment thereof administered is 10 mg/kg every 2 weeks.

146. The method of claim 135, wherein the amount of the anti-HER3 antibody or antigen-binding fragment thereof administered is 15 mg/kg every 2 weeks.

147. The method of claim 135, wherein the amount of the anti-HER3 antibody or antigen-binding fragment thereof administered is 20 mg/kg every 2 weeks.

148. The method of claim 135, wherein the amount of the anti-HER3 antibody or antigen-binding fragment thereof administered is 5 mg/kg every week.

149. The method of claim 135, wherein the amount of the anti-HER3 antibody or antigen-binding fragment thereof administered is 10 mg/kg every week.

150. The method of claim 135, wherein the amount of the anti-HER3 antibody or antigen-binding fragment thereof administered is 15 mg/kg every week.

151. The method of claim 135, wherein the amount of the anti-HER3 antibody or antigen-binding fragment thereof administered is 20 mg/kg every week.

152. The method of any one of claims 1 and 96-134, wherein the amount of the anti-HER3 antibody or antigen-binding fragment thereof administered is (i) 1200 mg every week, every 2 weeks, every 3 weeks, or every 4 weeks; (ii) 5 mg/kg weekly; (iii) 500 mg weekly; (iv) 10 mg/kg every 2 weeks or every 3 weeks; or (v) 1000 mg every 2 weeks or every 3 weeks.

153. The method of claim 152, wherein the amount of the anti-HER3 antibody or antigen-binding fragment thereof administered is 1200 mg every 3 weeks.

154. The method of claim 152, wherein the amount of the anti-HER3 antibody or antigen-binding fragment thereof administered is 1200 mg every 4 weeks.

155. The method of claim 152, wherein the amount of the anti-HER3 antibody or antigen-binding fragment thereof administered is 1200 mg every 2 weeks.

156. The method of claim 152, wherein the amount of the anti-HER3 antibody or antigen-binding fragment thereof administered is 1200 mg every week.

157. The method of claim 152, wherein the amount of the anti-HER3 antibody or antigen-binding fragment thereof administered is 500 mg every week.

158. The method of claim 152, wherein the amount of the anti-HER3 antibody or antigen-binding fragment thereof administered is 1000 mg every 2 weeks.

159. The method of claim 152, wherein the amount of the anti-HER3 antibody or antigen-binding fragment thereof administered is 1000 mg every 3 weeks.

160. The method of any one of claims 96-134, wherein the amount of the anti-HER3 antibody or antigen-binding fragment thereof administered is sufficient to achieve an antibody serum concentration in the range of 50 μg/mL to 500 μg/mL.

161. The method of claim 152, wherein the amount of the anti-HER3 antibody or antigen-binding fragment thereof administered is sufficient to achieve an antibody serum concentration in the range of 50 μg/mL to 250 μg/mL.

162. The method of any one of claims 1, 96-134, 160, and 161, wherein the human subject is administered a loading dose of the anti-HER3 antibody or antigen-binding fragment thereof that is higher than a subsequent dose of the anti-HER3 antibody or antigen-binding fragment thereof.

163. The method of claim 162, wherein the loading dose is administered 1 week, 2 weeks, 3 weeks, or 4 weeks prior to the subsequent dose of the anti-HER3 antibody or antigen-binding fragment thereof.

164. The method of claim 162 or 163, wherein the subsequent dose of the anti-HER3 antibody or antigen-binding fragment thereof is 5 mg/kg, 10 mg/kg, 15 mg/kg, or 20 mg/kg every week, every 2 weeks, every 3 weeks or every 4 weeks.

165. The method of claim 164, wherein the subsequent dose of the anti-HER3 antibody or antigen-binding fragment thereof administered is 5 mg/kg every 3 weeks.

166. The method of claim 164, wherein the subsequent dose of the anti-HER3 antibody or antigen-binding fragment thereof administered is 10 mg/kg every 3 weeks.

167. The method of claim 164, wherein the subsequent dose of the anti-HER3 antibody or antigen-binding fragment thereof administered is 15 mg/kg every 3 weeks.

168. The method of claim 164, wherein the subsequent dose of the anti-HER3 antibody or antigen-binding fragment thereof administered is 20 mg/kg every 3 weeks.

169. The method of claim 164, wherein the subsequent dose of the anti-HER3 antibody or antigen-binding fragment thereof administered is 5 mg/kg every 4 weeks.

170. The method of claim 164, wherein the subsequent dose of the anti-HER3 antibody or antigen-binding fragment thereof administered is 10 mg/kg every 4 weeks.

171. The method of claim 164, wherein the subsequent dose of the anti-HER3 antibody or antigen-binding fragment thereof administered is 15 mg/kg every 4 weeks.

172. The method of claim 164, wherein the subsequent dose of the anti-HER3 antibody or antigen-binding fragment thereof administered is 20 mg/kg every 4 weeks.

173. The method of claim 164, wherein the subsequent dose of the anti-HER3 antibody or antigen-binding fragment thereof administered is 5 mg/kg every 2 weeks.

174. The method of claim 164, wherein the subsequent dose of the anti-HER3 antibody or antigen-binding fragment thereof administered is 10 mg/kg every 2 weeks.

175. The method of claim 164, wherein the subsequent dose of the anti-HER3 antibody or antigen-binding fragment thereof administered is 15 mg/kg every 2 weeks.

176. The method of claim 164, wherein the subsequent dose of the anti-HER3 antibody or antigen-binding fragment thereof administered is 20 mg/kg every 2 weeks.

177. The method of claim 164, wherein the subsequent dose of the anti-HER3 antibody or antigen-binding fragment thereof administered is 5 mg/kg every week.

178. The method of claim 164, wherein the subsequent dose of the anti-HER3 antibody or antigen-binding fragment thereof administered is 10 mg/kg every week.

179. The method of claim 164, wherein the subsequent dose of the anti-HER3 antibody or antigen-binding fragment thereof administered is 15 mg/kg every week.

180. The method of claim 164, wherein the subsequent dose of the anti-HER3 antibody or antigen-binding fragment thereof administered is 20 mg/kg every week.

181. The method of any one of claims 1 and 96-134, wherein the anti-HER3 antibody or antigen-binding fragment thereof is administered intravenously.

182. The method of claim 181, wherein the anti-HER3 antibody or antigen-binding fragment thereof is administered as a 60-minute intravenous infusion.

183. The method of any one of claims 96-182, further comprising administering to the subject a therapeutic effective amount of a MEK inhibitor.

184. The method of claim 183, wherein the MEK inhibitor is trametinib.

185. The method of any one of claims 96-184, wherein the cancer is selected from the group consisting of melanoma, thyroid cancer, colon cancer, non small cell lung cancer, pancreatic cancer, gastric cancer, breast cancer, and head and neck cancer.

186. The method of claim 185, wherein the cancer comprises cells comprising a KRAS mutation.

187. The method of claim 186, wherein the KRAS mutation comprises a mutation of codon 12 of a human KRAS gene.

188. The method of any one of claims 96-187, further comprising administering to the human subject a therapeutic effective amount of a BRAF inhibitor.

189. The method of claim 188, wherein the BRAF inhibitor is vemurafenib.

190. The method of claim 188 or 189, wherein the cancer comprises cells comprising a BRAF mutation.

191. The method of claim 190, wherein the BRAF mutation is the BRAF V600E mutation or the BRAF V600K mutation.

192. The method of any one of claims 188-191, wherein the BRAF inhibitor is an antibody or antigen binding fragment thereof.

193. The method of claim any one of claims 188-192, wherein the human subject has been diagnosed with melanoma or BRAF mutated melanoma.

194. The method of claim 193, wherein the human subject is administered (i) the anti-HER3 antibody or antigen-binding fragment by intravenous (IV) infusion on day 1 and repeated every 21 days; and (ii) vemurafenib at a dose of 960 mg approximately every 12 hours starting on day 2.

195. The method of any one of claims 96-182, further comprising administering to the human subject a therapeutically effective amount of an EGFR inhibitor.

196. The method of claim 195, wherein the EGFR inhibitor is cetuximab.

197. The method of claim 195 or 196, wherein the human subject has been diagnosed with SCCHN.

198. The method of claim 197, wherein the SCCHN is HPV positive.

199. The method of claim 197, wherein the SCCHN is HPV negative.

200. The method of claim 195 or 196, wherein the human subject has been diagnosed with colon cancer.

201. The method of any one of claims 195 and 197-200, wherein EGFR inhibitor is AZD9291.

202. The method of any one of claims 195-200, wherein the human subject is administered (i) the anti-HER3 antibody or antigen-binding fragment by intravenous (IV) infusion on day 1 and repeated every 21 days; and (ii) cetuximab at an initial loading dose of 400 mg/m² on day 2 over approximately 2 hours followed by weekly doses of 250 mg/m² over approximately 60 minutes starting on day 8 and continuing weekly.

203. The method of any one of claims 195-201, wherein the human subject is administered (i) the anti-HER3 antibody or antigen-binding fragment by intravenous (IV) infusion on day 1 and repeated every 21 days; and (ii) cetuximab on day 1 at a dose of 400 mg/m² or 250 mg/m² weekly or at an initial loading dose of 400 mg/m² followed by a weekly dose of 250 mg/m².

204. The method of claim 203, wherein the anti-HER3 antibody or antigen-binding fragment and the cetuximab are administered approximately 2 hours apart on the same day.

205. The method of claim 195, wherein the EGFR inhibitor is erlotinib.

206. The method of claim 195 or 205, wherein the human subject has been diagnosed with non-small cell lung cancer.

207. The method of claim 205 or 206, wherein the human subject is administered (i) the anti-HER3 antibody or antigen-binding fragment by intravenous (IV) infusion on day 1 and repeated every 21 days; and (ii) erlotinib on day 2 at a dose of 150 mg/day orally or 10 mg/day orally.

208. The method of claim 205 or 206, wherein the human subject is administered (i) the anti-HER3 antibody or antigen-binding fragment by intravenous (IV) infusion on day 1 and repeated every 21 days; and (ii) erlotinib on day 1 at a dose of 150 mg/day orally or 100 mg/day orally.

209. The method of any one of claims 96-182, further comprising administering to the human subject a therapeutically effective amount of a HER2 inhibitor.

210. The method of claim 209, wherein the HER2 inhibitor is trastuzumab.

211. The method of claim 209 or 210, wherein the human subject has been diagnosed with HER2 positive breast cancer.

212. The method of claim 209 or 210, wherein the human subject has been diagnosed with HER2 positive gastric cancer.

213. The method of any one of claims 96-212, wherein the human subject had failed initial therapy for advanced or metastatic disease.

214. The method of any one of claims 210-213, wherein the human subject is administered (i) the anti-HER3 antibody or antigen-binding fragment by intravenous (IV) infusion on day 1 and repeated every 21 days; and (ii) trastuzumab at an initial loading dose of 4 mg/kg IV on day 2 over approximately 90 minutes followed by 2 mg/kg IV over approximately 30 minutes starting on day 8 and continuing weekly.

215. The method of any one of claims 210-213, wherein the human subject is administered (i) the anti-HER3 antibody or antigen-binding fragment by intravenous (IV) infusion on day 1 and repeated every 21 days; and (ii) trastuzumab at a dose of 4 mg/kg or 2 mg/kg IV weekly.

216. The method of claim 215 wherein the anti-HER3 antibody or antigen-binding fragment and trastuzumab are administered approximately 2 hours apart on the same day.

217. The method of any one of claims 210-213, wherein the human subject is administered (i) the anti-HER3 antibody or antigen-binding fragment by intravenous (IV) infusion on day 1 and repeated every 21 days; and (ii) trastuzumab at an initial loading dose of 8 mg/kg IV on day 2 over approximately 90 minutes followed by 6 mg/kg IV over approximately 30 minutes starting on day 8 and continuing every 3 weeks.

218. The method of any one of claims 210-213, wherein the human subject is administered (i) the anti-HER3 antibody or antigen-binding fragment by intravenous (IV) infusion on day 1 and repeated every 21 days; and (ii) trastuzumab at a dose of 8 mg/kg or 6 mg/kg IV every 3 weeks.

219. The method of any one of claims 96-218, further comprising measuring the concentration of a pharmacodynamic marker in a sample from the human subject, wherein the pharmacodynamic marker is soluble HER3, TWEAK, TWEAK receptor, Neurexophilin-1, IL-18 binding protein, ANGL4, IL-18 receptor 1, or Kallikrein 7.

220. The method of claim 219, wherein the pharmacodynamic marker is measured prior to and after administration of the anti-HER3 antibody or antigen-binding fragment.

221. The method of claim 219 or 220, wherein circulating concentration of the pharmacodynamic marker is measured.

222. The method of any one of claims 96-221, comprising administering to the human subject an amount of the anti-HER3 antibody or antigen-binding fragment thereof is sufficient to achieve a Cₘᵢₙ of 50 μg/mL or greater antibody serum concentration.

223. The method of any one of claims 96-221, comprising administering to the human subject an amount of the anti-HER3 antibody or antigen-binding fragment thereof is sufficient to achieve an antibody serum concentration of 50 μg/mL or greater throughout a dosing interval.

224. The method of claim 222 or 223, wherein the anti-HER3 antibody or antigen-binding fragment comprises a VL comprising SEQ ID NO: 3 and a VH comprising SEQ ID NO: 2.

225. The method of claim 224, wherein the anti-HER3 antibody or antigen-binding fragment comprises a human IgG constant region.

226. The method of claim 225, wherein the human IgG constant region is a human IgG1 constant region.

227. The method of any one of claim 224, 225, or 226, wherein the anti-HER3 antibody or antigen-binding fragment comprises a human light chain constant region.

228. The method of claim 227, wherein the human light chain constant region is a human lambda constant region.

229. The method of any one of claims 225-228, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

230. The method of any one of claims 222-229, comprising administering to the human subject an anti-HER3 antibody.

231. The method of claim 230, wherein the anti-HER3 antibody is a monoclonal, fully human antibody.

232. A method of monitoring treatment with an anti-HER3 antibody or antigen-binding fragment thereof which specifically binds to an epitope within the extracellular domain of HER3, of a human subject diagnosed with cancer, comprising measuring concentration of a pharmacodynamic marker in a sample from the human subject, wherein the pharmacodynamic marker is soluble HER3, TWEAK, TWEAK receptor, Neurexophilin-1, IL-18 binding protein, ANGL4, IL-18 receptor 1, or Kallikrein 7.

233. The method of claim 232, wherein the anti-HER3 antibody or antigen-binding fragment comprises a VL comprising SEQ ID NO: 3 and a VH comprising SEQ ID NO: 2.

234. The method of claim 232 or 233, wherein the pharmacodynamic marker is measured prior to, and after, administration of the anti-HER3 antibody or antigen-binding fragment.

235. The method of claim 232, 233, or 234, wherein circulating concentration of the pharmacodynamic marker is measured.

236. The method of any one of claims 232-235, wherein an increase or decrease in soluble HER3 circulating concentration following treatment with the anti-HER3 antibody or antigen-binding fragment thereof is indicative of treatment.

237. The method of any one of claims 231-234, wherein an increase in circulating concentration of TWEAK or TWEAK receptor following treatment with the anti-HER3 antibody or antigen-binding fragment thereof is indicative of treatment.

238. The method of any one of claims 231-234, wherein a decrease in circulating concentration of Neurexophilin-1, IL-18 binding protein, IL-18 receptor 1, ANGL4, or Kallinkrein 7 following treatment with the anti-HER3 antibody or antigen-binding fragment thereof is indicative of treatment.

239. A method of treating thyroid cancer, comprising administering to a subject in need thereof (a) a BRAF inhibitor, and (b) an anti-HER3 antibody or antigen-binding fragment thereof, wherein the BRAF inhibitor and anti-HER3 antibody increase the uptake of radioiodine by said subject.

240. The method of claim 239, wherein the method further comprises administering a therapeutically effective dose of radioiodine.

241. The method of claim 239 or 240, wherein the thyroid cancer is a BRAF mutated thyroid cancer.

242. The method of claim 241, wherein the BRAF mutated thyroid cancer is a V600 BRAF mutated cancer.

243. The method of claim 242, wherein the BRAF mutated thyroid cancer is a V600E BRAF mutated cancer.

244. The method of claim 239 or 240, wherein the thyroid cancer is a radioiodine refractory thyroid cancer.

245. The method of claim 244, wherein the radioiodine refractory thyroid cancer is a BRAF mutated radioiodine refractory thyroid cancer.

246. The method of claim 245, wherein the BRAF mutated radioiodine refractory thyroid cancer is a V600 BRAF mutated cancer.

247. The method of claim 246, wherein the BRAF mutated radioiodine refractory thyroid cancer is a V600E BRAF mutated cancer.

248. The method of any one of claim 239-247, wherein the thyroid cancer is a differentiated thyroid carcinoma of follicular origin.

249. The method of any one of claim 239-247, wherein the thyroid cancer is a differentiated thyroid carcinoma of papillary origin.

250. The method of claim 249, wherein the differentiated carcinoma of papillary origin is a Hurthle cell carcinoma.

251. The method of any one of claims 239-250, wherein the BRAF inhibitor is vemurafenib.

252. The method of any one of claims 239-251, wherein the radioiodine is ¹³¹I.

253. The method of any one of claims 239-252, wherein the anti-HER3 antibody or antigen-binding fragment thereof specifically binds to the same HER3 epitope as an antibody or antigen-binding fragment thereof comprising the heavy chain variable region (VH) and light chain variable region (VL) of CL16 or 2C2.

254. The method of claim 253, wherein the VH and VL of CL16 comprise SEQ ID NOs: 2 and 1, respectively, and the VH and VL of 2C2 comprise SEQ ID NOs: 2 and 3, respectively.

255. The method of any one of claims 239-254, wherein the anti-HER3 antibody or antigen-binding fragment thereof is affinity matured.

256. The method of any one of claims 239-255, wherein the anti-HER3 antibody or antigen-binding fragment thereof comprises an antibody VL and an antibody VH, wherein the VL comprises the amino acid sequence:
[FW₁]X₁GSX₂SNIGLNYVS[FW₂]RNNQRPS[FW₃]AAWDDX₃X₄X₅GEX₆
[FW₄]
wherein [FW₁], [FW₂], [FW₃] and [FW₄] represent VL framework regions, and wherein
(a) X₁ represents amino acid residues Arginine (R) or Serine (S),
(b) X₂ represents amino acid residues Serine (S) or Leucine (L),
(c) X₃ represents amino acid residues Serine (S) or Glycine (G),
(d) X₄ represents amino acid residues Leucine (L) or Proline (P),
(e) X₅ represents amino acid residues Arginine (R), Isoleucine (I), Proline (P) or Serine (S), and
(f) X₆ represents amino acid residues Valine (V) or Alanine (A), and
wherein the VH comprises the amino acid sequence:
[FW₅]YYYMQ[FW₆]X₇IGSSGGVTNYADSVKG[FW₇]VGLGDAFDI[FW₈]
wherein [FW₅], [FW₆], [FW_{D}] and [FW₈] represent VH framework regions, and wherein
X₇ represents amino acid residues Tyrosine (Y), Isoleucine (I) or Valine (V).

257. The method of claim 256, wherein FW₁ comprises SEQ ID NO: 40 or 44, FW₂ comprises SEQ ID NO: 41, FW₃ comprises SEQ ID NO: 42, FW₄ comprises SEQ ID NO: 43, FW₅ comprises SEQ ID NO: 36, FW₆ comprises SEQ ID NO: 37, FW₇ comprises SEQ ID NO: 38, and FW₈ comprises SEQ ID NO: 39.

258. The method of any one of claims 239-252, wherein the anti-HER3 antibody or antigen-binding fragment thereof comprises a VL and a VH comprising VL-CDR1, VL-CRD2, VL-CDR3, VH-CDR1, VH-CDR2, and VH-CDR3 amino acid sequences identical or identical except for four, three, two, or one amino acid substitutions in one or more CDRs to: SEQ ID NOs: 18, 21, 22, 31, 32, and 35, SEQ ID NOs: 18, 21, 26, 31, 32 and 35, SEQ ID NOs: 18, 21, 27, 31, 32 and 35, SEQ ID NOs: 20, 21, 22, 31, 32 and 35, SEQ ID NOs: 19, 21, 22, 31, 32 and 35, SEQ ID NOs: 18, 21, 25, 31, 32 and 35, SEQ ID NOs: 18, 21, 28, 31, 32 and 35, SEQ ID NOs: 18, 21, 29, 31, 32 and 35, SEQ ID NOs: 18, 21, 30, 31, 32 and 35, SEQ ID NOs: 18, 21, 23, 31, 32 and 35, SEQ ID NOs: 19, 21, 23, 31, 32 and 35, SEQ ID NOs: 20, 21, 23, 31, 32 and 35, SEQ ID NOs: 18, 21, 24, 31, 32 and 35, or SEQ ID NOs: 18, 21, 25, 31, 32 and 35, respectively.

259. The method of any one of claims 239-252, wherein the anti-HER3 antibody or antigen-binding fragment thereof comprises an antibody VL and an antibody VH, wherein the VL comprises an amino acid sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17.

260. The method of any one of claims 239-252, wherein the anti-HER3 antibody or antigen-binding fragment thereof comprises an antibody VL and an antibody VH, wherein the VH comprises an amino acid sequence at least about 90% to about 100% identical to a reference amino acid sequence selected from the group consisting of SEQ ID NO: 2, SEQ ID NO: 12 and SEQ ID NO: 13.

261. The method of any one of claims 239-252, wherein the anti-HER3 antibody or antigen-binding fragment thereof comprises a VL comprising a sequence at least about 90% to about 100% identical to a reference amino acid sequence selected from the group consisting of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, and SEQ ID NO: 17, and wherein the anti-HER3 antibody or antigen-binding fragment comprises a VH comprising a sequence at least about 90% to about 100% identical to a reference amino acid sequence selected from the group consisting of SEQ ID NO: 2, SEQ ID NO: 12 and SEQ ID NO: 13.

262. The method of any one of claims 239-252, wherein the anti-HER3 antibody or antigen-binding fragment thereof comprises a VL comprising the VL consensus sequence provided in Table 3 and a VH comprising the VH consensus sequence provided in Table 3.

263. The method of any one of claims 239-252, wherein the anti-HER3 antibody or antigen-binding fragment thereof comprises a VL comprising SEQ ID NO: 3 and a VH comprising SEQ ID NO: 2.

264. The method of any one of claims 239-252, wherein the anti-HER3 antibody or antigen-binding fragment thereof comprises a human heavy chain constant region or fragment thereof.

265. The method of claim 264, wherein the heavy chain constant region or fragment thereof is an IgG constant region.

266. The method of claim 265, wherein the IgG constant region is selected from an IgG1 constant region, an IgG2 constant region, an IgG3 constant region and an IgG4 constant region.

267. The method of claim 265, wherein the IgG constant region is an IgG1 constant region.

268. The method of any one of claims 256-266, wherein the anti-HER3 antibody or antigen-binding fragment comprises a light chain constant region selected from the group consisting of a human kappa constant region and a human lambda constant region.

269. The method of claim 268, wherein the anti-HER3 antibody or antigen-binding fragment comprises a human lambda constant region.

270. The method of any one of claims 264-269, wherein the IgG constant domain comprises one or more amino acid substitutions relative to a wild-type IgG constant domain wherein the modified IgG has an increased half-life compared to the half-life of an IgG having the wild-type IgG constant domain.

271. The method of claim 270, wherein the IgG constant domain comprises one or more amino acid substitutions of amino acid residues at positions 251-257, 285-290, 308-314, 385-389, and 428-436, wherein the numbering is according to the EU index as set forth in Kabat.

272. The method of claim 271, wherein at least one IgG constant domain amino acid substitution is selected from the group consisting of:
(a) substitution of the amino acid at position 252 with Tyrosine (Y), Phenylalanine (F), Tryptophan (W), or Threonine (T),
(b) substitution of the amino acid at position 254 with Threonine (T),
(c) substitution of the amino acid at position 256 with Serine (S), Arginine (R), Glutamine (Q), Glutamic acid (E), Aspartic acid (D), or Threonine (T),
(d) substitution of the amino acid at position 257 with Leucine (L),
(e) substitution of the amino acid at position 309 with Proline (P),
(f) substitution of the amino acid at position 311 with Serine (S),
(g) substitution of the amino acid at position 428 with Threonine (T), Leucine (L), Phenylalanine (F), or Serine (S),
(h) substitution of the amino acid at position 433 with Arginine (R), Serine (S), Isoleucine (I), Proline (P), or Glutamine (Q),
(i) substitution of the amino acid at position 434 with Tryptophan (W), Methionine (M), Serine (S), Histidine (H), Phenylalanine (F), or Tyrosine, and
(j) a combination of two or more of said substitutions,
wherein the numbering is according to the EU index as set forth in Kabat.

273. The method of claim 271, wherein the human IgG constant domain comprises amino acid substitutions relative to a wild-type human IgG constant domain at positions 252, 254, and 256, wherein
(a) the amino acid at position 252 is substituted with Tyrosine (Y),
(b) the amino acid at position 254 is substituted with Threonine (T), and
(c) the amino acid at position 256 is substituted with Glutamic acid (E),
wherein the numbering is according to the EU index as set forth in Kabat.

274. The method of any one of claim 271, 272, or 273, wherein the amino acid at position 434 is substituted with an amino acid selected from the group consisting of Tryptophan (W), Methionine (M), Tyrosine (Y), and Serine (S), and wherein the numbering is according to the EU index as set forth in Kabat.

275. The method of claim 274, wherein the amino acid at position 428 is substituted with an amino acid selected from the group consisting of Threonine (T), Leucine (L), Phenylalanine (F), and Serine (S), and wherein the numbering is according to the EU index as set forth in Kabat.

276. The method of claim 275, wherein the amino acid at position 257 is substituted with Leucine (L), and the amino acid at Kabat position 434 is substituted with Tyrosine (Y), and wherein the numbering is according to the EU index as set forth in Kabat.

277. The method of claim 275, wherein the amino acid at Kabat position 428 is substituted with Leucine (L), and the amino acid at Kabat position 434 is substituted with Serine (S).

278. The method of any one of claims 238-251, wherein the anti-HER3 antibody or antigen-binding fragment thereof comprises an antibody VL of SEQ ID NO:3, an antibody VH of SEQ ID NO: 2, and an IgG1 constant region of SEQ ID 46.

279. The method of claim 278, wherein the human IgG constant region, comprises amino acid substitutions relative to a wild-type human IgG constant domain at positions 252, 254, and 256, wherein the numbering is according to the EU index as set forth in Kabat, and wherein
(a) the amino acid at position 252 is substituted with Tyrosine (Y),
(b) the amino acid at position 254 is substituted with Threonine (T), and
(c) the amino acid at position 256 is substituted with Glutamic acid (E).

280. The method of any one of claims 239-279, wherein the antibody is a fully human antibody, a humanized antibody, a chimeric antibody, a monoclonal antibody, a polyclonal antibody, a recombinant antibody, a multispecific antibody, or an antigen-binding fragment thereof.

281. The method of claim 280, wherein the anti-HER3 antibody is a monoclonal, fully human antibody.

282. The method of any one of claims 239-280, which antigen-binding fragment is Fv, Fab, F(ab′)2, Fab′, dsFv, scFv, and sc(Fv)2.

283. The method of any one of claims 239-282, which anti-HER3 antibody or antigen-binding fragment is conjugated to at least one heterologous agent.

284. The method of any one of claims 1-283, wherein the anti-HER3 antibody or antigen-binding fragment thereof is a monoclonal, human anti-HER3 antibody or antigen-binding fragment thereof, comprising a heavy chain variable region (VH) comprising a VH complementarity determining region (CDR)-1 (VH CDR-1) amino acid sequence of SEQ ID NO:31, a VH CDR-2 amino sequence of SEQ ID NO:32, and a VH CDR-3 amino acid sequence of SEQ ID NO:35; a human heavy chain IgG1 constant region comprising amino acid substitutions relative to a wild-type human IgG constant region at heavy chain amino acid positions 252, 254, and 256, wherein the amino acid at position 252 is substituted with tyrosine (Y), the amino acid at position 254 is substituted with threonine (T), and the amino acid at position 256 is substituted with glutamic acid (E), wherein the numbering is according to the EU index as set forth in Kabat; a light chain variable region (VL) comprising a VL CDR-1 amino acid sequence of SEQ ID NO:19, a VL CDR-2 amino sequence of SEQ ID NO:21, and a VL CDR-3 amino acid sequence of SEQ ID NO:23; and a human lambda light chain constant region.

285. The method of claim 284, wherein the HER3 antibody or antigen-binding fragment thereof is a monoclonal, human anti-HER3 antibody or antigen-binding fragment thereof, wherein the VH comprises the amino acid sequence of SEQ ID NO:2, and the VL comprises the amino acid sequence of SEQ ID NO:3.

286. The method of any one of claims 1-285, wherein the cancer expresses a high level of a HER3 ligand.

287. The method of claim 286 wherein the HER3 ligand is neuregulin 1 (NRG1).

288. The method of any one of claims 1-287, wherein, prior to administering the anti-HER3 antibody, the cancer is resistant to a BRAF inhibitor or a MEK inhibitor.

289. The method of claim 288, wherein the BRAF inhibitor is vemurafenib.

290. The method of claim 288, wherein the BRAF inhibitor is dabrafenib.

291. The method of claim 288, wherein the MEK inhibitor is trametinib.

292. The method of claim 288, wherein, prior to administering the anti-HER3 antibody, the cancer is resistant to a BRAF inhibitor and a MEK inhibitor.

293. The method of claim 292, wherein the BRAF inhibitor is vemurafenib.

294. The method of claim 292, wherein the BRAF inhibitor is dabrafenib.

295. The method of claim 292, wherein the MEK inhibitor is trametinib.

296. The method of claim 292, wherein the BRAF inhibitor is vemurafenib and the MEK inhibitor is trametinib.
